(19) United States
(12) Patent Application Publication
(10) Pub. No.: US 2008/0305119 A1
(43) Pub. Date: Dec. 11, 2008

(54) Modified Bacteriophage Vectors and Uses Thereof

(75) Inventors: Stephen Dewhurst, Rochester, NY (US); Christine N. Zanghi, Rochester, NY (US); Julie Richards Gunzler, Portland, OR (US)

Correspondence Address: FISH & RICHARDSON P.C., P.O BOX 1022, Minneapolis, MN, 55440-1022, US

(73) Assignee: UNIVERSITY OF ROCHESTER, Rochester, NY (US)

(21) Appl. No.: 11/577,515

(22) PCT Filed: Oct. 5, 2005
(86) PCT No.: PCT/US05/35428

§ 371 Date: May 22, 2007

Related U.S. Application Data
(60) Provisional application No. 60/623,743, filed on Oct. 29, 2004.

(51) Int. Cl. A61K 39/395; A61K 39/00; C12N 15/11; C12N 15/00; C12N 7/00; C12P 21/04; A61P 31/00

(52) U.S. Cl. 424/178.1; 424/199.1; 536/23.1; 435/320.1; 435/235.1; 435/69.1

(57) ABSTRACT

Provided herein are modified phage surface polypeptides, phages with modified surface polypeptides, nucleic acids that encode the modified surface polypeptides, and related vectors and phages comprising the vectors. The provided phage surface polypeptides optionally comprise one or more modifications, including, for example, one or more modifications to enhance targeting to an antigen-presenting cell and one or more modifications that destabilize a viral capsid. Further provided herein are methods of making a lambda phage with a modified surface polypeptide and methods of making a lambda phage with a plurality of modified surface polypeptides. Also provided herein are antigen delivery systems comprising the modified phages of the invention and methods of promoting an antigenic response in a subject by administering to the subject the antigen delivery system of the invention, alone or in combination with other immunization modalities.

### CROSS-REFERENCE TO RELATED APPLICATIONS

This application claims benefit of U.S. Provisional Application No. 60/623,743, filed Oct. 29, 2004, which is hereby incorporated herein by reference in its entirety.

### ACKNOWLEDGEMENTS

This invention was made with government support under Grants DAMD 17-01-1-0384 and F31AI054330 awarded by the Department of Defense (DOD)/Army and the National Institutes of Health (NIH). The government has certain rights in the invention.

### FIELD OF THE INVENTION

This invention relates generally to compositions and methods for the targeted delivery of antigens to antigen presenting cells and delivery of proteins of interest to target cells using modified lambda phage.

### BACKGROUND OF THE INVENTION

Bacteriophage lambda (λ) has certain appealing characteristics as an antigen delivery vector. Lambda is a dsDNA, temperate phage, 50 nm wide and about 150 nm long; this is a size comparable to most mammalian viruses, including HIV. Lambda can accept inserts and genomic deletions anywhere between 78% and 105% of the wild-type genome, allowing for insertion of up to 15 kb (Chauthaiwale, V. M., et al. 1992. Microbiol Rev 56:577-91). Finally, lambda is extremely stable under multiple storage conditions, including desiccation, and large-scale production of lambda is rapid and relatively inexpensive making it a versatile option for vaccine administration to low income nations (Jepson, C. D. and J. B. March. 2004. Vaccine 22:2413-9).

Lambda has been used in multiple peptide display experiments (Gupta, A., et al. 2003. Advances in Virus Research 60:421-67; Hoess, R. H. 2002. Curr Pharm Biotechnol 3:23-8; Mikawa, Y. G., et al. 1996. J Mol Biol 262:21-30; Santi, E., et al. 2000. J Mol Biol 296:497-508; Sternberg, N., and R. H. Hoess. 1995. Proc Natl Acad Sci USA 92:1609-13). Lambda has two identified platforms for peptide display. The tail protein, gpV, consists of 32 subunits important for infection of the bacterial host. After the deletion of a nonessential region of the carboxy terminus, protein and peptide fusions can be successfully displayed on gpV in low copy numbers (Hoess, R. H. 2002. Curr Pharm Biotechnol 3:23-8). The second lambda platform for peptide display is the coat protein gpD. The gpD capsid protein is 11.4 kDa and serves to stabilize the phage head after genomic insertion; there are between 405 and 420 copies of gpD per phage, allowing for higher copy numbers of the displayed peptide. Fusions of both the amino and carboxy terminus have been successfully displayed on the coat surface (Mikawa, Y. G., et al. 1996. J Mol Biol 262:21-30) but resolution of the crystal structure for gpD indicated that the carboxy terminus is well suited to peptide display (Yang, F., et al. 2000. Nat Struct Biol 7:230-7).

Sternberg and Hoess described a novel method for peptide display in lambda phage. Their base phage lacked gpD, but was stable due to a genomic size of 78.5% of wild type. In addition, the phage possessed a temperature sensitive mutation in the gene responsible for repressing the lytic lifecycle. Therefore, the phage could be stably grown as an E. coli lysogen and the lytic phage released when desired. Transformation of the lambda lysogens with a gpD-peptide expression plasmid resulted in gpD complementation of the phage in trans after lytic induction (Sternberg, N., and R. H. Hoess. 1995. Proc Natl Acad Sci USA 92:1609-13). Eguchi et al. adapted this system for gene delivery to mammalian cells (Eguchi, A., et al. 2001. J Biol Chem 276:26204-10). The Eguchi expression plasmids contained the protein transduction domain of HIV-1 Tat fused to the amino terminus of gpD. In addition, luciferase (luc) or green fluorescent protein (GFP) expression cassettes were inserted into a unique EcoRI restriction site of the lambda genome (λ D1180) to form λ D1180(gfp) and λ D1180(luc). Transformation and lytic induction of E. coli lysogens resulted in the formation of recombinant lambda displaying Tat-gpD peptide fusions and capable of delivering either GFP or luc for mammalian cell expression. Successful transduction and subsequent gene expression of COS-1 cells was demonstrated by luciferase assay and fluorescence microscopy. Site-specific GFP expression was also observed after injection of mice with 8.5×109 plaque forming units (pfu) of Tat phage (Eguchi, A., et al. 2001. J Biol Chem 276:26204-10).

Phage are inexpensive to produce and purify, genetically tractable, and have a substantial track record of safe use in humans and research animals in large quantities for the treatment of bacterial infections (Barrow, P., et al. 1998. Clin Diagn Lab Immunol 5:294-8; Barrow, P. A., and J. S. Soothill. 1997. Trends Microbiol 5:268-71; Dubos, R., et al. 1943. J Exp Med 78:161-168; Schoolnik, G. K., et al. 2004. Nature Biotechnology 22:505-6). The use of phage in vaccine delivery has been proposed, but the development of phage-based vaccines has centered on phage display of antigenic peptides linked to filamentous (M13) coat proteins. These vaccines have successfully induced antibody and some cytolytic responses in laboratory animals (Chen, X., et al. 2001. Nat Med 7:1225-31, De Berardinis, P., et al. 1999. Vaccine 17:1434-41; De Berardinis, P., et al. 2000. Nat Biotechnol 18:873-6), but the T-cell response is often weaker than those observed in mammalian viral vectors. Furthermore, these approaches are limited to short antigenic epitopes, due to the constraints on surface display of peptides on filamentous phage, and they do not permit new antigen synthesis in mammalian cells because the surface-modified phage lack a mammalian expression cassette. Immunization of mice and rabbits with non-targeted lambda carrying the gene for Hepatitis B surface antigen induced a potent anti-HBsAg response (March, J. B., et al. 2004. Vaccine 22:1666-71). These results indicate that even non-targeted phage can be internalized by antigen presenting cells.

Thus, although bacteriophage can express encoded genes within the transduced mammalian cell (Burg, M. A., et al 2002. Cancer Res 62:977-81, Di Giovine, M., et al. 2001. Virology 282:102-12, Eguchi, A., et al. 2001. J Biol Chem 276:26204-10, Larocca, D., and A. Baird. 2001. Drug Discov Today 6:793-801; Larocca, D., et al. 2002. Curr Pharm Biotechnol 3:45-57; Larocca, D., et al. 2002. Methods Mol Biol 185:393-401; Larocca, D., et al. 2001. Mol Ther 3:476-84; Larocca, D., et al. 1999. Faseb J 13:727-34; Larocca, D., et al. 1998. Hum Gene Ther 9:2393-9; Piersanti, S., et al. 2004. J Mol Med 82:467-76; Urbanelli, L., et al. 2001. J Mol Biol 313:965-76), needed within the art are modified bacteriophage that are targeted and that have a strong antigenic response.

Presented herein are modified recombinant lambda phage that will more efficiently transduce mammalian cells, and thereby elicit stronger antigenic responses to encoded antigens, for purposes of vaccination against infectious agents, cancer, neurologic diseases and other disorders.

### SUMMARY OF THE INVENTION

In accordance with the purpose(s) of this invention, as embodied and broadly described herein, this invention, in one aspect, relates to an antigen delivery system or gene delivery system comprising a modified phage and a phage encoded antigen or gene of interest. The invention relates to modified phage surface polypeptides, modified phages, nucleic acids that encode the modified surface polypeptides, and related vectors. The invention also relates to phage surface polypeptides with one or more modifications, including, for example, one or more modifications to enhance targeting to an antigen-presenting cell or other target cell and one or more modifications that destabilizes a viral capsid. Also provided herein are antigen delivery systems comprising the modified phages of the invention and methods of promoting an antigenic response in a subject by administering to the subject the antigen delivery system of the invention. Also provided are gene delivery systems comprising modified phages of the invention. Further provided herein are methods of making a lambda phage with a modified surface polypeptide, or a plurality of modified surface polypeptides. Also provided is a method of transducing a cell, comprising contacting the cell with a modified phage of the invention.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 shows E. coli expression plasmid for the production of gpD fusion proteins. The histidine tag was removed from pTrcHisA plasmid (Invitrogen, Carlsbad, Calif.) and an E. coli codon optimized gpD sequence was added after the trp/lac promoter. A 13 amino acid linker sequence joined gpD and the targeting peptide sequence. All targeting peptides were cloned between the unique KpnI and HindIII sites.

FIG. 2 shows expression of gpD fusion proteins. The indicated sequences 3JCLI4, Fnfn10, Chemerin peptide (Chem), scrambled chemerin peptide (ChemSc), C2 and ZZ domains were inserted at the carboxy terminus of gpD, as seen in FIG. 1. Cellular lysates were separated by SDS-PAGE and gpD fusions were identified by immunoblot with an anti-gpD antibody.

FIG. 3 shows trans-complementation of D-deficient lambda phage with modified gpD fusion proteins. The indicated gpD-encoding plasmids were transformed into λ D1180(luc) lysogens. The recombinant phage were purified by cesium chloride equilibrium gradient and titered on E. coli strain LE392. Phage (1×10⁸ pfu) were then lysed, separated by SDS/PAGE, and visualized on immunoblot with a polyclonal rabbit antiserum against gpD.

FIG. 4 shows that purified lambda phage bearing gpD-C2 and gpD-ZZ bind IgG. gpD phage incubated with anti-gpD rabbit antibody and anti-rabbit IgG-HRP antibody served as the positive control. Negative controls included gpD-ZZ and gpD-C2 phage with no antibody, gpD phage with just anti-rabbit IgG-HRP, and antibody without phage.

FIG. 5 shows enhanced transduction of DC-SIGN positive human cells using lambda phage bearing gpD-ZZ. Phage (1×10¹⁰ pfu) were pre-complexed with the indicated antibodies (2 μg/ml) for 1 hour at 37° C. The antibody-phage conjugates were then added to DC-SIGN expressing HeLa cells, for 2 hours at 37° C. and an MOI of 1×10⁵. All infections were performed in the presence of 100 μM chloroquine and included spinoculation-mediated enhancement of infection (centrifugation at 2000 rpm, at 30° C. for 40 minutes). Luciferase expression was measured at 48 hours post infection.

FIG. 6 shows that D-deficient lambda phage complemented with a wild-type gpD expression plasmid successfully mediates gene transfer in vivo. λD1180(luc) was propagated in E. coli cells that expressed wild-type gpD. The resulting phage (λ+gpD) was CsCl-gradient purified and injected intramuscularly (IM) or intradermally (ID) at 1×10¹¹ pfu or ID at 5×1010 pfu in 5-6 week old Balb/c mice. Controls were 1×10⁹ pfu of a luciferase-expressing adenovirus (positive) and 1×10¹⁰ pfu of wild type lambda (negative) injected IM. For imaging of luciferase expression, a luminescent substrate (Luciferin, 4.3 mg) was injected IP at 24 and 48 hours and mice were imaged using an IVIS imaging system (Xenogen, Alameda, Calif.) at 10 minutes post injection.

FIG. 7 shows gpD expression constructs. Plasmid vectors for prokaryotic expression of wild-type and recombinant forms of gpD are shown. (A) An E. coli codon-optimized (ECO) derivative of gpD was inserted into an ampicillin-selectable, pBR322-based pTrc expression plasmid, where it was placed under the transcriptional control of the high-level, regulatable Trc promoter. (B) A sequence coding for a high-affinity αvβ3 binding protein derived from the tenth fibronectin type III domain (3JCLI4) was inserted at the 3′ end of the gpD insert in the plasmid shown in (A), resulting in the generation of a construct that encoded a gpD-3JCLI4 fusion protein. (C) A matched dual expression plasmid set, capable of co-expressing wild-type gpD and the gpD-3JCLI4 fusion protein is shown. The ampicillin selection marker and pBR322-derived origin of plasmid replication in the plasmid shown in (A) were replaced with a spectinomycin resistance gene and a pCDF1-derived origin of replication, to generate the gpD-encoding plasmid shown on the left. (D) A second matched dual expression plasmid set, capable of co-expressing wild-type gpD and the gpD-3JCLI4 fusion protein is shown. In this case, the ampicillin selection marker and pBR322-derived origin of plasmid replication in the plasmid shown in (B) were replaced with a spectinomycin resistance gene and a pCDF1-derived origin of replication, to generate the gpD-3JCLI4-encoding plasmid shown on the right.

FIG. 8 shows co-expression of 3JCLI4-gpD and wild-type gpD yields intact phage particles. Lysogens of TOP10 cells containing gpD-deficient λ D1180 (Luc) were transformed with single plasmid vectors encoding either wild-type gpD (gpD; see FIG. 7A) or the recombinant gpD-3JCLI4 fusion protein (3JCLI4; see FIG. 7B). Alternatively, the cells were co-transformed with two plasmids, corresponding to the constructs shown in FIG. 7C (3JCLI4 DUAL) or FIG. 7D (CDF3JCLI4 DUAL); these paired constructs permitted the co-expression of wild-type and recombinant gpD in the same E. coli host cell. Following lysogen induction and cell lysis, phage particles were pelleted and subjected to cesium chloride density gradient purification. The large arrow denotes the characteristic λ phage band.

FIG. 9 shows co-expression of 3JCL14-gpD and wild-type gpD yields infectious phage particles. Lysogens of TOP10 cells containing gpD-deficient λ D1180 (Luc) were transformed with plasmid vectors encoding either wild-type gpD alone (gpD; see FIG. 7A) or with two plasmids, corresponding to the constructs shown in FIG. 7C (3JCLI4 DUAL) or FIG. 7D (CDF3JCLI4 DUAL). These paired constructs permitted the co-expression of wild-type and recombinant gpD in the same E. coli host cell. Following lysogen induction, CsCl-banding and dialysis, purified phage particles were titered on LE392 E. coli host cells. Results shown represent phage titers from three separate phage preparations; no appreciable amounts of infectious phage were recovered from lysogens that expressed only the gpD-3JCLI4 fusion protein (FIG. 7B).

FIG. 10 shows co-expression of 3JCLI4-gpD and wild-type gpD results in the generation of mosaic phage particles. Lysogens of TOP10 cells containing gpD-deficient λ D1180 (Luc) were transformed with plasmid vectors encoding either wild-type gpD alone (gpD) or with two plasmids that permitted the co-expression of wild-type and recombinant gpD in the same E. coli host cell (3JCLI4 DUAL, CDF3JCLI4 DUAL; see FIGS. 7-9). Following lysogen induction, CsCl-banding and dialysis, purified phage particles were titered on LE392 E. coli host cells. A total of 1×10⁹ plaque forming units (PFU) of each preparation was then loaded on a 20% SDS-PAGE gel and phage protein content was examined by immunoblot analysis, using a rabbit polyclonal antiserum directed against gpD.

FIG. 11 shows cellular immune responses to gp120 antigen are elicited by BALB/c mice immunized with bacteriophage lambda encoding HIV-1 gp120. Six week old female BALB/c mice were immunized intradermally (ID), via the tailbase, in groups of 4 with 1×10¹¹ PFU gpD or mChem (gp120) phage, or 100 μg gp120-encoding plasmid DNA as a positive control (all immunizations were in 50-100 μL total volume). Fourteen and 30 days following the initial immunization, mice in the phage and plasmid DNA groups were boosted with using a homologous prime-boost approach. Ten, 29, and 37 days following immunization, whole blood was used for tetramer staining with APC (allophyocyanin) conjugated H-2D_{d} tetramers loaded with the HIV-1 gp120 V3 peptide (RGPGRAFVTI; SEQ ID NO:66) (A). Mice were sacrificed at day 38 and splenocytes were used for gp120 tetramer staining, as above (B). Results are presented as mean values ±standard deviations.

FIG. 12 shows in vivo luciferase expression in BALB/c mice. Eight to twelve week old female BALB/c mice were injected intradermally (ID), via the tailbase, with either 1×10¹⁰, 5×10¹⁰, 1×10¹¹, or 5×10¹¹ PFU of gpD (luc) phage or 1×10¹¹ PFU of gpD (no luc) phage (A). Twenty-four hours later, mice were anesthesized and injected IP with luciferin. Nine minutes following luciferin injection, mice were imaged using the Xenogen IVIS system. Images were collected using an exposure time of 1 minute. The graph is representative of the average photon flux (photons/sec/cm²/sr) and standard deviation for each group. In vivo luciferase gene expression was found to be dose dependent in mice injected ID with gpD (luc) phage (A, n=3). In panel B, eight to twelve week old female BALB/c mice were injected intradermally (ID), via the tailbase, intramuscularly (IM) in the thigh, or subcutaneously (SQ), in groups of 8 with 1×10¹¹ PFU gpD (Luc) phage. Four mice were injected ID, IM, or SQ with 1×10¹¹ PFU phage that lacked the luciferase expression cassette (no Luc) as a negative control for each group (B). Mice were imaged, analyzed, and graphed as described for (A). The data show that the baseline level of measured photon flux in the assay is a little less than 1×10⁵ photons/sec/cm²/sr (see “no luc” group; A). Hence, a value of 1×¹⁰⁵ was applied as a cutoff in subsequent assays. In vivo luciferase gene expression was evident in mice injected ID with gpD (Luc) phage (B, n=8). In Panel C, eight to twelve week old female BALB/c mice were injected ID in groups of 8 with 1×10¹¹ PFU of either wild-type gpD phage, 3JCLI4 phage (phage containing a modified gpD protein bearing the 3JCL4 integrin-binding peptide), or chemerin (mChem) phage (phage containing a modified gpD protein containing a chemerin receptor-binding peptide). Four mice were injected with 1×10¹¹ PFU of phage that lacked the luciferase expression cassette (no Luc) as a negative control (C). Mice were imaged, analyzed, and graphed as described for (A). In vivo luciferase gene expression was found to be greater in animals injected with 3JCLI4 or chemerin targeted phage (C, n=8, p<0.05 one-way ANOVA, Tukey's post-test).

FIG. 13 shows in vivo luciferase expression in BALB/c mice injected with gpD (luc) phage is increased when mice are pre-immunized with bacteriophage lambda. Eight to twelve week old female BALB/c mice were first immunized IM with either 1×10¹¹ PFU of gpD (no luc) bacteriophage lambda (1) in 50 μL of suspension media or 50 μL of suspension media alone (no). Two weeks post-immunization, all mice were injected ID, via the tailbase, with 1×10¹¹ PFU of gpD (luc) phage. Twenty-four hours later, mice were imaged for luciferase expression, as described in FIG. 12. Results shown represent mean luciferase expression values, and the bars denote standard deviations of these means. There was a difference in in vivo luciferase gene expression between mice that were pre-immunized with bacteriophage lambda versus mice that were pre-immunized with suspension media alone (p<0.05, Student's two-tailed t-test; A). Sera from mice was collected two weeks post-immunization and analyzed by ELISA. Antibodies specific for bacteriophage lambda were detected in mice pre-immunized with bacteriophage lambda (B), as expected.

FIG. 14 shows in vivo luciferase expression in BALB/c mice injected with wild-type or 3JCL14-targeted phage is not affected by chloroquine treatment nor depletion of phagocytic cells by clodronate. In Panel A, eight to twelve week old female BALB/c mice were injected IP with 2 mg per mouse of chloroquine (shaded bars) or were left untreated (open bars). Two hours following chloroquine injection, 1×10¹¹ PFU of either wild-type gpD (luc) phage, 3JCLI4 (luc) phage, or 100 μg of gWIZ plasmid DNA (a plasmid vector encoding luciferase under the transcriptional control of the CMV promoter) was injected ID, via the tailbase. Twenty-four hours later, mice were imaged for luciferase expression, as described in FIG. 12. Results shown represent mean luciferase expression values, and the bars denote standard deviations of these means. No difference in in vivo luciferase gene expression was observed in animals treated with chloroquine prior to phage or DNA injection (A, n=4, p>0.1 for all comparisons, Student's two-tailed t-test). In panel B, eight to twelve week old female BALB/c mice were injected in groups of 4 with clodronate liposomes via a combined IP (200 μl) and ID (100 μl) route (shaed bars) or were left untreated (open bars). The liposomes were expected to selectively kill (and thus deplete) phagocytic cells. Forty-eight hours following clodronate liposome injection, 1×10¹¹ PFU of either wild-type gpD (luc) phage, 3JCLI4 (luc) phage, or 100 μg of gWIZ (luc) plasmid DNA was injected ID, via the tailbase. Mice were imaged and analyzed as described in (A). A decrease in in vivo luciferase gene expression in animals treated with clodronate liposomes prior to phage or DNA injection was observed (B, n=4, p>0.1 for all comparisons, Student's two-tailed t-test). After imaging, mice were sacrificed and splenocytes were stained for F4/80 (a cell surface marker for macrophages). Mice that received clodronate liposomes had a decrease in F4/80 positive splenocytes, as measured by flow cytometric analysis, showing that phagocytic cells were depleted in these mice (C). A representative staining profile for one control animal (left) and clodrontate-treated animal (right) is shown in the upper part of Panel C, and mean data from all animals are shown below, in histogram form (bars denote the standard deviation of these mean values).

FIG. 15 shows treatment of purified phage with benzonase eliminates in vivo luciferase expression in BALB/c mice injected with wild-type or 3JCLI4-targeted phage. Eight to twelve week old female BALB/c mice were injected in groups of 5 with 1×10¹¹ PFU of either wild-type gpD (luc) phage or 3JCLI4 (luc) phage. In Panel A, phage was either treated with 50 U of benzonase or buffer alone prior to ID injection, via the tailbase route. Twenty-four hours later, mice were imaged for luciferase expression, as described in FIG. 12. Results shown represent mean luciferase expression values, and the bars denote standard deviations of these means. Mice receiving benzonase treated phage did not have a detectable luciferase signal, whereas mice receiving untreated phage had strong luciferase expression (A). In Panel B, 6×10¹¹ PFU of gpD (luc) phage treated with 50 U or benzonase or buffer alone (50 μL total volume), and the treated phage were then re-titered on LE392 E. coli cells to determine if benzonase treatment affected phage viability. Phage titers were unaltered by benzonase treatment, indicating that benzonase treatment does not lead to loss of phage viability or damage to phage particles (B).

FIG. 16 shows in vivo luciferase expression in BALB/c mice injected with wild-type phage results from expression of between 1-5 μg of surface-bound lambda DNA. Eight to twelve week old female BALB/c mice were injected in groups of 8 with either (i) 1×10¹¹ PFU of wild-type gpD (luc) phage, (ii) 1×10¹¹ PFU of wild-type phage containing no mammalian expression cassette but physically mixed with 5 μg of purified lambda phage DNA that was isolated from the luciferase-encoding phage (R luc DNA), (iii) 5 μg of purified λ luc DNA, or (iv) 1 μg of λ luc DNA. Twenty-four hours later, mice were imaged for luciferase expression, as described in FIG. 12. Results shown represent mean luciferase expression values, and the bars denote standard deviations of these means. In vivo luciferase expression in BALB/c mice injected with gpD (luc) was similar to expression levels obtained from 1 and 5 μg of purified λ luc DNA alone, suggesting that purified phage may have between 1 and 5 μg of surface-bound phage DNA.

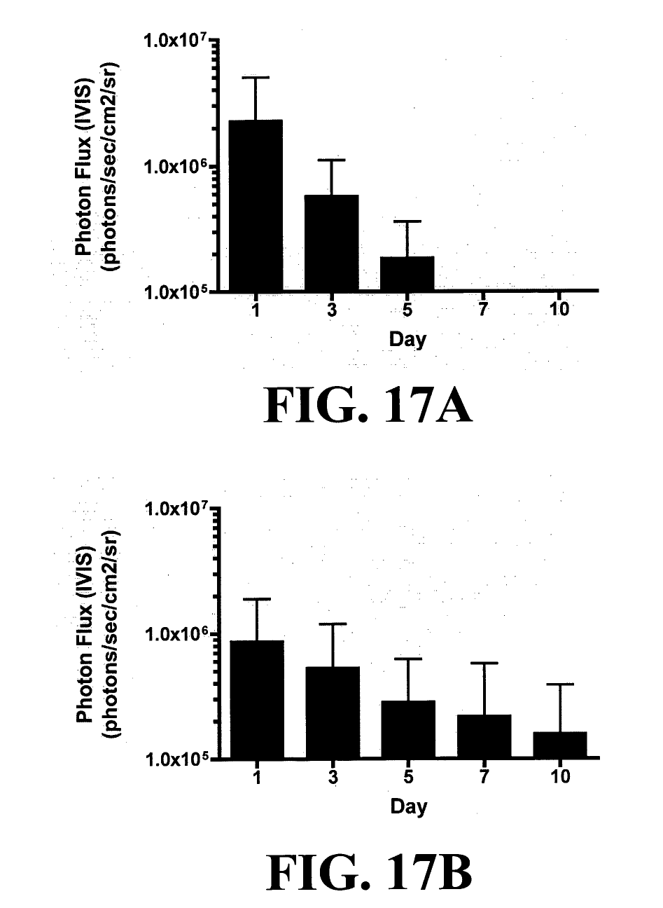

FIG. 17 shows in vivo luciferase expression in BALB/c mice injected with wild-type luciferase-encoding phage persists over a time-frame very similar to that for mice injected with purified λ luc DNA. Eight to twelve week old female BALB/c mice were injected in groups of 8 with either 1×10¹¹ PFU of wild-type gpD (luc) phage (panels A, C) or 5 μg of purified λ luc DNA (Panel B, D). One, 3, 5, 7, and 10 days following injection, mice were imaged for luciferase expression, as described in FIG. 12. Results shown in panels A and B represent mean luciferase expression values, and the bars denote standard deviations of these means. In vivo luciferase expression in mice injected with gpD (luc) is similar to expression levels obtained from mice injected with 5 μg purified λ luc DNA. Panels C and D represent an analysis of luciferase expression in each of the 8 experimental animals per group that are represented in the summary histograms (panels A and B). The thick line denotes the cutoff of the assay.

### DETAILED DESCRIPTION

The present invention may be understood more readily by reference to the following detailed description of the invention and the Examples included therein and to the Figures and their previous and following description.

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance may or may not occur, and that the description includes instances where said event or circumstance occurs and instances where it does not.

Ranges can be expressed herein as from “about” one particular value, and/or to “about” another particular value. When such a range is expressed, another embodiment includes from the one particular value and/or to the other particular value inclusive of endpoints. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that these data, represent endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

“Subject” includes, but is not limited to, animals, plants, bacteria, viruses, parasites and any other organism or entity that has nucleic acid. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The subject may to an invertebrate, more specifically an arthropod (e.g., insects and crustaceans). The term does not denote a particular age or sex. Thus, adult and newborn subjects, as well as fetuses, whether male or female, are intended to be covered. A patient refers to a subject with a disease or disorder. The term “patient” includes human and veterinary subjects.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that, while specific reference of each various individual and collective combinations and permutation of these compounds may not be explicitly disclosed, each is specifically contemplated and described herein. For example, if a particular modification is disclosed and discussed and a number of modifications are discussed, specifically contemplated is each and every combination and permutation of the modifications that are possible unless specifically indicated to the contrary. Thus, if a class of molecules A, B, and C are disclosed as well as a class of molecules D, E, and F and an example of a combination molecule, A-D is disclosed, then, even if each is not individually recited, each is individually and collectively contemplated, meaning combinations A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Provided herein is an antigen delivery system, including a vaccine delivery system, comprising a modified lambda phage and a phage encoded antigen. As used herein, “antigen delivery system” refers to a composition, wherein the composition can be combined with an identified antigen and wherein the composition can deliver the antigen to antigen presenting cells of a subject for the purpose of eliciting an antigenic response in the subject. As used herein “antigen” refers to any substance that stimulates the production of antibodies or expansion of specific T cell clone(s). The term “immunogen” refers to any substance or organism that provokes an immune response (produces immunity) when introduced into the body. It is understood that the antigen of the provided invention can also be an immunogen. As used herein “antigen presenting cell” refers to a cell that carries on its surface antigen bound to MHC Class I or Class II molecules and presents the antigen in this context to T-cells. This can include macrophages, endothelium, dendritic cells and Langerhans cells of the skin, as well as other cell types under certain circumstances. As disclosed and described herein, the provided antigen delivery system can be used to elicit humoral immunity or cellular immunity. Thus, the herein provided modified phage can be used to elicit a T-cell response.

Also provided is a gene delivery system comprising a modified λ phage and a phage-encoded gene of interest. The lambda phage of the provided delivery systems can comprise a surface polypeptide modified to target a selected cell (e.g., antigen-presenting cells). As used herein, “modified” refers to any alteration(s) that affects either form or function. For example, the modifications to lambda phage vectors provided herein include modifications designed to increase phage survival in the human host and enhance phage binding to mammalian cells. As used herein, “surface polypeptide” refers to a native or heterologous polypeptide that is expressed by and exposed on the phage surface. It is understood that a molecule can be displayed on the surface of the phage by conjugating the molecule to a surface polypeptide. As used herein, relative terms such as “increase” or “enhance” refer to a change as compared to a control. For example, enhanced uptake of a modified phage refers to an increase as compared to a non-modified wild-type phage. “Increased” or “enhanced” refers to a statistically significant increase over control.

Lambda has two identified platforms for peptide display. The surface polypeptide of the provided lambda phage can be gpD or gpV. The gpD and gpV surface polypeptides can have the amino acid sequence of SEQ ID NO:39 and SEQ ID NO:40, respectively. Modifications within the gpD or gpV amino acid sequences can include insertions, deletions, truncations, substitutions (including conservative amino acid substitutions). Thus, provided herein are modified and variant forms of gpD and gpV. The gpD surface polypeptide can be encoded, for example, by the nucleic acid sequence of SEQ ID NO:52 The gpD surface polypeptide can further be encoded by a codon-optimized version of SEQ ID NO:52 using techniques and procedures readily available to one skilled in the art (Gao, W. et al. 2004. Biotechnol Prog 20:443-448; Fuglsang, A. 2004. Protein Expr Purif 31; 247-249; Bradel-Tretheway, B. G. et al. 2003. J Virol Methods 111:145-156; Grosjean, H. and Fiers, W. 1982. Gene 18:199-209). Thus, the gpD surface polypeptide can be encoded by the nucleic acid sequence of SEQ ID NO: 1. The gpV surface polypeptide can be encoded, for example, by the nucleic acid sequence of SEQ ID NO:53. The gpV surface polypeptide can be further encoded by a codon-optimized version of SEQ ID NO:53. Further provided herein are nucleic acids that encode the modified gpD and gpV amino acid sequences.

As disclosed herein, the modified surface polypeptide of the invention can be a homologue derived from a closely related phage. Thus, the surface polypeptide can be gpShp from lambda-like phage 21, which shares 49% amino acid identity to gpD (Wendt J L, Feiss M. 2004. Virology 326:41-6). The known gpShp surface polypeptide has the amino acid sequence of SEQ ID NO:41 and can be encoded, for example, by the relevant nucleic acid sequence contained within SEQ ID NO:42. As provided herein, the gpshp encoding nucleic acid or gpShp amino acid can be modified.

It is understood that one way to define any variants, modifications, or derivatives of the disclosed genes and proteins herein is through defining the variants, modification, and derivatives in terms of homology to specific known sequences. As used herein, “homologue” refers to a polypeptide or nucleic acid with homology to a specific known sequence. Specifically disclosed are variants of the nucleic acids and polypeptides herein disclosed which have at least 40, 41, 42, 43, 44, 45, 46, 47, 48, 49, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated or known sequence. Those of skill in the art readily understand how to determine the homology of two proteins or nucleic acids. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. These references are incorporated herein by reference in their entirety for the methods of calculating homology.

The same types of homology can be obtained for nucleic acids by, for example, the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

The surface polypeptide can further be a chimeric polypeptide comprising portions of gpD, gpV and/or gpShp, for example, the N-terminus of gpD or gpV and the C-terminus of gpShp or any other combination thereof.

Provided herein are modifications and treatments to enhance phage targeting and entry into antigen presenting cells or other target cells. The phage surface polypeptides are modified to enhance targeting to antigen presenting cells, to enhance uncoating, to enhance phage escape from endosomes, to enhance phage transport across membranes, to enhance uptake, or any combination of two or more of any of the modifications taught herein.

Provided herein are modifications or combinations of modifications to lambda phage surface polypeptides designed to enhance the targeting of the phage to antigen-presenting cells or other target cells. The modifications can comprise the addition of one or more polypeptides that target specific receptors on antigen presenting cells.

Chemerin receptor (ChemR23) is a G-protein coupled receptor structurally related to chemokine receptors that is expressed on immature dendritic cells and macrophages (Wittamer, V., et al. 2003. J Exp Med 198:977-85; Wittamer, V., et al. 2004. J Biol Chem 279:9956-62). Prochemerin is the natural precursor ligand of chemerin receptor and is cleaved at the carboxy terminus to create active chemerin. A nonapeptide at the carboxy terminus of chemerin was identified as sufficient to retain both the binding and agonist activity of full-length chemerin. Although very little is known about the function of chemerin receptor on APC, the almost exclusive expression of this receptor on DCs and macrophages makes it an attractive target for DC specific vectors.

Thus, the modified surface polypeptide of the provided delivery system can comprise a polypeptide that binds the chemerin receptor. The polypeptide can be chemerin. The polypeptide can be a chemerin-like peptide. The modified surface polypeptide can comprise chemerin fragments, including nonapeptides. The modified surface polypeptide can comprise the amino acid sequence of SEQ ID NO:2 (YFPGQFAFS) or SEQ ID NO:3 (YFLPGPFAFS) in the presence or absence of one or more conservative amino acid substitutions.

The integrin αᵥβ₃ (CD51/CD61) is expressed in low levels on dendritic cells and is important for dendritic cell migration to local lymph nodes (Weiss, J. M., et al. 2001. J Exp Med 194:1219-29). Fibronectin is a component of the extracellular matrix and known to bind with αvβ3. The tenth fibronectin type III domain (FNfn10) contains the binding site (an RGD motif) and can be used to select for high affinity binding peptides of αvβ3. One identified sequence, FNfn10-3JCLI4, binds with high specificity and affinity to αvβ3 as compared to the wild type FNfn10 sequence (Richards, J., et al. 2003. J Mol Biol 326:1475-88). The 3JCLI4 amino acid sequence (for example, the amino acid sequence of SEQ ID NO:4) has an affinity equivalent to an αvβ3 monoclonal antibody but is better suited to peptide display due to its small size, stability, and lack of cysteines.

Thus, the modified surface polypeptide of the provided delivery system can comprise a polypeptide that binds an integrin. The integrin binding polypeptide of the provided delivery system can be derived from FNfn10. The integrin binding polypeptide of the provided delivery system can comprise Fnfn10-3JCL14. The modified surface polypeptide can comprise the amino acid sequence of SEQ ID NO:4 in the presence or absence of one or more conservative amino acid substitutions.

The integrin binding polypeptide of the provided delivery system can comprise one or more snake venom disintegrin, or a variant or fragment thereof. Snake venom disintegrins are a family of small peptides (˜50-70 amino acids generally) that inhibit the adhesive activities of mammalian integrins. Disintegrins include but are not limited to flavoridin, lachesin, kistrin, and echistatin. These molecules have the ability to interfere with platelet aggregation by interacting with the platelet integrin, αIIbβ3. The snake venom disintegrins typically include an RGD motif flanked by multiple disulfide bonds and have the ability to bind integrins from a wide range of mammalian species including mice, rats, pigs and humans.

The polypeptide comprising a snake-venom disintegrin can comprise flavoridin or a variant or fragment thereof. Flavoridin is a monomeric disintegrin (Bilgrami, S., et al. 2004. J Mol Biol 341:829-37), which binds αᵥβ₃ with subnanomolar affinity (Ki=0.06 nM), but binds other integrins much less tightly (Ki=1.4 nM for α₈β₁ and 4.2 nM for α_{3/5/v}β₁) (Thibault, G. 2000. Mol Pharmacol 58:1137-45). For example, a modified surface polypeptide can, thus, comprise the amino acid sequence SEQ ID NO: 5 or 6 in the presence or absence of one or more conservative amino acid substitutions.

The polypeptide comprising a snake-venom disintegrin can comprise lachesin or a variant or fragment thereof. Lachesin is a disintegrin that preferentially binds to αᵥβ₃ as compared to other integrins. Its IC50 for blocking the vitronectin/αᵥβ₃ interaction is 4 nM, whereas its IC50 for blocking the fibrinogen/GPIIB-IIIa interaction is 19 nM (Scarborough, R. M., et al. 1993. J Biol Chem 268:1058-65). Thus, the modified surface polypeptide can comprise the amino acid sequence SEQ ID NO: 7 or 8 in the presence or absence of one or more conservative amino acid substitutions.

The polypeptide comprising a snake-venom disintegrin can comprise kistrin or a variant or fragment thereof. Kistrin preferentially binds to β₃-containing integrins (Leyton, L., et al. 2001. Curr Biol 11:1028-38). A fragment from the integrin-binding loop of kistrin can be used. Thus the modified surface polypeptide can comprise the amino acid sequence SEQ ID NO: 9 in the presence or absence of one or more conservative amino acid substitutions.

The polypeptide comprising a snake-venom disintegrin can be comprised of echistatin (Jones, J. I., et al. 1996. Natl Acad Sci USA 93:2482-7; Juliano, D., et al. 1996. Exp Cell Res 225:132-42; Legler, D. F., et al. 2004 Eur J Immunol 34:1608-16; Marcinkiewicz, C., et al. 1997. Blood 90:1565-75; Scheibler, L., et al. 2001. Biochemistry 40:15117-26; Tselepis, V. H et al. 1997. J Biol Chem 272:21341-8; Wiedle, G., et al. 1999. Cancer Res 59:5255-63) or a variant or fragment thereof. Thus, the modified surface polypeptide can comprise the amino acid sequence of SEQ ID NO:43 in the presence or absence of one or more conservative amino acid substitutions.

Expression of these disintegrin comprising polypeptides can be facilitated by the use of E. coli strains that possess an oxidizing cytoplasmic environment, such as ORIGAMI™ cells (Novagen, San Diego, Calif.).

Cell surface receptors on antigen-presenting cells or other target cells can be targeted using antibody molecules attached to the phage surface polypeptide. This can be achieved genetically via biotin-avidin bridges or via IgG-binding motifs.

Biotin has a extremely high affinity for avidin or streptavidin, as do several certain biotin-like polypeptides, several of which contain the amino acid sequence HPQ. The (strept)avidin/biotin binding motif has been used successfully in the past for mammalian receptor targeting and gene delivery by filamentous phage (Larocca, D., et al. 1998. Hum Gene Ther 9:2393-9). Phage surface polypeptides can be modified to include (strept)avidin-binding motifs. The amino acid sequence of SEQ ID NO: 12, for example, permits in vitro biotinylation using purified BirA, or in vivo biotinylation in E. coli strains that express BirA (Cloutier, S. M., et al. 2000. Mol Immunol 37:1067-77; Lesley, S. A. and D. J. Groskreutz. 1997. J Immunol Methods 207:147-55). These protein fusion can then allow receptor targeting by biotinylated antibodies, or other biotinylated ligands, bound non-covalently to lambda phage particles through an avidin or streptavidin bridge.

Either biotin- or (strept)avidin-binding moiety can be incorporated into the phage surface polypeptide. Thus, the modified surface polypeptide of the provided delivery system can comprise a (strept)avidin-binding moiety. This moiety can be biotin, biotin-like, or a derivative of biotin. The modified surface polypeptide can comprise the amino acid sequence SEQ ID NO: 10, 11, or 12 or fragments or variants thereof, including the sequences with one or more conservative amino acid substitutions. The delivery system comprising an avidin-binding moiety in the surface polypeptide further comprises an avidin-linked polypeptide that targets an endocytosing receptor on the antigen-presenting cell. The avidin-linked polypeptide can be an antibody or fragment thereof that binds endocytic receptors such as, for example, DEC205 (CD205), Langerin (CD207), DC-SIGN (CD209) or related C-type lectin receptors, or FcγR1 (CD64). The avidin-linked polypeptide can also be transferrin, mannose, or a derivative thereof, that binds an endocytosing receptor. Conversely, when the delivery system comprises a biotin-binding moiety in the surface polypeptide, the delivery system can further comprise a biotin-linked polypeptide that targets endocytic receptors. DEC205, Langerin, DC-SIGN and CD64 are all endocytosing receptors which are expressed on dendritic cells and/or Langerhans cells of the skin, as well as, in some cases, additional antigen-presenting cell-types such as macrophages (Valladeau, J. et al. 2000. Immunity 12:71-81; Jiang, W. et al. 1995. Nature 375:151-155; Geijtenbeek, T. B. et al. 2000. Cell 100:575-585; Aderem, A., and Underhill, D. M. 1999. Annu Rev Immunol 17:593-623). Previous studies have shown that antigen-targeting to the DEC205 or CD64 receptor can lead to enhanced immune responses (Bonifaz L. C., et al. 2004. J Exp Med 199:815-824; Liu C., et al. 1996. J. Clin Invest 98:2001-2007), while DC-SIGN has been implicated in the binding and entry of several viruses to dendritic cells, including Hepatitis C Virus and HIV-1 (Pohlmann, S. et al. 2003. J Virol 77:4070-4080; Geijtenbeek, T. B. et al. 2000. Cell 100:587-597) and Langerin has been shown to be involved in antigen presentation to T cells (Hunger, R. E., et al. 2004. J Clin Invest 113:701-708).

The modified surface polypeptide of the provided delivery systems can comprise an IgG-binding motif or an IgA-binding motif. As used herein, “IgG-binding motif” or “IgA-binding motif” refers to any molecule, or portion of a molecule, that selectively binds IgG or IgA, respectively. The IgG- or IgA-binding motifs can comprise IgG or IgA binding polypeptides expressed by a bacteria or virus.

The IgG-binding polypeptide can comprise, for example, Staphylococcus protein A or Streptococcus protein G. The IgG-binding polypeptide can comprise a viral IgG Fc-binding protein encoded by herpesviruses such as HSV-1, VZV, or HCMV. Examples include glycoprotein E, gpUL119-118 and TRL11/IRL11. The IgG-binding morif can comprise an IgG antibody or fragment thereof that binds endocytic receptors of antigen presenting cells such as DEC205, DC-SIGN, Langerin, or CD64/FcγR1.

The B domain of protein A (Z) from Staphylococcus and the C2 domain of protein G from Streptococcus contain binding motifs that recognize the IgG constant regions from most mammalian species. A synthetic derivative of the B domain (Z) has been fused to multiple gene delivery vectors, including adenovirus, and used to mediate gene delivery through receptor specific antibodies (Henning, P., et al. 2002. Hum Gene Ther 13:1427-39; Ohno, K., et al. 1997. Nat Biotechnol 15:763-7; Volpers, C., et al. 2003. J Virol 77:2093-104). Therefore, gpD fused to known IgG binding motifs can be used to target dendritic cells. The modified surface polypeptide can comprise a polypeptide derived from the C2 domain of protein G from Streptococcus. The modified surface polypeptide can comprise the amino acid sequence of SEQ ID NO: 15 in the presence or absence of one or more conservative amino acid substitutions.

Two synthetic derivatives of the B domain (Z) are often fused together to increase theoretical IgG binding, but recombination has been known to occur between multiple repeating Z domains, even in RecA negative strains (Nilsson, B., et al. 1987. Protein Eng 1: 107-13). Therefore, a synthetic ZZ gene is provided with the first Z sequence being the native sequence, and the second Z sequence being codon optimized for expression in E. coli. This decreases homology with the first Z domain, thereby reducing the subsequent likelihood of recombination. The modified surface polypeptide can comprise derivatives of the B domain (Z). The modified surface polypeptide can comprise amino acid sequence SEQ ID NO: 13 with one or more conservative amino acid substitutions. An example of a nucleic acids encoding the modified surface polypeptide comprises the nucleic acid of SEQ ID NO: 14.

The surface polypeptide of the delivery systems can comprise an IgA-binding motif. The IgA-binding polypeptide can comprise, for example, an IgA-binding polypeptide expressed by Staphylococcus, e.g., members of the M protein family, including Arp4 and Sir22 (reviewed in: Woof, J. N. 2002. Biochem Soc Trans 30:491-494). The IgA binding motif can comprise an IgA antibody or fragment thereof. The IgA antibody or fragment can bind an endocytosing receptor such as, for example, DEC205, DC-SIGN, Langerin, or CD64/FcγR1. IgA-binding proteins encoded by bacterial species include several Streptococcal proteins which interact with the Fc component of IgA, including the b protein from group B streptococci as well as the Sir22 and Arp4 proteins from Streptococcus pyogenes (reviewed in: Woof, J. N. 2002. Biochem Soc Trans 30:491-494). These microbial proteins also interfere with interaction between the mammalian cellular IgA receptor (CD89) and the Fc portion of IgA (reviewed in: Woof, J. N. 2002. Biochem Soc Trans 30:491-494)

The modified surface polypeptide of the delivery system can comprise an immuno-conjugate or antibody fusion polypeptide, wherein the antibody fusion polypeptide comprises a single chain antibody (scFv) directed to an endocytosing receptor such as, for example, DEC205, DC-SIGN, Langerin, or CD64/FcγR1, conjugated or fused to a phage surface polypeptide. The addition of single-chain antibody molecules to gpD, for example, requires the formation of disulfide bonds or a peptide linkage. Lambda is optimally assembled in the E. coli cytoplasm, while ScFv needs to be exported to the periplasm for disulfide bond formation (Venturi, M., et al. 2002. J Mol Biol 315:1-8). Successful disulfide bond formation of ScFv has been described in lambda grown on the E. coli strain BM25.8 (Gupta, A., et al. 2003. Advances in Virus Research 60:421-67). Therefore, gpD-scFv fusions can be expressed in an E. coli strain which possesses an oxidizing cytoplasm for disulfide bond formation, e.g., ORIGAMI™ strains from Novagen/Embiosciences (San Diego, Calif.). The gpD-scFv can then be combined with lambda phage in two ways. First, lambda lysogens of interest can be created in the E. coli strains that contain the gpD-scFv expression plasmids. Alternatively, the gpD-scFv proteins can be purified and combined with gpD-deficient lambda phage particles in a cell-free system as described by Sternberg and Hoess (Sternberg, N., and R. H. Hoess. 1995. Proc Natl Acad Sci USA 92:1609-13).

The lambda phage of the provided delivery systems can comprise a mutation in a coat protein. As used herein, “mutation” refers to an alteration in the amino acid or nucleotide sequence that affects biological activity. Examples of mutations can include substitutions, deletions or insertions of amino acids or nucleotides. The mutated coat protein can be a lambda E capsid protein (including, for example, the amino acid sequence of SEQ ID NO:44). Thus, the lambda E capsid protein can comprises, for example, an E to K substitution at residue 158 of SEQ ID NO:44 (SEQ ID NO:45), as described in the long-circulating Argo1 and Argo2 mutants of bacteriophage lambda by Merril (Merril, C. R. et al. 1996. Proc Natl Acad Sci USA 93:3188-3192).

The surface polypeptide of the provided delivery systems can be modified to enhance phage uncoating in the mammalian cell. Provided herein is a modified lambda phage comprising a surface polypeptide fusion protein, wherein the fusion protein destabilizes a viral capsid. For example, the polypeptide of the fusion protein provided herein can comprise a PEST or ubiquitination motif.

As used herein, “PEST motif” refers to a region of a polypeptide rich in the amino acids proline (P); glutamic acid (E); serine (S); or threonine (T) that is associated with rapidly degraded proteins. The pore-forming protein listeriolysin O (LLO), secreted by Listeria monocytogenes, was shown to contain a PEST-like sequence that is essential for the virulence and intracellular compartmentalization of this pathogen. Mutants lacking the PEST-like sequence enter the host cytosol but are subsequently permeabilized and killed by the host cell. LLO lacking the PEST-like sequence accumulate in the host-cell cytosol, suggesting that this sequence targets LLO for degradation. Thus, in order to destabilize a viral capsid, the surface polypeptide provided herein can comprise a PEST motif. The surface polypeptide can comprise a PEST motif from listeriolysin 0 (LLO) protein, related bacterial lysins, or NF-□B1 p105. The PEST motif can comprise amino acid sequence of SEQ ID NO: 16, 17, 18, or 19 in the presence or absence of one or more conservative amino acid substitutions. The provided sequences can enhance degradation of the phage capsid in mammalian cells and thereby enhance DNA extrusion in mammalian cells without affecting stability in bacteria (Lang, V., et al. 2003. Mol Cell Biol 23:402-13; Lety, M. A., et al. 2002. Mol Microbiol 46:367-79; Lety, M. A., et al. 2001. Mol Microbiol 39:1124-39; Salmeron, A., et al. 2001. J Biol Chem 276:22215-22).

In order to destabilize a viral capsid, the polypeptide of the fusion protein provided herein can comprise a ubiquitination motif. The ubiquitination motifs may comprise motifs from RNA virus proteases (Lawson, T. G., et al. 1999. J Biol Chem 274:9871-80; Losick, V. P., et al. 2003. Virology 309:306-19). The ubiquitination motifs can comprise SEQ ID NO:20 or 21 in the presence or absence of one or more conservative amino acid substitutions. The ubiquitination motifs can be derived from mammalian ubiquitin sequences (Rodriguez, F., et al. 1998. J Virol 72:5174-81). The ubiquitination motifs can comprise SEQ ID NO:22 in the presence or absence of one or more conservative amino acid substitutions. These sequences will enhance the proteasomal targeting of phage particles and may thereby enhance DNA delivery to the nucleus. Ubiquitination and proteasomal targeting of a mammalian virus enhances viral DNA delivery to the nucleus (Ros, C. and C. Kempf. 2004. Virology 324:350-60).

The surface polypeptide of the provided delivery system can be modified to enhance escape of phage vectors from endosomes. Endosome-disruptive peptides include pH-dependent fusogenic peptides derived from the influenzavirus HA protein (Han, X., et al. 2001. Nat Struct Biol 8:715-20; Plank, C., et al. 1994. J Biol Chem 269:12918-24; Prchla, E., et al. 1995. J Cell Biol 131:111-23; Skehel, J. J., et al. 2001. Biochem Soc Trans 29:623-6; Wadia, J. S., et al. 2004. Nat Med 10:310-5).

Also provided are peptides which combine endosomal escape and degradative properties. Thus, the modified surface polypeptide can comprise an HA2 or GALA-INF3 polypeptide. The modified surface polypeptide can comprise SEQ ID NO:23 or 24 in the presence or absence of one or more conservative amino acid substitutions.

Another such peptide is the extended PEST motif (including, for example, a motif comprising the amino acid sequence of SEQ ID NO: 16 or 17) from the listeriolysin 0 protein. This domain overlaps a domain that is essential for phagosomal escape of Listeria monocytogenes (Decatur, A. L. and D. A. Portnoy. 2000. Science 290:992-5; Lety, M. A., et al. 2002. Mol Microbiol 46:367-79; Lety, M. A., et al. 2001. Mol Microbiol 39:1124-39).

Provided herein is the use of polypeptides to selectively promote the uptake of phage vectors into antigen-presenting cells or other target cells, via pathways which are independent of the normal phagocytic and/or endosomal delivery pathways. These delivery approaches avoid the acidic endosomal environment and the lysosomal compartment—thereby promoting more efficient gene expression and enhanced immune responses to phage-encoded genes. Thus, the surface polypeptide of the provided delivery system can be modified to promote non-phagocytic/non-endosomal membrane transport.

In order to promote non-phagocytic/non-endosomal membrane transport, the modified surface polypeptide of the provided delivery system can comprise a basic peptide transduction domain (PTD). Examples of PTDs include PTDs from Antennipedia, HIV-1 Tat, Pep 1, and derivatives thereof, which typically comprise the sequence RKKRRQRRR (SEQ ID NO:38). PTDs have been shown to enhance phage delivery into mammalian cells (Eguchi, A., et al. 2001. J Biol Chem 276:26204-10, Console, S., et al. 2003. J Biol Chem 278:35109-14; Derossi, D., et al. 1994. J Biol Chem 269:10444-50; Morris, M. C., et al. 2001. Nat Biotechnol 19:1173-6; Park, J., et al. 2002. J Gen Virol 83:1173-81; Schwarze, S. R., et al. 1999. Science 285:1569-72). The surface polypeptide can comprise a PTD in combination with any of the other modifications provided herein. The PTD can be derived from Antennipedia, HIV-1 Tat, or Pep1. The PTD can comprise amino acid sequence of SEQ ID NO:25, 26 or 27.

In order to promote non-phagocytic/non-endosomal membrane transport, the modified surface polypeptide can comprise the E. coli pilus protein FimH. FimH binds to CD48, a surface receptor found on macrophages, and permits bacterial entry via lipid rafts. The result is that FimH-bearing E. coli strains enter the cytosol of mammalian cells via a pathway that does not involve endosomes, acidification or exposure to oxidative attack (Baorto, D. M., et al. 1997. Nature 389:636-9). The modified surface polypeptide of the provided delivery system can therefore comprise E. coli pilus protein, FimH. The modified surface polypeptide of the provided delivery system can therefore comprise the amino acid sequence of SEQ ID NO:28 in the presence or absence of one or more conservative amino acid substitutions. The modified surface polypeptide of the provided delivery system can therefore be encoded, for example, by the nucleotide sequence of SEQ ID NO:29.

In order to promote non-phagocytic/non-endosomal membrane transport, the modified surface polypeptide can comprise the leucine-rich repeat (LRR) domain of Leishmania parasite surface antigen-2 (PSA-2). A recombinant LRR domain from PSA-2 of Leishmania infantum binds to macrophages via the complement receptor 3 (CR3, Mac-1) (Kedzierski, L., et al. 2004. J Immunol 172:4902-6). This interaction occurs in both mouse and human macrophages and has been suggested to be important for the invasion of host cells by the Leishmania parasite (Kedzierski, L., et al. 2004. J Immunol 172:4902-6). CR3-dependent entry by other microbial pathogens results in increased bacterial survival, in part because this entry pathway fails to trigger the oxidative burst in phagosomes (Hellwig, S. M., et al. 2001. J Infect Dis 183:871-9; Rosenberger, C. M. and B. B. Finlay. 2003. Nat Rev Mol Cell Biol 4:385-96). The modified surface polypeptide of the provided delivery system can therefore comprise a leucine-rich repeat (LRR) motif of Leichmania parasite surface antigen-2 (PSA-2). The modified surface polypeptide of the provided delivery system can therefore comprise the amino acid sequence of SEQ ID NO:30. The modified surface polypeptide of the provided delivery system can therefore be encoded by a nucleic acid sequence comprising, for example, the nucleotide sequence of SEQ ID NO:31.

In order to promote non-phagocytic/non-endosomal membrane transport, the modified surface polypeptide of the provided delivery system can comprise a peptide or protein which binds to CD13 (aminopeptidase N), or another cell surface molecule which can become localized to caveolae and/or lipid rafts. A short peptide that includes the sequence motif NGR has been shown to bind to CD13 (Pasqualini, R. et al. 2000. Cancer Res 60:722-727), and can used to direct mammalian virus vectors to bind to this receptor (Grifman, M. et al. 2001. Mol Ther 3:964-975). This can be important for virus entry into mammalian cells, since CD13 has been shown to be a receptor for coronaviruses and to be involved in allowing coronaviruses to access the caveolar region, by virtue of its ability to colocalize with caveolae (Nomura, R. et al. 2004. J Virol 78:8701-8708). In fact, several viruses appear to enter mammalian cells via caveolae, include large enveloped viruses (filoviruses) and small non-enveloped viruses (SV40) (Empig, C. J., and Goldsmith, M. A. 2002. J Virol 76:5266-5270; Anderson, H. A. et al 1996. Mol Biol Cell 7:1825-1834; Pelkmans, L., et al. 2001. Nat Cell Biol 3:473-483). The modified surface polypeptide of the provided delivery system can therefore comprise a CD13-binding peptide. The modified surface polypeptide of the provided delivery system can therefore comprise the amino acid sequence of SEQ ID NO:46, 47, 48 or 49.

As disclosed herein, the presence of DNA on the outer surface of phage particles enhances lambda infectivity DNA may bind to lambda particles due to local charge interactions. Thus, the modified surface polypeptide of the provided delivery system can comprise a positively charged polypeptide, or plurality thereof. Lysine, arginine, and histidine are basic amino acids that carry a positive charge at physiological pH. Thus, as an example, the polypeptide can be an oligolysine, oligoarginine, or oligohistidine, or combinations thereof, wherein the polypeptide can comprise, for example, 1, 2, 3, 4, 5, 6, 7, 8, 9, 10, 11, 12, 13, 14, 15, 16, 17, 18, 19, 20, or more positively charged amino acids.

There are between 405 and 420 copies of gpD per phage, allowing for high copy numbers of a displayed peptide. Therefore a peptide, such as a modified polypeptide disclosed herein, incorporated in the gpD protein could be expressed 405-420 times on the surface of each particle, increasing the likelihood of the peptide forming a stable interaction with its target. In some cases this may be too high of a ligand density for functional binding or this high density of peptide may interfere with capsid formation. The peptide density could be lowered using a mosaic phage approach, wherein a combination of modified and unmodified surface proteins are expressed by the phage.

Thus, provided is an antigen delivery system or gene delivery system comprising a modified lambda phage and a phage encoded antigen or gene of interest, wherein the lambda phage comprises more than one surface polypeptide and wherein at least one of the surface polypeptides is modified to target an antigen-presenting cell. Thus, the ratio between the modified surface polypeptide and the unmodified surface polypeptide can be about 0:420 to 420:0, or any ratio in between. For example, the ratio between the modified surface polypeptide and the unmodified surface polypeptide can be about 1:419, 5:415, 10:410, 20:400, 50:370, 100:320, 210:210, 320:100, 370:50, 400:20, 410:10, 415:5, or 419:1. The phage can also comprise two or more modified surface polypeptides. Thus, any ratio is possible so long as the total number of surface polypeptides does not exceed about 405 to 420 polypeptides per particle.

The phage can also comprise combinations of modifications in which a short peptide is displayed at high copy (multivalent display) and a second, larger peptide is displayed at low or intermediate copy (oligovalent display). Examples of such display approaches include peptides with high binding affinity (such as scFvs), which can be displayed on oligovalent fashion, and peptides with low or intermediate binding affinity, or specific charge properties, which can be displayed at high copy number, to increase avidity (or to alter global surface charge on the phage particle).

The ratio of surface polypeptides can be adapted using standard methods known in the art. For example, combinations of high- and low-copy origins of replication can be used, as exemplified herein. As another example, programmed frameshifting elements or suppressable termination codons (in combination with an appropriate suppressor tRNA) can be used such that the nucleic acid encoding the surface polypeptide for which lower expression is desired is expressed in stoichiometrically titered levels, relative to the wild-type protein. Thus, the modified and unmodified surface polypeptides can be expressed in cis or in trans.

The ratio of surface polypeptides can be regulated by expressing 2 or more such peptides using plasmids with transcriptional promoters of different strength or inducible promoter elements (such as native or synthetic IPTG-inducible promoters, T7 promoter-based vectors, the phoA promoter, and other transcriptional regulatory elements known to those practiced in the art).

The ratio of surface polypeptides can be regulated by expressing 2 or more such peptides using genes that contain introduced suppressible translational stop codons, ribosome shifting sequencing, or codon sequences that have been altered to be either more or less optimal for E. coli host.

The ratio of surface polypeptides can also be regulated by expressing 2 or more such peptides in the same E coli host cells, using plasmids that are maintained at different copy numbers. Plasmid replicons that can be carried by plasmid vectors and be used in this fashion include elements that result in low copy number plamis maintenance (such as the pSC101 replicon and the F episome replicon), medium copy number plasmid maintenance (such as the p15A replicon contained in pACYC, the pMB origin contained in pBR322-, the CloDF13 replicon, and the colE1 replicon) and high copy number plasmid maintenance (such as the modified pMB1 element contain in pUC, and the pKN402 replicon contained in pMOB45). Use of origin elements from different complementation groups will permit simultaneous expression of many different modified surface polypeptides within a single E. coli host cell.

The display of multiple different surface polypeptides on the same lambda phage particle will result in the generation of mosaic phage. Mosaic derivatives of filamentous phages, including Type3+3 phagemid systems for low copy number display on the pIII protein, and Type 88 vectors for multivalent display on the major coat protein, pVIII, are known to those skilled in the art. These systems rely upon co-expression of wild-type and recombinant pVIII from a single phage genome (Type 88 vectors), or expression of wild-type pIII by helper phage, and recombinant pIII by a phagemid (Type3+3 vectors). As disclosed herein, mosaic phage can be generated using a coat-protein deficient phage genome that is complemented in trans, using one or many plasmid expression vectors to achieve a very high diversity of different possible combinations of surface polypeptides on the surface of each phage particle that is produced. The present system also has improved biocontainment and safety. This is because of the use of a replication-defective lambda host (containing a deletion of the phage gpD gene), combined with the use of a non-homologous, codon-altered gpD cassette in the complementing plasmid expression construct(s). This minimizes any potential for recombination between the lambda genome and the complementing plasmid.

The term “peptide”, “polypeptide”, or “peptide portion” is used broadly herein to mean two or more amino acids linked by a peptide bond. The term “fragment” is used herein to refer to a portion of a full-length polypeptide or protein, such portion which can be produced by a proteolytic reaction on a polypeptide, i.e., a peptide produced upon cleavage of a peptide bond in the polypeptide. It should be recognized that the fragment need not necessarily be produced by a proteolytic reaction but can be produced using methods of chemical synthesis or methods of recombinant DNA technology, to produce a synthetic polypeptide. It should be recognized that the term “polypeptide” is not used herein to suggest a particular size or number of amino acids comprising the molecule and that a peptide of the invention can contain up to several amino acid residues or more.

By “isolated” or “purified” is meant a composition (e.g., a polypeptide or nucleic acid) that is substantially free from other materials, including materials with which the composition is normally associated in nature. The polypeptides of the invention, or fragments thereof, can be obtained, for example, by extraction from a natural source (e.g., phage), by expression of a recombinant nucleic acid encoding the polypeptide (e.g., in a cell or in a cell-free translation system), or by chemically synthesizing the polypeptide. In addition, polypeptide fragments may be obtained by any of these methods, or by cleaving full length polypeptides. A fragment of a reference protein or polypeptide includes only contiguous amino acids of the reference protein/polypeptide, and is at least one amino acid shorter than the reference sequence.

Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule but deletion can range from 1-30 residues. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, for example, M13 primer mutagenesis and PCR mutagenesis. Amino acid substitutions are typically of single residues, but can occur at a number of different locations at once; insertions usually will be on the order of about from 1 to 10 amino acid residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure unless such a change in secondary structure of the mRNA is desired. Substitutional variants are those in which at least one residue has been removed and a different residue inserted in its place. Such substitutions generally are made in accordance with the following Tables 1 are referred to as conservative substitutions.

TABLE 1
Amino Acid Substitutions

| Original | Exemplary |
|---|---|
| Residue | Substitutions |
| Ala | Ser |
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Pro | Gly |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example, as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, and (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations shown in Table 1. Conservatively substituted variations of each explicitly disclosed sequence are included within the polypeptides provided herein.

A substitution of an amino acid residue for another amino acid residue having similar biochemical properties. Typically, conservative substitutions have little to no impact on the biological activity of a resulting polypeptide. In a particular example, a conservative substitution is an amino acid substitution in a peptide that does not substantially affect the biological function of the peptide. A peptide can include one or more amino acid substitutions, for example 2-10 conservative substitutions, 2-5 conservative substitutions, 4-9 conservative substitutions, such as 2, 5 or 10 conservative substitutions.

A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using standard peptide synthesis methods. An alanine scan can be used to identify which amino acid residues in a protein can tolerate an amino acid substitution. In one example, the biological activity of the protein is not decreased by more than 25%, for example not more than 20%, for example not more than 10%, when an alanine, or other conservative amino acid (such as those listed below), is substituted for one or more native amino acids.

Further information about conservative substitutions can be found in, among other locations, in Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. For example, there are numerous D amino acids or amino acids which have a different functional substituent than the amino acids shown in Table 1. The opposite stereoisomers of naturally occurring peptides are disclosed, as well as the stereoisomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14 (10):400-403 (1989); Benner, TIB Tech, 12:158-163 (1994); Ibba and Hennecke, Bio/technology, 12:678-682 (1994), all of which are herein incorporated by reference at least for material related to amino acid analogs).

Molecules can be produced that resemble polypeptides, but which are not connected via a natural peptide linkage. For example, linkages for amino acids or amino acid analogs can include CH2NH—, —CH2S—, —CH2-CH2-, —CH═CH—(cis and trans), —COCH2-, —CH(OH)CH2-, and —CHH2SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH2NH—, CH2CH2-); Spatola et al. Life Sci 38:1243-1249 (1986) (—CHH2-S); Hann J. Chem. Soc Perkin Trans. 1307-314 (1982) (—CH—CH—, cis and trans); Almquist et al. J. Med. Chem. 23:1392-1398 (1980) (—COCH2-); Jennings-White et al. Tetrahedron Lett 23:2533 (1982) (—COCH2-); Szelke et al. European Appln, EP 45665 CA (1982): 97:39405 (1982) (—CH(OH)CH2-); Holladay et al. Tetrahedron. Lett 24:4401-4404 (1983) (—C(OH)CH2-); and Hruby Life Sci 31:189-199 (1982) (—CH2-S—); each of which is incorporated herein by reference. A particularly preferred non-peptide linkage is —CH2NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

The term “antibodies” is used herein in a broad sense and includes both polyclonal and monoclonal antibodies. In addition to intact immunoglobulin molecules, also included in the term “antibodies” are human or humanized versions of immunoglobulin molecules or fragments thereof, as long as they are chosen for their ability to interact with endocytic receptors such as DEC205, DC-SIGN, Langerin, or CD64/FcγR1. The antibodies can be tested for their desired activity using the in vitro assays described herein, or by analogous methods, after which their in vivo therapeutic and/or prophylactic activities are tested according to known clinical testing methods.

The term “antibody” as used in this invention is meant to include intact molecules as well as fragments thereof, such as, for example, Fab, and F(ab′)₂, which are capable of binding the epitopic determinant.

As used herein, the term “antibody or fragments thereof” encompasses chimeric antibodies and hybrid antibodies, with dual or multiple antigen or epitope specificities, and fragments, such as F(ab′)₂, Fab′, Fab and the like, including hybrid fragments. Thus, fragments of the antibodies that retain the ability to bind their specific antigens are provided. For example, fragments of antibodies which maintain endocytic receptor-binding activity are included within the meaning of the term “antibody or fragment thereof.” Such antibodies and fragments can be made by techniques known in the art and can be screened for specificity and activity according to the methods set forth herein and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies; scFv) as described, for example, in U.S. Pat. No. 4,704,692, the contents of which are hereby incorporated by reference.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, when attached to other sequences can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147 (1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Disclosed herein are nucleic acids that encode a surface polypeptide of the invention. The surface polypeptide can be gpD. For example, the nucleic acid encoding gpD can comprise sequences of SEQ ID NO: 1 or 52 in the presence or absence of modifications. The nucleic acid encoding gpV can comprise sequences of, for example, SEQ ID NO:53 in the presence or absence of modifications. Provided herein are nucleic acids that encode modified surface polypeptides that enhance targeting to antigen presenting cells. Further provided is a nucleic acid that encodes a surface polypeptide fusion protein, wherein the fusion protein destabilizes a viral capsid. The encoded polypeptide fusion protein can comprise a PEST sequence. The PEST sequence can be a PEST sequence from LLO or a related listerial lysin. The nucleic acid encoding the PEST sequence can comprise the nucleic acid sequences encoding the amino acid sequences of SEQ ID NO:16, 17, 18, or 19. All of the provided nucleic acids can further comprise a nucleotide sequence that encodes a basic peptide transduction domain. The basic PTDs can be derived from Antennipedia, HIV-1 Tat, or Pep1. The nucleic acid encoding the basic PTDs can comprise nucleic acid sequences encoding the amino acid sequences of SEQ ID NO:25, 26 or 27. Also provided herein are nucleic acid sequences that encode the modified surface polypeptides, wherein the modified surface polypeptides comprise one or more modification of the invention. Further provided are phages comprising the nucleic acids that encode the modified surface polypeptides of the invention.

Disclosed are nucleic acid molecules comprising a sequence encoding a peptide having at least 80%, 90%, or 95% identity to a peptide set forth in SEQ ID NOs:2-5, 7, 9-13, 15-28, 30, 34-41, or 43-49.

The nucleic acids encoding the modified surface polypeptide can include nucleotide analogues (i.e., modified nucleotides), and lipid moieties (e.g., a cholesterol moiety).

A variety of sequences are provided herein and these and others can be found in Genbank, at http://www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

Provided herein is a vector comprising any of the nucleic acids provided herein, operably linked to an expression control sequence. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter or EF1 promoter, or from hybrid or chimeric promoters (e.g., cytomegalovirus promoter fused to the beta actin promoter). The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

“Enhancer” generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone, synthetic transcription factors, directed RNA self-cleavage (Yen L. et al. 2004. Nature 431:471-476), and other approaches. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (plus a linked intron sequence), beta-actin, elongation factor-1 (EF-1) and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial (astrocytic) origin. The HLA-DR, CD11c, Fascin and CD68 promoters have all been used to selectively express genes in antigen-presenting cells, including macrophages and dendritic cells (Brocker, T., et al. 1997. J Exp Med 185:541-550; Gough P. J. and Raines, E. W. 2003. Blood 101:485-491; Cui, Y. et al. 2002. Blood 99:399-408; Sudowe, S. et al. 2003. Mol Ther 8:567-575), and promoter elements from dendritic cell-specific genes (such as CD83) may also prove useful in this regard (Berchtold S. et al. 2002. Immunobiology 205:231-246).

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

The viral vectors can include nucleic acid sequence encoding a marker product. This marker product is used to determine if the gene has been delivered to the cell and once delivered is being expressed. Preferred marker genes include the E. coli lacZ gene, which encodes β-galactosidase, green fluorescent protein (GFP), and luciferase.

In some embodiments the marker may be a selectable marker. Examples of suitable selectable markers for mammalian cells are dihydrofolate reductase (DHFR), thymidine kinase, neomycin, neomycin analog G418, hygromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

Provided herein is a method of promoting an antigenic response in a subject, comprising administering to the subject the delivery system provided herein. The method can further comprise the co-administration of the provided modified phage with agents intended to increase the efficiency of phage uptake into antigen-presenting cells. Thus, the method can further comprise administering to the subject a granulocyte colony stimulating factor (G-CSF) or a variant or fragment thereof. Engagement of the G-CSF receptor results in the enhanced phagocytosis of IgG-coated particles (Santini, V., et al. 2003. Blood 101:4615-22). Provided herein is the use of G-CSF to increase uptake of opsinized or antibody-coated phage particles. It can be delivered as recombinant protein, formulated with the phage particles, or delivered in the form of a DNA plasmid encoding G-CSF, before, after or simultaneous with phage delivery.

The method can further comprise administering to the subject a Toll-like receptor ligand. CpG oligonucleotides that interact with Toll-like receptor-9 (TLR9) increase phagocytosis of bacteria by macrophages (Utaisincharoen, P., et al. 2003. Clin Exp Immunol 132:70-5), and engagement of other TLRs increases phagocytosis by dendritic cells (West, M. A., et al. 2004. Science 305:1153-7). Thus, provided herein is the use of CpG oligonucleotides and other small molecule ligands of mammalian TLRs to enhance the intracellular uptake of the provided phage vectors. Several CpG oligonucleotides and TLR ligands have already been developed for human use and/or tested in human clinical trials, including CpG 7909 (SEQ ID NO:32), resiquimod (R-848) and related molecules (developed by 3M) and other similar materials (Cooper, C. L., et al. 2004. Vaccine 22:3136-43; Lore, K., et al. 2003. J Immunol 171:4320-8; Sauder, D. N., et al. 2003. Antimicrob Agents Chemother 47:3846-52). Note that CpG7909 can be synthesized with a wholly phosphorothioate backbone. In this form, it can be used safely in humans (Cooper, C. L., et al. 2004. Vaccine 22:3136-43). These molecules have adjuvant activity due, in part, to their effects on differentiation of dendritic cells. However, provided herein is a different use for these compounds—the selective enhancement of phage uptake into antigen-presenting cells. In the provided methods, these molecules are optimally formulated with the phage particles or administered before, after or simultaneous with the modified phage.

The method can further comprise administering to the subject a PTD-derived polypeptide. PTD-containing peptides and other carrier peptides can be coadministered with macromolecules such as recombinant proteins or mammalian virus vectors, for purposes of enhancing gene/protein transfer into mammalian cells (Gratton, J. P., et al. 2003. Nat Med 9:357-62; Morris, M. C., et al. 2001. Nat Biotechnol 19:1173-6; Wadia, J. S., et al. 2004. Nat Med 10:310-5). Provided herein is the use of this approach to enhance the escape of the provided phage from membrane-delineated intracellular compartments and entry into cytosol.

The provided phage vectors can be used in heterologous prime-boost vaccination regimens, thereby reducing the cost of immunization (due to the low cost of phage vectors) while also increasing its effectiveness (by eliciting improved immune responses) (Amara, R. R., et al. 2001. Science 292:69-74, Degano, P., et al. 1999. Vaccine 18:623-32; M., et al. 1998. Eur J Immunol 28:4345-55; Schneider, J., et al. 1999. Immunol Rev 170:29-38). It is understood that one can effectively utilize various combinations of phage vectors and mammalian viral vectors, as well as phage plus recombinant protein, and phage plus DNA plasmid vectors, in successful immunization regimens. Such regimens can also include synthetic or natural adjuvants, including molecularly designed approaches (cytokines, cytokine-encoding plasmids/vectors), Toll-like receptor (TLR) ligands, and the use of chemokines and other molecules intended to recruit and activate dendritic cells (Barouch, D. H., et al. 2000. Science 290:486-92; Chen, K., et al. 1997. Cancer Research 57:3511-6; Lore, K., et al. 2003. J Immunol 171:4320-8; Moore, A. C., et al. 2002. J Virol 76:243-50). Therefore, the provided method can further comprise administering to the subject a viral vector comprising the antigen. Optimally, the lambda phage is administered prior to the viral vector.

It is also possible to insert multiple modifying peptides within a single surface polypeptide. For example, the molecular structure of gpD shows it to be highly flexible, in terms of its ability to accept extended exogenous peptide sequences at both its N and C terminus (Yang, F., et al. 2000. Nat Struct Biol 7:230-7). This approach can be further enhanced through the use of flexible linker peptides.

Thus, provided herein is a method of making a lambda phage with a modified surface polypeptide, comprising the steps of a) inserting into a surface polypeptide-encoding nucleic acid more than one nucleotide sequence that encodes exogeneous polypeptide sequences; and b) expressing the surface polypeptide and exogenous polypeptide sequences in the lambda phage. The exogeneous polypeptide sequences can be encoded at both the N and C terminus of the modified surface polypeptide. The exogeneous polypeptide sequences can include flexible linker polypeptides. These can include, for example, alternating Serine and Glycine stretches exemplified by (GGGGS)₃ (SEQ ID NO: 54) (Freund C. et al. 1993. FEBS Lett 320:97-100), (GSGSGS)ₙ (SEQ ID NO: 55), and G(SGGG)₂SGGT (SEQ ID NO:57); the flexible linker peptide of Trichoderma reesi cellobiohydrolase I (CBHI) (Takkinen, K. et al. 1991. Prot Eng 4:837-841); and elbow-like peptides such as SAKTTP (SEQ ID NO:50), RADAAP (SEQ ID NO:51), and derivatives thereof (see Le Gall F., et al. 2004. Protein Eng Des Sel 17:357-366, which is incorporated herein by reference in its entirety for all derivatives and methods of making same). A single modified surface polypeptide can include 1, 2, 3, 4, 5, 6, 7, 8, 9, or 10 of the provided polypeptide motifs. It is understood that any of the provided modifications can be used in this combination. For example, provided is the combination of a PTD encoding nucleic acid with any of the other provided surface polypeptide modifications.

Further provided is the use of an in vivo trans-complementation approach to generate phage particles which display >1 different surface polypeptides, e.g. gpD, on their surface. This can be achieved for gpD, for example, by transforming a gpD-deficient lambda lysogen with several plasmid DNA constructs (each containing a unique combination of selectable antibiotic-resistance markers and compatible origin sequences) that encode the various desired gpD fusion proteins or polypeptide sequences. Alternatively, a gpD-deficient lysogen can be transformed with a single plasmid construct that simultaneously expresses several gpD fusion proteins (from different promoter elements). Generation of these constructs can be facilitated by the use of synthetic gene design, using different codons to encode the same amino-acid sequences from non-homologous sequences. This reduces the extent of genetic homology between the various gpD expression cassettes, thereby reducing their propensity to undergo homologous recombination.

Thus, provided herein is a method of making a lambda phage with a plurality of modified surface polypeptides, comprising the steps of a) transforming a surface polypeptide-deficient lambda lysogen with a plurality of nucleic acids, wherein the nucleic acids encode a plurality of modified surface polypeptides, wherein the surface polypeptides comprise exogenous polypeptides; and b) expressing the surface polypeptide and exogenous polypeptide sequences in the lambda phage.

Also provided is a method of making a lambda phage with a modified surface polypeptide, comprising the steps of (a) transforming a surface polypeptide-deficient lambda lysogen with a plurality of nucleic acids, wherein the nucleic acids encode a plurality of gpD proteins, wherein at least one nucleic acid encodes a modified gpD protein and wherein the surface polypeptides comprise exogenous polypeptides; and (b) expressing the surface polypeptide and exogenous polypeptide sequences in the lambda phage.

The transformation step can be performed using a plasmid construct encoding a plurality of modified surface polypeptides. A single plasmid can comprise 1, 2, 3, 4, 5, 6, 7, 8, 9, or 10 unique modified surface polypeptides. It is understood that any of the provided modifications can be used in this combination. For example, provided is a plasmid comprising the combination of a PTD encoding nucleic acid with any of the other provided surface polypeptide modifications.

The transformation step of the method can be performed using a plurality of plasmids that each encode one or more modified surface polypeptides. A single phage can comprise 1, 2, 3, 4, 5, 6, 7, 8, 9, or 10 unique plasmids that encode modified surface polypeptides. It is understood that any of the provided modifications can be used in this combination. For example, provided is a phage comprising the combination of a PTD encoding nucleic acid with any of the other provided surface polypeptide modifications.

The provided delivery system can be used to deliver antigens to a subject to target antigen presenting cells for the purpose of eliciting an antigenic response to treat infectious or acquired diseases. These diseases can include viral infections (e.g., influenza, HIV/AIDS) bacterial infections (e.g., bacterial meningitis) parasitic infections (e.g., fungal infections) neurological diseases (e.g., Alzheimer's disease), cancer, etc. The antigen of the provided invention could be any known or discovered nucleic acid or polypeptide sequence that can elicit an antigenic response. Examples of known antigens that can be used with the provided compositions and methods include Her2/Neu (a breast cancer antigen), “conformational” antigens such as the Aβ protein or prion-disease proteins (both of which represent mis-folded or aggregated derivatives of normal cellular proteins) as well as surface, structural, enzymatic or regulatory proteins encoded by microbial pathogens. Examples of such antigens include: HIV-1 Env, Gag, Pol, Tat, Rev, Vif, Vpr and Nef, as well HCMV gB and gH/gL, influenzavirus HA and NA proteins, M. tuberculosis proteins ESAT6 and HSP65, malarial proteins such as thrombospondin-related adhesion protein (TRAP), and many more. Examples of viral pathogens that might be subject to control by vaccines include HIV-1, hepatitis B and C viruses, cytomegalovirus, Dengue virus, respiratory syncytial virus, the SARS coronavirus, Ebolavirus, Lassa, influenzavirus, and many more. Examples of bacterial pathogens and antigens of interest include bacterially-encoded toxins and toxin-related molecules (such as the anthrax edema factor (EF), lethal factor (LF) and protective agent (PA) as well as botulinum toxin, and virulence factors and toxins encoded by Fransicella tularensis, Yersinia pestis and other biodefense agents. Other known antigens may include surface, structural, regulatory or enzymatic proteins from malarial parasites and Mycobacterium tuberculosis, or from other medically important pathogens and “dual use” agents with potential for application in biowarfare. Preferably, the antigenic response will be sufficient to induce a protective humoral or cellular immune response in the subject.

The compositions can be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, intradermally, transdermally, extracorporeally, topically or the like, including topical intranasal administration or administration by inhalant. For example, provided is a method of eliciting an immune response in a subject, comprising intradermally administering to the subject a modified lambda phage provided herein. It has also been shown that lambda is capable of withstanding the harsh conditions encountered during oral administration (Jepson, C. D. and J. B. March. 2004. Vaccine 22:2413-9). Orally administered phage have been reported to reach the bloodstream for multiple species of bacteriophage (Hildebrand, G. J. and H. Wolochow. 1962. Proc Soc Exp Biol Med 109:183-5; Reynaud, A., et al. 1992. Vet Microbiol 30:203-12; Weber-Dabrowska, B., et al. 1987. Arch Immunol Ther Exp 35:563-8). Furthermore, the specific targeting peptide sequences that allow phage to pass through the intestinal wall and thereby enter the general circulation can be used (Duerr, D. M., et al. 2004. J Virol Methods 116:177-80).

As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA and Cell Biology 10:6, 399-409 (1991)).

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

Following administration of a disclosed composition, such as an antigen delivery vehicle, the efficacy of the antigen delivery vehicle can be assessed in various ways well known to the skilled practitioner. For instance, one of ordinary skill in the art will understand that a composition, such as an antigen delivery vehicle, disclosed herein is efficacious in eliciting an antigenic response in a subject by observing a humoral response. Optimally, a protective immune response is shown by observing that the composition prevents a further infection.

The compositions disclosed herein and the compositions necessary to perform the disclosed methods can be made using any method known to those of skill in the art for that particular reagent or compound unless otherwise specifically noted.

For example, the nucleic acids can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

One method of producing the disclosed polypeptides is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY, which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Derivation of Surface-Modified Lambda Recombinants Intended for Targeted Gene Delivery to Antigen-Presenting Cells

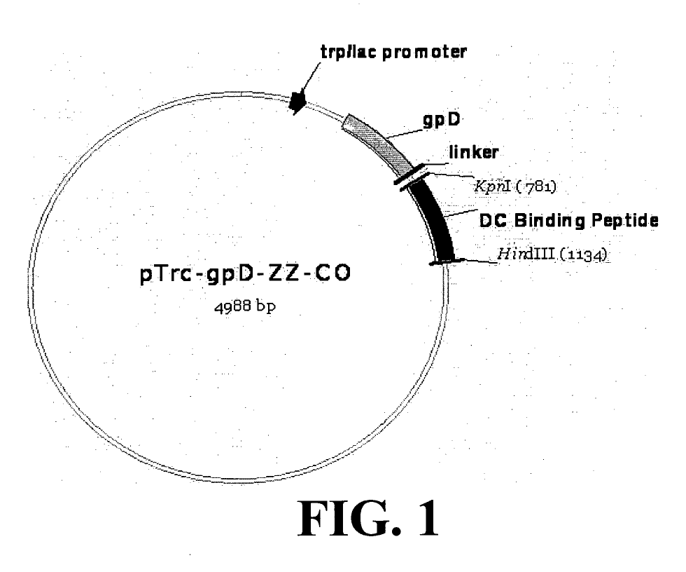

An E. coli expression plasmid was generated for the production of gpD fusion proteins (FIG. 1). The histidine tag was removed from pTrcHisA plasmid (Invitrogen, Carlsbad, Calif.) and an E. coli codon optimized gpD sequence (SEQ ID NO:1) was added after the trp/lac promoter. A 13 amino acid linker sequence joined gpD and the targeting peptide sequence. All targeting peptides were cloned between the unique KpnI and HindIII sites.

The synthetic, E. coli codon optimized gpD gene sequence increases E. coli expression of the peptide fusions. In addition, the codon optimized gpD has about 76% homology with the original gene, decreasing the potential for lambda to recombine and replace its nonfunctional gpD with the supplemented plasmid. The targeting peptides were fused to the carboxy terminus of gpD. The carboxy terminus has been demonstrated by X-ray crystallography to be more accessible for peptide display (Yang, F., et al. 2000. Nat Struct Biol 7:230-7). Finally, a short, 13 amino acid glycine and serine linker sequence was added. According to the crystal structure, both gpD termini point towards the lambda capsid (Yang, F., et al. 2000. Nat Struct Biol 7:230-7). A linker sequence increases surface exposure of the targeting peptide. Constructed plasmids were transformed into E. coli strain Top 10, verified by sequencing, and checked for IPTG inducible protein expression by immunoblot.

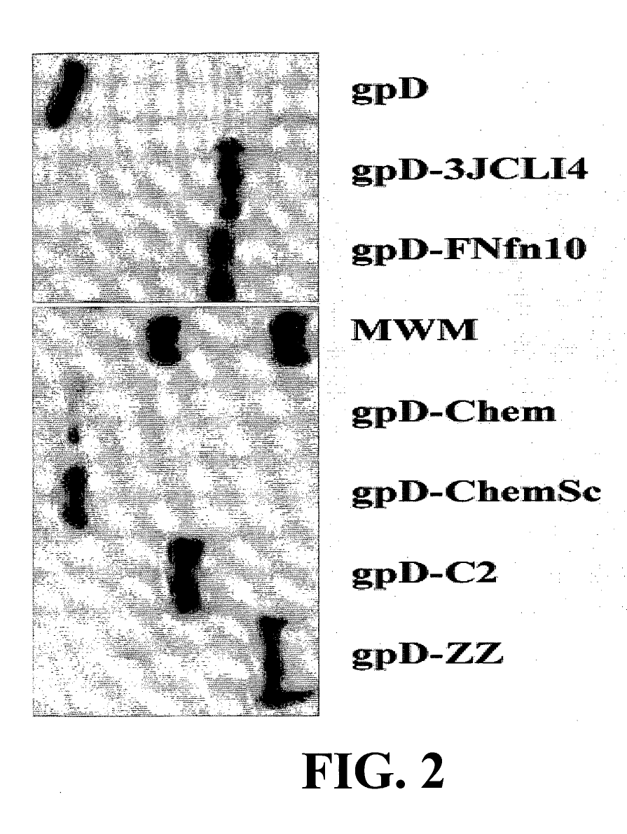

The indicated sequences (3JCLI4, Fnfn10, Chemerin peptide, scrambled chemerin peptide, C2 and ZZ domains) were inserted at the carboxy terminus of gpD (see FIG. 1). E. coli transformed with these plasmids were then propagated until early log phase and lyzed. Cellular lysates were separated by SDS-PAGE and gpD fusions were identified by immunoblot with an anti-gpD antibody (FIG. 2). Fusions of the expected molecular weight, as compared to protein ladder (MagicMark Ladder; Invitrogen, Carlsbad, Calif.) were visualized for all plasmids.

The in trans complementation system first described by Sternberg and Hoess and adapted by Eguchi et al. allowed for the display of gpD-peptide fusions on lambda (Eguchi, A., et al. 2001. J Biol Chem 276:26204-10; Sternberg, N., and R. H. Hoess. 1995. Proc Natl Acad Sci USA 92:1609-13). Lysogens for gpD deficient lambda were maintained by a temperature sensitive mutation in one of the proteins necessary for repression of the lytic lifecycle. Lysogens were transformed with the desired gpD-peptide expression plasmid and phage excision was promoted by altering temperature conditions during growth of the lysogen culture. Lambda then incorporate the gpD-peptide fusions into the coat heads during cytoplasmic assembly.

E. coli lysogenic for recombinant lambda phage vectors were transformed with the desired gpD-peptide expression plasmid. Lysogens were grown to log phase and then lambda excision induced by increasing the culture temperature to 42° C. Recombinant lambda were lysed from the E. coli host and purified by standard cesium chloride equilibrium gradient. Purified phage were titered on LE392 E. coli and examined for appropriate gpD fusion by SDS-PAGE and immunoblot. Calculated infectious titer can be compared to total particles present by protein and DNA concentration gels.

The purification of lambda was necessary to remove potentially toxic bacterial endotoxins. Although endotoxin levels were evaluated by one of many commercially available kits, cesium chloride purification is reported to decrease endotoxin content in phage preps by more than 100 fold with a final reported level of 3 EU/ml for lambda (Merril, C. R., B et al. 1996. Proc Natl Acad Sci USA 93:3188-92). Additional chromatographic methods have been described for removal of LPS from lambda phage preparations (Boratynski, J., et al. 2004. Cell Mol Biol Lett 9:253-9), as has the use of the detergent Triton X-114 (Cotten, M., et al. 1994. Gene Ther 1:239-46).

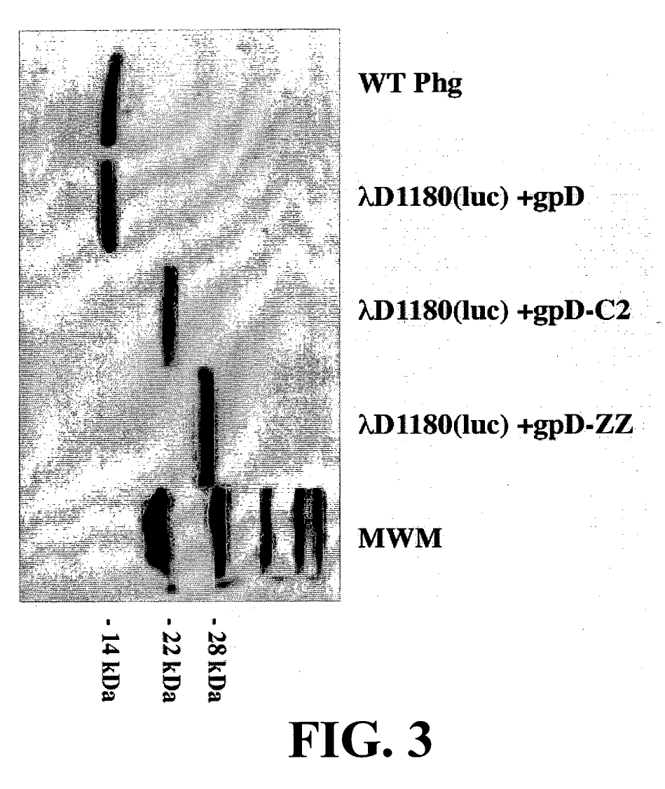

The gpD-encoding plasmids (gpD, gpD-C2, gpD-ZZ) were transformed into D-deficient lambda phage λ D1180(luc) lysogens and recombinant phage purified by cesium chloride equilibrium gradient. Phage were titered on E. coli strain LE392, and 1×10⁸ pfu of each phage were then lysed, separated by SDS/PAGE, and visualized on immunoblot with a polyclonal rabbit antiserum against gpD (FIG. 3). All of the gpD fusion proteins were of the expected size, as compared to a protein standard (MagicMark Ladder; Invitrogen, Carlsbad, Calif.).

Lambda expression was induced by growing lysogens in liquid media and ampicillin (100 μg/ml) at 32° C. with vigorous aeration until an OD₆₀₀ of 0.5. Cultures were than incubated with gentle rotation in a 45° C. water bath for 20 minutes after the culture reached an internal temperature of 42° C. The cultures were immediately cooled to 37° C. in an ice bath and shaken for an additional 3 hours at 38° C. Bacteria was concentrated by centrifugation and lysed with chloroform and DNase to release entrapped phage particles. Bacterial debris was cleared by centrifugation and the resulting supernatant was centrifuged at 25,000 rpm for 60 minutes to pellet phage. Pelleted lambda was resuspended in phage suspension media and purified by cesium chloride equilibrium gradient. Cesium chloride purified phage were dialyzed into magnesium supplemented Tris buffer and stored at 4° C. with 10 μl of chloroform.

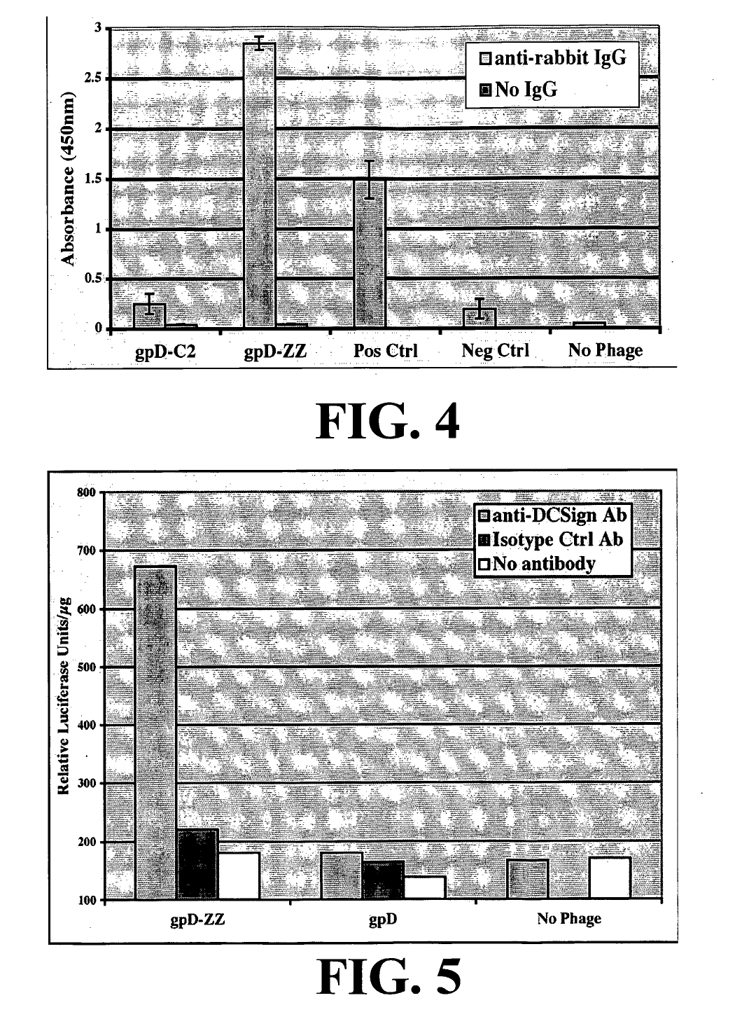

Ninety-six well plates were coated with gpD-C2 or gpD-ZZ phage. Bound phage was then incubated with an anti-rabbit IgG-HRP and bound antibodies were detected by spectrophotometry. gpD phage incubated with anti-gpD rabbit antibody and anti-rabbit IgG-HRP antibody served as the positive control (FIG. 4). Negative controls included gpD-ZZ and gpD-C2 phage with no antibody, gpD phage with just anti-rabbit IgG-HRP, and antibody without phage.

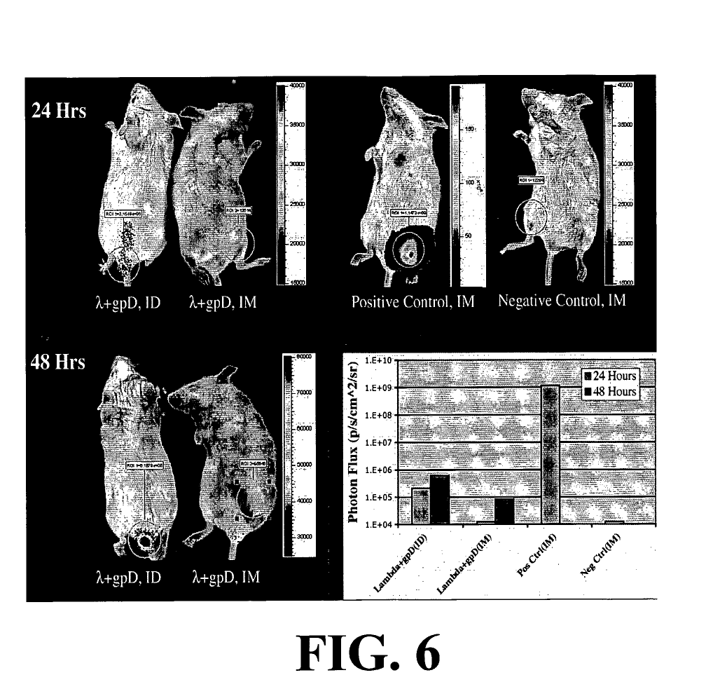

Phage (1×10¹⁰ pfu) were pre-complexed with antibodies to DC-SIGN (2 μg/ml) for 1 hour at 37° C. The antibody-phage conjugates were then added to DC-SIGN expressing HeLa cells, for 2 hours at 37° C. and an MOI of 1×10⁵. All infections were performed in the presence of 100 μM chloroquine, and included spinoculation-mediated enhancement of infection (centrifugation at 2000 rpm, at 30° C. for 40 minutes). Luciferase expression was measured at 48 hours post infection (FIG. 5), indicating enhanced transduction of DC-SIGN positive human cells using lambda phage bearing gpD-ZZ.

### Example 2

### GpD-Deficient Lambda Genomes Containing a Mammalian Expression Cassette can be Successfully Complemented with Wild-Type GpD Using a Plasmid Expression System, and the Resulting Phage Particles are Capable of Successfully Transducing Live Animals. [Dr. Dewhurst: this Section was Prophetic. Is this Redundant with New Data? can You Merge them?]

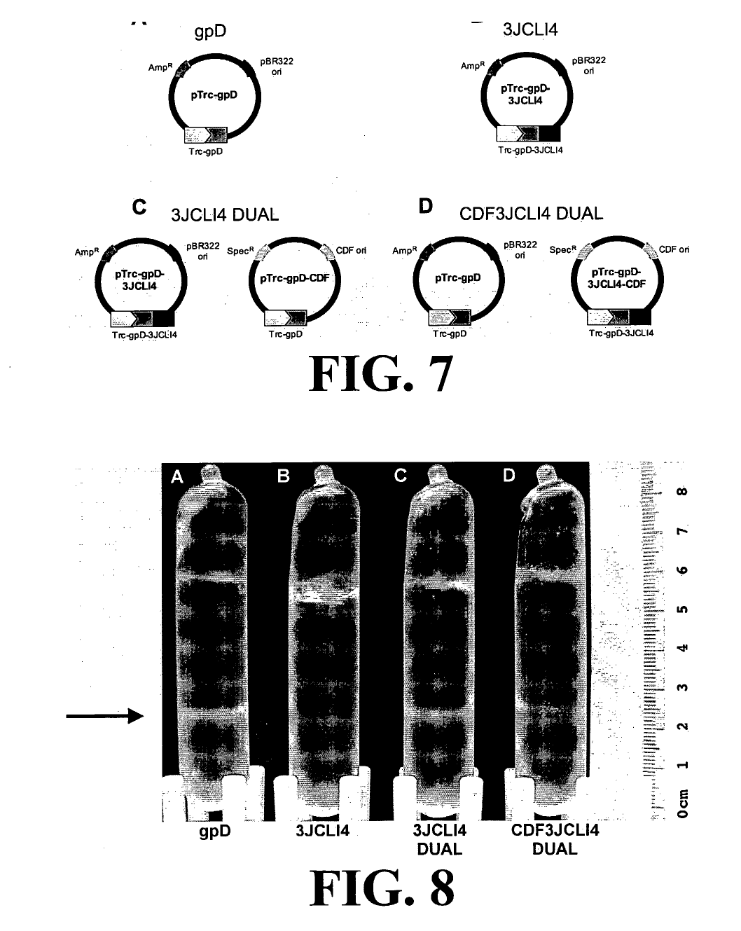

λ1180(luc) was propagated in E. coli cells that expressed wild-type gpD. The resulting phage (λ+gpD) was CsCl-gradient purified and injected intramuscularly (IM) or intradermally (ID) at 1×10¹¹ pfu or ID at 5×10¹⁰ pfu in 5-6 week old Balb/c mice. Controls were injected IM with 1×10⁹ pfu of a luciferase-expressing adenovirus (positive) and 1×10¹⁰ pfu of wild type lambda (negative). For imaging of luciferase expression, a luminescent substrate (Luciferin, 4.3 μg) was injected IP at 24 and 48 hours and mice were imaged using a Xenogen (Alameda, Calif.) IVIS imaging system at 10 minutes post injection (FIG. 6), demonstrating that D-deficient lambda phage complemented with a wild-type gpD expression plasmid successfully mediated gene transfer in vivo.

### Example 3

### Immunization of Mice with Recombinant Lambda Phage that Express the Surface Glycoprotein from HIV-1 (gp120) and which are Targeted to Antigen-Presenting Cells Through Specific Modifications of GpD

Genomic DNA of λD1180 can be ligated at the Cos I sites, digested at the unique EcoRI site, and treated with calf intestinal alkaline phosphatase. A mammalian expression cassette, encoding HIV-1 gp120 under the transcriptional control of a strong, constitutive promoter element such as the CMV immediate-early promoter or the CMV/β-actin (CAG) hybrid CAG promoter, can be amplified by PCR and ligated to the λD1180 DNA. The ligation can be packaged using a Lambda Packaging Kit (Amersham, Piscataway, N.J.) and clones can be screened by plaque PCR. Successful gp120 insertion can be verified by gene sequencing and the appropriate clone can be amplified by plate lysate. Top10 E. coli (Invitrogen, Carlsbad, Calif.) can be infected with the purified λD1180 (gp120) clone at an MOI of 1×10⁶. The infection an be plated and incubated overnight at 32° C. Bacterial colonies can be screened by replicate plating for temperature sensitivity at 38° C. PCR can be performed on temperature sensitive colonies in order to identify single integration lambda lysogens.

λD1180 (gp120) lysogens can be transformed with the desired gpD fusion plasmid and the phage can be induced and purified according to the above mentioned protocols. Lambda phage particles can be collected and subjected to SDS-PAGE analysis, in order to confirm the presence of the expected protein fusions; phage preparations can also be analyzed for EDTA sensitivity (to ensure that gpD complementation is effective). Purified, endotoxin-free phage preparations can be generated by CsCl density gradient centrifugation (1-3 times), followed by Triton X-114 extraction and/or purification over a polymyxin B column (Associates of Cape Cod). Phage can then be administered alone or in conjunction with various adjuvants by intradermal and intramuscular injection into BALB/c mice. A single inoculation of phage, or several inoculations of phage can be tested—both alone and in combination with a standard saline DNA plasmid, or an adenovirus vector, encoding the same gp120 antigen. A variety of different prime-boost regimens can be explored, including the following:

TABLE 2
Inoculation Regimens for Phage Immunogenicity Studies

| Prime | Boost | Boost | Boost |
|---|---|---|---|
| (day 0) | (3 weeks) | (7 weeks) | (11-15 wks) |
| Phage | | | |
| Phage | Phage | | |
| Phage | Phage | Phage | |
| Phage | Phage | Phage | Phage |
| Phage | DNA | | |
| DNA | Phage | | |
| Phage | Adenovirus | | |
| Adenovirus | Phage | | |

This list represents a partial list of possible prime-boost strategies; all inoculations can be performed by at least two different routes (intramuscular, intradermal tailbase), in the presence or absence of desired adjuvants (e.g., CpG oligonucleotides, endolysosomal-targeted peptides, etc).

Mice can be sacrificed 14 days following the final vector injection, and cellular immune responses to gp120 can be analyzed by stimulation of splenocytes with overlapping gp120 peptide pools, followed by intracellular cytokine staining of CD4+ and CD8+ cell populations for, respectively, IL-2, and IFN-γ. Tetramer staining can also be performed using a H-2D(d) restricted tetrameric complex loaded with the highly immunodominant HIV-1 V3 peptide, RGPGRAFVTI (SEQ ID NO:66). Finally, serologic responses to gp120 can be analyzed by IgG serum ELISA, using highly purified gp160 target antigen (Protein Sciences).

### Example 4

### A Method for Displaying Recalcitrant Proteins on the Surface of Bacteriophage Lambda

Lambda D1180 (Luc) genomes contain a mammalian expression cassette that encodes for firefly luciferase under the transcriptional control of the human cytomegalovirus immediate early promoter. An E. coli codon-optimized (ECO) derivative of the wild-type λ gpD gene was generated synthetically (GeneArt, Regensburg, Germany) and inserted into the pTrcHis plasmid (Invitrogen, Carlsbad, Calif.). The optimized sequence was selected on the basis of Entelechon's proprietary gene design software (http://www.entelechon.com/), which performs codon optimization for the species of interest, while avoiding specified restriction enzyme sites. The corresponding sequence is available via GenBank, with the accession number DQ156943. The resulting construct, pTrc-gpD-fusion contained the codon-optimized gpD sequence (with its authentic ATG codon, but without a translational stop codon), followed by a short flexible linker sequence [G(SGGG)₂SGGT, SEQ ID NO:57] and then by BamHI and KpnI restriction sites; desired fusion partners of interest were inserted between the KpnI site and a terminal HindIII site. Note that, during the process of cloning, the native, promoter-proximal NcoI site within pTrc was eliminated. Other linker sequences that can be used include, but are not limited to, alternating Serine and Glycine stretches exemplified by (GGGGS)₃ (SEQ ID NO: 54) (Freund C. et al. 1993. FEBS Lett 320:97-100), (GSGSGS)ₙ (SEQ ID NO: 55), and G(SGGG)₂SGGT (SEQ ID NO:X); the flexible linker peptide of Trichoderma reesi cellobiohydrolase I (CBHI) (Takkinen, K. et al. 1991. Prot Eng 4:837-841); and elbow-like peptides such as SAKTTP (SEQ ID NO:50), RADAAP (SEQ ID NO:51), and derivatives thereof (Le Gall F., et al. 2004. Protein Eng Des Sel 17:357-366).

In order to derive a construct that expressed gpD alone, the gpD insert was subjected to polymerase chain reaction (PCR) amplification using primers that added a translational stop codon at the 3′ end of the gpD gene. The oligonucleotide primers used for this amplification were: (i) gpD-StopFOR (5′-aagctttcATGaccagcaag-3′, SEQ ID NO:58) and (ii) gpD-StopREV (5′-atctaagcttCTAtacaatactgattgcggtg-3′, SEQ ID NO:59); underlined sequences denote the added BspHI and HindIII sites, respectively, while capitalized boldface letters denote the ATG codon and inserted translational stop codon, respectively. The resulting PCR product was restricted with BspHI and HindIII and then inserted into the parental pTrc vector using the NcoI and HindIII restriction sites, to create pTrc-gpD.

A series of plasmid constructs were created in which specific sequences of interest were fused to gpD. The exogenous DNA sequences chosen included an insert encoding for a high-affinity αᵥβ₃ binding protein derived from the tenth fibronectin type II domain (3JCL14) (Richards, J., et al. (2003)). This was PCR amplified from an available parental plasmid (Richards, J., et al. (2003)), using the following primers: 3JCLI4FOR (5′-atcggtacccaggtttctgatgttccgcgt-3′, SEQ ID NO:60) and 3JCLI4REV (5′-ggccaagcttCTAggtacggtagttaatcg-3′, SEQ ID NO:61). The PCR product was then digested with KpnI and HindIII (these sites are underlined in the primer sequences) and inserted into the corresponding restriction sites of our pTrc-gpD-fusion vector, so as to create pTrc-gpD-3JCLI4.

In order to create plasmid vectors that permit expression of two different forms of gpD within the same E. coli host cell, the pBR322-derived pMB1 origin of DNA replication in the pTrc-based expression plasmids (approximately 20 copies/chromosome equivalent; Bolivar, F., et al. (1977); Brosius, J., et al. (1982)) was replaced with a compatible origin of DNA replication derived from the CloDF13 replicon (approximately 20-40 copies/chromosome equivalent; Nijkamp, H. J., et al. (1986); Kim, J. S, and Raines, R. T. (1993)). The CDF-derived origin and flanking antibiotic resistance marker were amplified by PCR from the pCDF-1b plasmid (Novagen, San Diego, Calif.), using CDFDUETFOR (5′-agctccatgggaagcacacggtcacactgct-3′, SEQ ID NO:62) and CDFDUETREV (5′-agctgcatgcaagttagctcactcattaggga-3′, SEQ ID NO:63), digested with SphI and NcoI (these sites are underlined in the primer sequences), and then inserted into the SphI and BspHI sites in pTrc and its derivatives. All gpD-encoding plasmids containing the pBR322 or CDF origins of replication also carry the ampicillin or spectinomycin antibiotic resistance genes, respectively.

Lysogens of TOP10 cells (Invitrogen, Carlsbad, Calif.) containing λ D1180 (Luc) (Eguchi, A., et al. (2001)) were transformed with pTrc plasmids encoding either wild-type gpD alone, gpD-3JCLI4 alone, or the combination of wild-type gpD plus gpD-3JCLI4. Lysogens containing the coat protein plasmids were grown to mid-log phase at 32° C. in the presence of antibiotics (ampicillin or spectinomycin, 50 μg/mL, Sigma). The lysogens, which contain a temperature-sensitive mutation in the cI repressor, were then induced by increasing the culture temperature to 45° C. for approximately 15 minutes. After induction, cultures were incubated at 38° C. for an additional 3 hours to allow for phage replication and assembly. Cells were then collected by centrifugation and lysed with chloroform (Sigma). DNAse I (Worthington Biochemical Corporation, Lakewood, N.J.) was added to a final concentration of 10 μg/mL to remove any contaminating nucleic acids, and lysed cultures were cleared of debris by centrifugation; phage were pelleted from the supernatant by ultracentrifugation. The resulting pellet was resuspended in phage suspension media (100 mM NaCl, 10 mM MgSO4, 50 mM Tris-Cl (pH 7.5), 0.1% gelatin) and further purified by cesium chloride density gradient ultracentrifugation. The resulting phage bands were pulled from the gradient using a syringe and 18 G needle and dialyzed against 10 mM NaCl, 50 mM Tris-Cl (pH 7.5), and 10 mM MgCl2. Phage preparations were titered on LE392 (supE, supF) E. coli host cells (Stratagene, La Jolla, Calif.).

The authentic (non codon-optimized) λ phage gpD gene was amplified by PCR from pAT101 (Yang, F., et al. (2000); Forrer, P. and Jaussi, R. (1998)) using the gpD forward primer (5′-ggtgcccatatggcgagcaaagaaacctttacc-3′, SEQ ID NO:64) and the gpD reverse primer (5′-ccgacgggatcctcattaaacgatgctgattgc-3′, SEQ ID NO:65) and then cloned into the pET15b plasmid (Novagen, San Diego, Calif.) using the NdeI and BamHI restriction sites (underlined). The resulting pET15b-gpD plasmid was transformed into BL21 cells (Invitrogen, Carlsbad, Calif.). Transformed bacteria were grown to mid-log phase and then induced for 3 hours with IPTG added to a final concentration of 1 mM. Cells were pelleted by centrifugation, lysed with BugBuster (Novagen, San Diego, Calif.), and treated with BENZONASE® and lysozyme (Novagen, San Diego, Calif.) according to the manufacturer's recommendations. The resulting lysate was clarified by centrifugation and temporarily stored at −20° C. Thawed lysate was purified using a Co²⁺ column (BD TALON™; BD Biosciences Clontech, Mountain View, Calif.) according to the manufacturer's recommendations. The eluted fractions were analyzed by polyacrylamide gel electrophoresis (PAGE), followed by immunoblot analysis using an anti-His5 antibody reactive with the His6-tag that had been added to the gpD protein. Approximately 3 mg of PAGE purified gpD protein was used to raise a polyclonal antiserum in rabbits (Sigma Genosys, The Woodlands, Tex.). The reactivity of the resulting antiserum against gpD was confirmed by immunoblot analysis, and the antiserum was then preserved in 0.02% sodium merthiolate and stored in aliquots at −80° C. until use.

1×10⁹ plaque forming units (PFU) of CsCl-banded phage particles were combined with 2×SDS loading buffer and heated to 95° C. for 5 minutes. Samples were separated on a 20% SDS-PAGE gel. Proteins were then transferred to a nitrocellulose membrane and incubated with the polyclonal anti-gpD anti-serum at a dilution of 1:1000 (in 1×PBS/0.1% Tween (PBST) containing 5% nonfat dry milk). After washing with PBST, the nitrocellulose membrane was incubated with HRP-conjugated donkey anti-rabbit antibody (Amersham, Piscataway, N.J.) at a dilution of 1:3000 (also in PBST containing 5% nonfat dry milk). HRP-conjugated antibody was detected using ECL-Plus substrate (Amersham, Piscataway, N.J.). Blots were imaged using the ChemiDoc XRS chemiluminescence chamber and Quantity One software version 4.5.2 (BioRad, Hercules, Calif.).

In order to generate plasmids capable of efficiently expressing gpD in E. coli host cells, an E. coli codon-optimized (ECO) derivative of the wild-type λ gpD gene was synthesized. The E. coli codon-optimized gene has greatly reduced homology to its native λ counterpart (only 78% nucleotide identity), which can effectively eliminate the potential for homologous DNA recombination between the ECO-gpD sequence and its native λ phage counterpart, thereby reducing the possibility that a complementing gpD plasmid might recombine with a gpD-deficient λ lysogen.

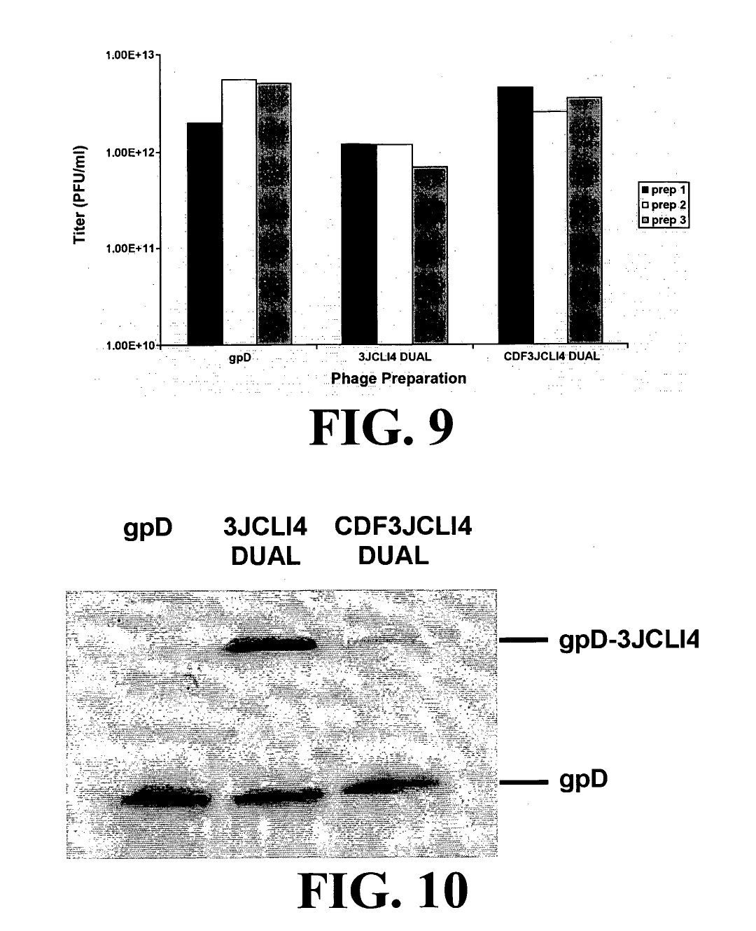

The ECO-gpD gene was inserted into pTrc to create pTrc-gpD (FIG. 7A), and a sequence cassette corresponding to the desired fusion partner (3JCLI4) was then inserted as a C-terminal fusion to gpD, to create pTrc-gpD-3JCLI4 (FIG. 7B). Additionally, constructs were generated in which the pBR322 origin of replication in the pTrc-based vectors was replaced with the CDF origin of replication, to yield pTrc-gpD-CDF (FIG. 7C) and pTrc-gpD-3JCLI4-CDF (FIG. 7D). Protein expression from each of these constructs was confirmed by immunoblot analysis of IPTG-induced bacterial cell lysates using a gpD-specific rabbit antiserum; the constructs were then transformed into λ D1180 lysogens, in order to examine their ability to complement this gpD-deficient phage strain and thereby permit recovery of infectious phage particles.

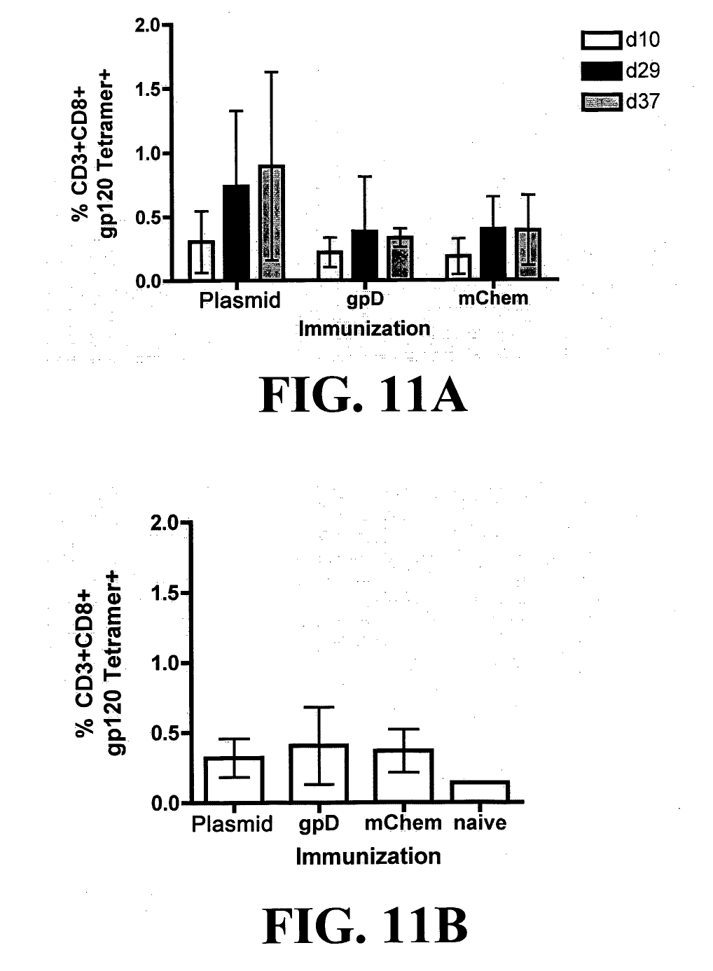

Lysogens of TOP10 cells containing λ D1180 (Luc) were transformed with either the pTrc-gpD-3JCLI4 or pTrc-gpD expression plasmid, encoding the gpD-3JCLI4 fusion protein or wild-type gpD, respectively. Following heat induction, cell lysis and cesium chloride density gradient ultracentrifugation of a 1 liter preparation, no phage particles could be recovered from the lysogens that had been transformed with the plasmid vector encoding the recombinant gpD-3JCLI4 coat protein fusion, as reflected by the absence of the characteristic λ phage band in the cesium chloride density gradient (FIG. 8B). In contrast, when the λ D1180 (Luc) lysogens were transformed with a plasmid encoding wild-type gpD, infectious phage particles were readily recovered (FIG. 8A).

These results indicated that the gpD-3JCLI4 fusion protein either interfere with phage assembly or prevent the formation of stable phage particles. To resolve this unexpected difficulty, the gpD-3JCLI4 fusion protein was expressed on the surface of phage λ through the use of a mosaic approach, in which the final virion contained a mixture of both wild-type and recombinant gpD subunits.

Lysogens of TOP10 cells containing λ D1180 (Luc) were co-transformed with plasmids encoding both recombinant and wild-type forms of gpD. This was achieved using a set of gpD expression vectors that contained one of two origins of replication—either the pBR322-derived pMB1 origin (pBR-ori) or a second compatible, low-copy origin derived from pCDF-1b (CDF-ori). Lysogens were then cotransformed with either (i) a pBR-ori based plasmid encoding a recombinant form of gpD (pTrc-gpD-3JCLI4) plus a CDF-ori based plasmid encoding wild-type gpD (pTrc-gpD-CDF), or (ii) a CDF-ori based plasmid encoding a recombinant form of gpD (pTrc-gpD-3JCL14-CDF) plus a pBR-ori based plasmid encoding wild-type gpD (pTrc-gpD). Following heat induction of the lysogen, cell lysis, and cesium chloride density gradient ultracentrifugation, phage containing the gpD-3JCLI4 fusion protein that had previously proven recalcitrant was efficiently recovered, as reflected by the presence of a characteristic phage band in the CsCl gradient (FIGS. 8C, D).

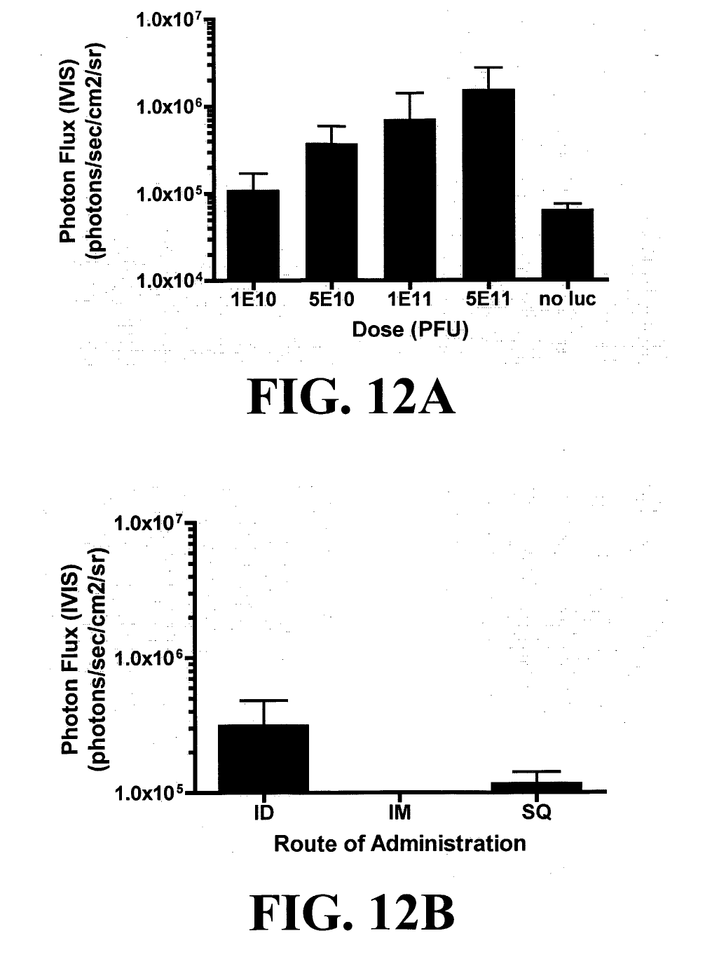

These recombinant phage preparations were collected from the CsCl gradient, dialyzed, and titered on LE392 E. coli host cells. All of the phage preparations, including the mosaic phages were found to be infectious (FIG. 9). The titers of the mosaic phages were generally similar to those of wild-type phage preparations (i.e., gpD-deficient phage that were complemented using wild-type gpD alone), although it was noted that use of the pBR-based gpD fusion constructs resulted in somewhat lower titers of the mosaic phage, as compared to their CDF-based counterparts (compare titers for 3JCLI4 DUAL with those for CDF3JCLI4 DUAL; FIG. 9).

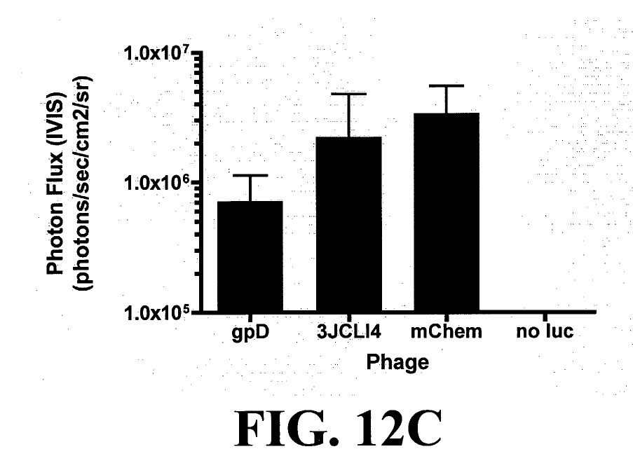

An aliquot (1×10⁹ PFU) of each of these phage preparations was extracted, separated on a 20% SDS-PAGE gel, and subjected to immunoblot analysis using the rabbit polyclonal antiserum against gpD. As expected, the phage preparations that were derived from lysogens that had been cotransformed with wild-type and recombinant gpD expression plasmids contained two distinct forms of gpD—consistent with their being gpD-mosaic phages (compare results for gpD wild-type to those for the other constructs shown in FIG. 10). It was also noted that use of the pBR-based gpD fusion constructs resulted in slightly greater levels of recombinant gpD into the phage particles, as compared to the CDF-based plasmids (compare results for 3JCL14 DUAL with those for CDF3JCLI4 DUAL; FIG. 10).

### Example 5

### Lambda Phage Elicit Immune Response

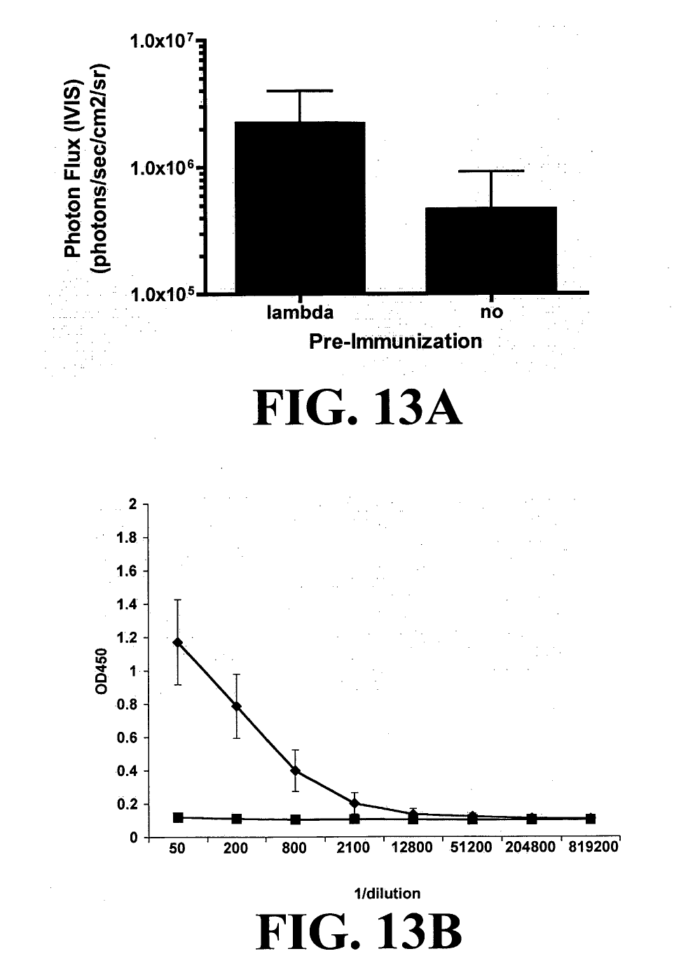

Six week old female BALB/c mice were immunized intradermally (ID), via the tailbase, in groups of 4 with 1×10¹¹ PFU gpD or mChem (gp120) phage, or 100 μg gp120-encoding plasmid DNA as a positive control (all immunizations were in 50-100 μL total volume). Fourteen and 30 days following the initial immunization, mice in the phage and plasmid DNA groups were boosted with using a homologous prime-boost approach. Ten, 29, and 37 days following immunization, whole blood was used for tetramer staining with APC (allophyocyanin) conjugated H-2D_{d} tetramers loaded with the HIV-1 gp120 V3 peptide (RGPGRAFVTI; SEQ ID NO:66) (FIG. 11A). Mice were sacrificed at day 38 and splenocytes were used for gp120 tetramer staining (FIG. 11B). The results show that phage-immunized mice mount an immune response to a phage-encoded protein.

### Example 6

### Lambda Phage-Mediated Luciferase Expression is Dose-Dependent, and Also Depends on the Route of Phage Delivery; Surface Modifications of the Phage Result in Improved Gene Expression

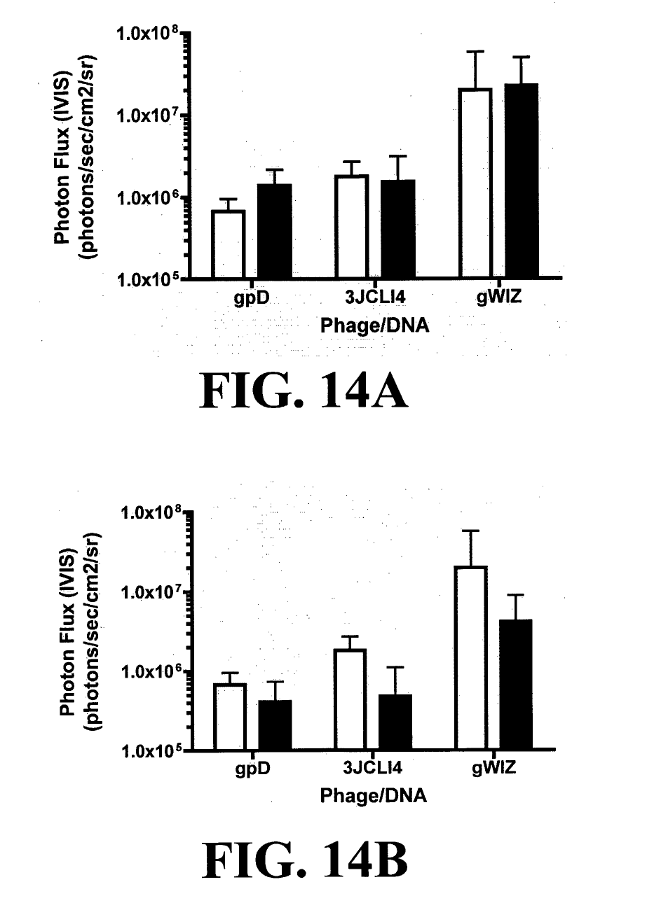

Eight to twelve week old female BALB/c mice were injected intradermally (ID), via the tailbase, with either 1×10¹⁰, 5×10¹⁰, 1×10¹¹, or 5×10¹¹ PFU of gpD (luc) phage or 1×10¹¹ PFU of gpD (no luc) phage (A). Twenty-four hours later, luciferase expression was measured using the Xenogen IVIS system. In vivo luciferase gene expression was found to be dose dependent in mice injected ID with gpD (luc) phage (FIG. 12A). In FIG. 12B, eight to twelve week old female BALB/c mice were injected intradermally (ID), via the tailbase, intramuscularly (IM) in the thigh, or subcutaneously (SQ), in groups of 8 with 1×10¹¹ PFU gpD (Luc) phage. Four mice were injected ID, IM, or SQ with 1×10¹¹ PFU phage that lacked the luciferase expression cassette (no Luc) as a negative control for each group. Mice were imaged for luciferase expression. In vivo luciferase gene expression was evident in mice injected ID with gpD (Luc) phage (FIG. 12B), but weaker when phage was delivered via other routes. In FIG. 12C, eight to twelve week old female BALB/c mice were injected ID in groups of 8 with 1×10¹¹ PFU of either wild-type gpD phage, 3JCLI4 phage (phage containing a modified gpD protein bearing the 3JCL4 integrin-binding peptide), or chemerin (mChem) phage (phage containing a modified gpD protein containing a chemerin receptor-binding peptide). Four mice were injected with 1×10¹¹ PFU of phage that lacked the luciferase expression cassette (no Luc) as a negative control. In vivo luciferase gene expression was found to be greater in animals injected with 3JCLI4 or chemerin targeted (C, n=8, p<0.05 one-way ANOVA, Tukey's post-test).

### Example 7

### Pre-Existing Immunity to Lambda Phage Results in Increased Gene Expression

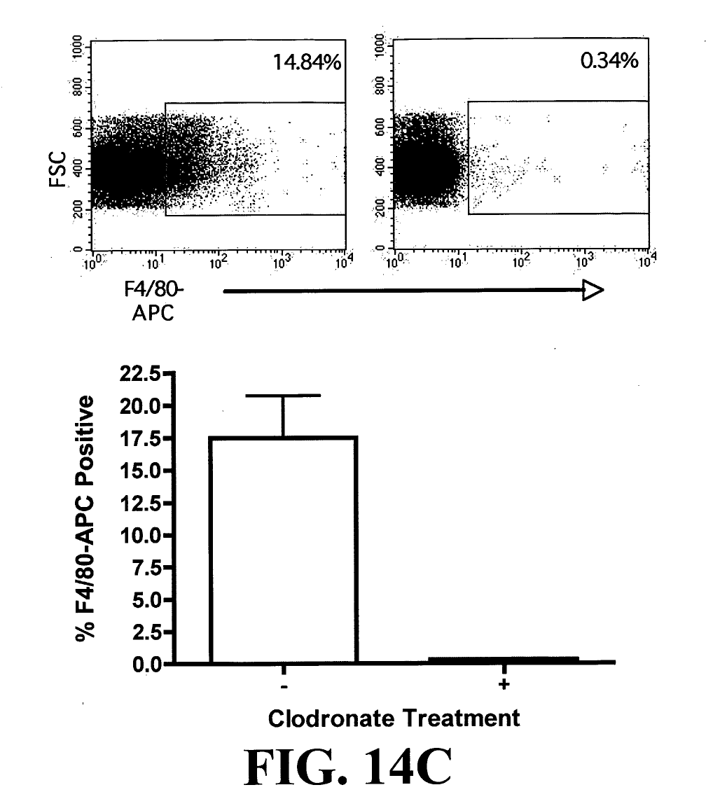

Eight to twelve week old female BALB/c mice were first immunized IM with either 1×10¹¹ PFU of gpD (no luc) bacteriophage lambda (1) in 50 μL of suspension media or 50 μL of suspension media alone (no). Two weeks post-immunization, all mice were injected ID, via the tailbase, with 1×10¹¹ PFU of gpD (luc) phage. Twenty-four hours later, mice were imaged for luciferase expression. There was a statistically significant difference in in vivo luciferase gene expression between mice that were pre-immunized with bacteriophage lambda versus mice that were pre-immunized with suspension media alone (p<0.05, Student's two-tailed t-test; FIG. 13A). Sera from mice was collected two weeks post-immunization and analyzed by ELISA. Antibodies specific for bacteriophage lambda were detected in mice pre-immunized with bacteriophage lambda (FIG. 13B).

### Example 8

### Lambda Phage-Mediated Gene Expression is Partially Dependent on the Presence of Phagocytic Cells, But is not Enhanced by Chloroquine

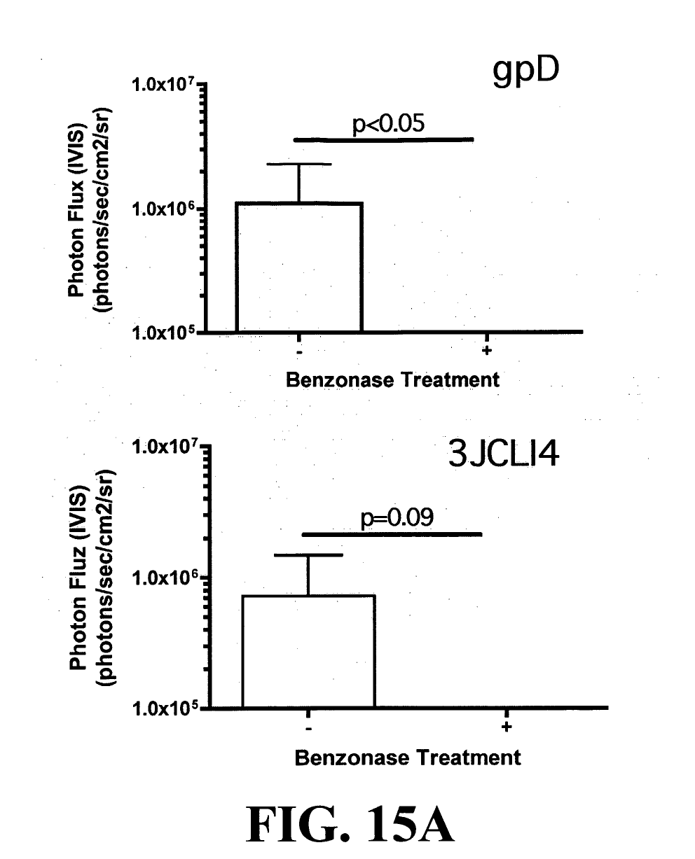

Eight to twelve week old female BALB/c mice were injected IP with 2 mg per mouse of chloroquine or were left untreated. Two hours following chloroquine injection, 1×10¹¹ PFU of either wild-type gpD (luc) phage, 3JCLI4 (luc) phage, or 100 μg of gWIZ plasmid DNA (a plasmid vector encoding luciferase under the transcriptional control of the CMV promoter) was injected ID, via the tailbase. Twenty-four hours later, mice were imaged for luciferase expression. No difference in in vivo luciferase gene expression was observed in animals treated with chloroquine prior to phage or DNA injection (FIG. 14A). In addition, eight to twelve week old female BALB/c mice were injected in groups of 4 with clodronate liposomes via a combined IP (200 μl) and ID (100 μl) route (shaed bars) or were left untreated (open bars). Forty-eight hours following clodronate liposome injection, 1×10¹¹ PFU of either wild-type gpD (luc) phage, 3JCLI4 (luc) phage, or 100 μg of gWIZ (luc) plasmid DNA was injected ID, via the tailbase. A decrease in in vivo luciferase gene expression in animals treated with clodronate liposomes prior to phage or DNA injection was observed (FIG. 14B). After imaging, mice were sacrificed and splenocytes were stained for F4/80 (a cell surface marker for macrophages). Mice that received clodronate liposomes had a decrease in F4/80 positive splenocytes, as measured by flow cytometric analysis, showing that phagocytic cells were depleted in these mice (FIG. 14C).

### Example 9

### Lambda-Phage Mediated Gene Expression is Dependent on DNA Bound to the Surface of the Phage Particle

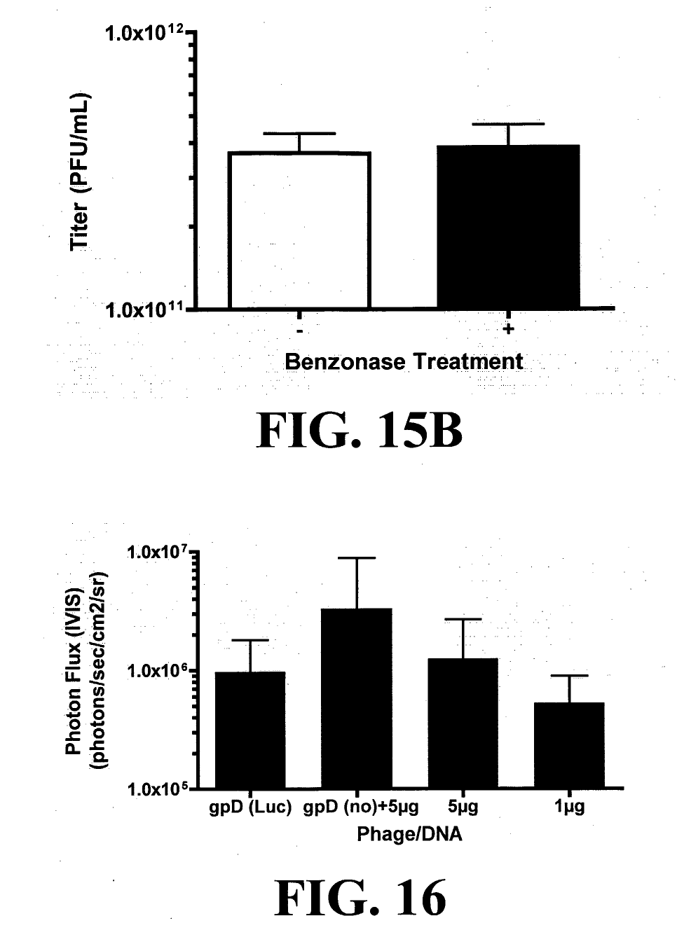

Eight to twelve week old female BALB/c mice were injected in groups of 5 with 1×10¹¹ PFU of either wild-type gpD (luc) phage or 3JCLI4 (luc) phage. In FIG. 15A, phage was either treated with 50 U of benzonase (a DNA-degrading enzyme) or buffer alone prior to ID injection, via the tailbase route. Twenty-four hours later, mice were imaged for luciferase expression. Mice receiving benzonase treated phage did not have a detectable luciferase signal, whereas mice receiving untreated phage had strong luciferase expression (FIG. 15A). In addition, 6×10¹¹ PFU of gpD (luc) phage was treated with 50 U or benzonase or buffer alone (50 μL total volume), and the treated phage were then re-titered on LE392 E. coli cells to determine if benzonase treatment affected phage viability. Phage titers were unaltered by benzonase treatment, indicating that benzonase treatment does not lead to loss of phage viability or damage to phage particles (FIG. 15B).

In a follow-up study, eight to twelve week old female BALB/c mice were injected in groups of 8 with either (i) 1×10¹¹ PFU of wild-type gpD (luc) phage, (ii) 1×10¹¹ PFU of wild-type phage containing no mammalian expression cassette but physically mixed with 5 μg of purified lambda phage DNA that was isolated from the luciferase-encoding phage (λ luc DNA), (iii) 5 μg of purified λ luc DNA, or (iv) 1 μg of λ luc DNA. Twenty-four hours later, mice were imaged for luciferase expression. In vivo luciferase expression in BALB/c mice injected with gpD (luc) was similar to expression levels obtained from 1 and 5 μg of purified λ luc DNA alone, indicating that an aliquot of 1×10¹¹ PFU of CsCl-gradient purified phage particles contains between 1 and 5 μg of external, surface-bound phage DNA.

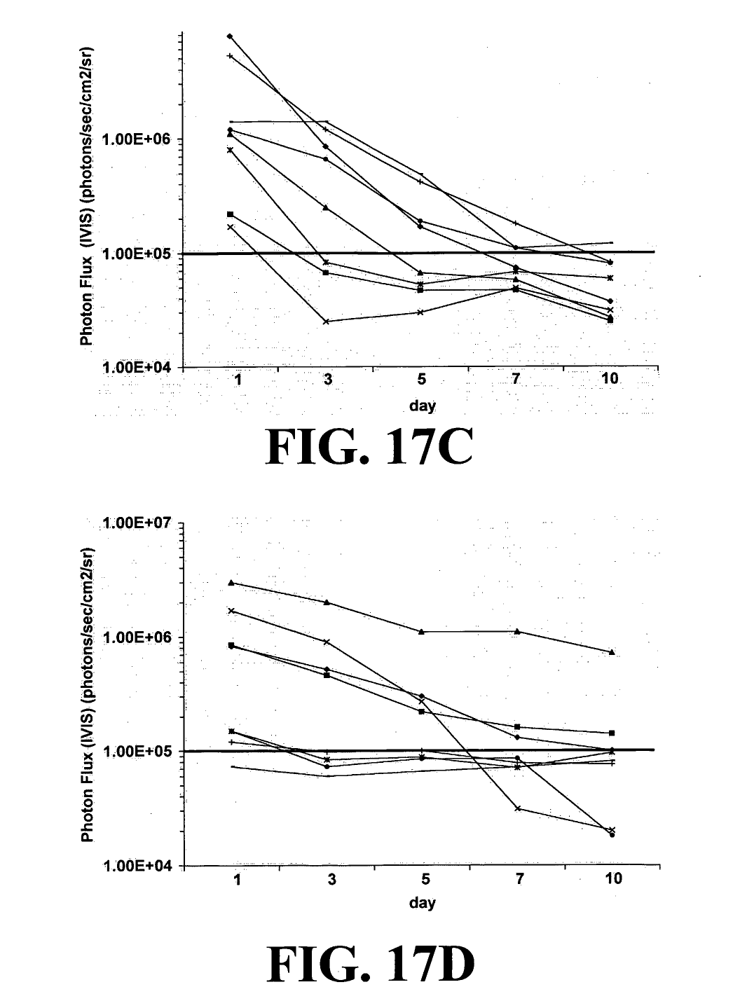

In a second follow study, eight to twelve week old female BALB/c mice were injected in groups of 8 with either 1×10¹¹ PFU of wild-type gpD (luc) phage (FIGS. 17A, 17C) or 5 μg of purified λ luc DNA (FIGS. 17B, 17D). Luciferase expression was then measured over time. In vivo luciferase expression in mice injected with gpD (luc) was found to show a similar time-course to similar to luciferase expression in mice injected with 5 μg purified λ luc DNA.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

Aderem, A., and Underhill, D. M. 1999. Mechanisms of phagocytosis in macrophages. Annu Rev Immunol 17:593-623.
Akuta, T., Eguchi, A., Okuyama, H., Senda, T., Inokuchi, H., Suzuki, Y., Nagoshi, E., Mizuguchi, H., Hayakawa, T., Takeda, K. et al. (2002) Enhancement of phage-mediated gene transfer by nuclear localization signal. Biochem Biophys Res Commun, 297, 779-786.
Amara, R. R., F. Villinger, J. D. Altman, S. L. Lydy, S. P. O'Neil, S. I. Staprans, D. C. Montefiori, Y. Xu, J. G. Herndon, L. S. Wyatt, M. A. Candido, N. L. Kozyr, P. L. Earl, J. M. Smith, H. L. Ma, B. D. Grimm, M. L. Hulsey, J. Miller, H. M. McClure, J. M. McNicholl, B. Moss, and H. L. Robinson. 2001. Control of a mucosal challenge and prevention of AIDS by a multiprotein DNA/MVA vaccine. Science 292:69-74.
Anderson H A, Chen Y, Norkin L C. 1996. Bound simian virus 40 translocates to caveolin-enriched membrane domains, and its entry is inhibited by drugs that selectively disrupt caveolae. Mol Biol Cell 7:1825-1834.
Arap, W., Pasqualini, R. and Ruoslahti, E. (1998) Cancer treatment by targeted drug delivery to tumor vasculature in a mouse model. Science, 279, 377-380.
Baorto, D. M., Z. Gao, R. Malaviya, M. L. Dustin, A. van der Merwe, D. M. Lublin, and S. N. Abraham. 1997. Survival of FimH-expressing enterobacteria in macrophages relies on glycolipid traffic. Nature 389:636-9.
Barbas, C. F., 3rd, Kang, A. S., Lerner, R. A. and Benkovic, S. J. (1991) Assembly of combinatorial antibody libraries on phage surfaces: the gene III site. Proc Natl Acad Sci USA, 88, 7978-7982.
Barouch, D. H., S. Santra, J. E. Schmitz, M. J. Kuroda, T. M. Fu, W. Wagner, M. Bilska, A. Craiu, X. X. Zheng, G. R. Krivulka, K. Beaudry, M. A. Lifton, C. E. Nickerson, W. L. Trigona, K. Punt, D. C. Freed, L. Guan, S. Dubey, D. Casimiro, A. Simon, M. E. Davies, M. Chastain, T. B. Strom, R. S. Gelman, D. C. Montefiori, M. G. Lewis, E. A. Emini, J. W. Shiver, and N. L. Letvin. 2000. Control of viremia and prevention of clinical AIDS in rhesus monkeys by cytokine-augmented DNA vaccination. Science 290:486-92.
Barrow, P. A., and J. S. Soothill. 1997. Bacteriophage therapy and prophylaxis: rediscovery and renewed assessment of potential. Trends Microbiol 5:268-71.
Barrow, P., M. Lovell, and A. Berchieri, Jr. 1998. Use of lytic bacteriophage for control of experimental Escherichia coli septicemia and meningitis in chickens and calves. Clin Diagn Lab Immunol 5:294-8.
Becerril, B., Poul, M. A. and Marks, J. D. (1999) Toward selection of internalizing antibodies from phage libraries. Biochem Biophys Res Commun, 255, 386-393.
Berchtold S, Muhl-Zurbes P, Maczek E, Golka A, Schuler G, Steinkasserer A. 2002. Cloning and characterization of the promoter region of the human CD83 gene. Immunobiology 205:231-246.
Bilgrami, S., S. Tomar, S. Yadav, P. Kaur, J. Kumar, T. Jabeen, S. Sharma, and T. P. Singh. 2004. Crystal structure of schistatin, a disintegrin homodimer from saw-scaled viper (Echis carinatus) at 2.5 A resolution. J Mol Biol 341:829-37.
Bolivar, F., Rodriguez, R. L., Greene, P. J., Betlach, M. C., Heyneker, H. L. and Boyer, H. W. (1977) Construction and characterization of new cloning vehicles. II. A multipurpose cloning system. Gene, 2, 95-113.
Bonifaz L. C., et al. 2004. In vivo targeting of antigens to maturing dendritic cells via the DEC-205 receptor improves T cell vaccination. J Exp Med 199:815-824.
Boratynski, J., D. Syper, B. Weber-Dabrowska, M. Lusiak-Szelachowska, G. Pozniak, and A. Gorski. 2004. Preparation of endotoxin-free bacteriophages. Cell Mol Biol Lett 9:253-9.
Bradel-Tretheway, B. G., Zhen, Z. and Dewhurst, S. (2003) Effects of codon-optimization on protein expression by the human herpesvirus 6 and 7 U51 open reading frame. J Virol Methods, 111, 145-156.
Brocker T, Riedinger M, Kaalainen K. 1997. Targeted expression of major histocompatibility complex (MHC) class II molecules demonstrates that dendritic cells can induce negative but not positive selection of thymocytes in vivo. J Exp Med 185:541-550.
Brosius, J., Cate, R. L. and Perlmutter, A. P. (1982) Precise location of two promoters for the beta-lactamase gene of pBR322. S1 mapping of ribonucleic acid isolated from Escherichia coli or synthesized in vitro. J Biol Chem, 257, 9205-9210.
Burg, M. A., K. Jensen-Pergakes, A. M. Gonzalez, P. Ravey, A. Baird, and D. Larocca. 2002. Enhanced phagemid particle gene transfer in camptothecin-treated carcinoma cells. Cancer Res 62:977-81.
Chauthaiwale, V. M., A. Therwath, and V. V. Deshpande. 1992. Bacteriophage lambda as a cloning vector. Microbiol Rev 56:577-91.
Chen, K., S. Braun, S. Lyman, Y. Fan, C. M. Traycoff, E. A. Wiebke, J. Gaddy, G. Sledge, H. E. Broxmeyer, and K. Cometta. 1997. Antitumor activity and immunotherapeutic properties of Flt3-ligand in a murine breast cancer model. Cancer Research 57:3511-6.
Chen, X., G. Scala, I. Quinto, W. Liu, T. W. Chun, J. S. Justement, O. J. Cohen, T. C. vanCott, M. Iwanicki, M. G. Lewis, J. Greenhouse, T. Barry, D. Venzon, and A. S. Fauci. 2001. Protection of rhesus macaques against disease progression from pathogenic SHIV-89.6PD by vaccination with phage-displayed HIV-1 epitopes. Nat Med 7:1225-31.
Clackson, T., Hoogenboom, H. R., Griffiths, A. D. and Winter, G. (1991) Making antibody fragments using phage display libraries. Nature, 352, 624-628.
Clark, J. R., and J. B. March. 2004. Bacteriophage-mediated nucleic acid immunisation. FEMS Immunol Med Microbiol 40:21-6.
Clark, K. R., and P. R. Johnson. 2001. Gene delivery of vaccines for infectious disease. Curr Opin Mol Ther 3:375-84.
Cloutier, S. M., S. Couty, A. Terskikh, L. Marguerat, V. Crivelli, M. Pugnieres, J. C. Mani, H. J. Leisinger, J. P. Mach, and D. Deperthes. 2000. Streptabody, a high avidity molecule made by tetramerization of in vivo biotinylated, phage display-selected scFv fragments on streptavidin. Mol Immunol 37:1067-77.
Console, S., C. Marty, C. Garcia-Echeverria, R. Schwendener, and K. Ballmer-Hofer. 2003. Antennapedia and HIV transactivator of transcription (TAT) “protein transduction domains” promote endocytosis of high molecular weight cargo upon binding to cell surface glycosaminoglycans. J Biol Chem 278:35109-14. Epub 2003 Jun. 30.
Cooper, C. L., H. L. Davis, M. L. Morris, S. M. Efler, A. M. Krieg, Y. Li, C. Laframboise, M. J. Al Adhami, Y. Khaliq, I. Seguin, and D. W. Cameron. 2004. Safety and immunogenicity of CPG 7909 injection as an adjuvant to Fluarix influenza vaccine. Vaccine 22:3136-43.
Cortese, R., Felici, F., Galfre, G., Luzzago, A., Monaci, P. and Nicosia, A. (1994) Epitope discovery using peptide libraries displayed on phage. Trends Biotechnol, 12, 262-267.
Cotten, M., A. Baker, M. Saltik, E. Wagner, and M. Buschle. 1994. Lipopolysaccharide is a frequent contaminant of plasmid DNA preparations and can be toxic to primary human cells in the presence of adenovirus. Gene Ther 1:239-46.
Cui Y, Golob J, Kelleher E, Ye Z, Pardoll D, Cheng L. 2002. Targeting transgene expression to antigen-presenting cells derived from lentivirus-transduced engrafting human hematopoietic stem/progenitor cells. Blood 99:399-408.
De Berardinis, P., L. D'Apice, A. Prisco, M. N. Ombra, P. Barba, G. Del Pozzo, S. Petukhov, P. Malik, R. N. Perham, and J. Guardiola. 1999. Recognition of HIV-derived B and T cell epitopes displayed on filamentous phages. Vaccine 17:1434-41.
De Berardinis, P., Sartorius, R., Fanutti, C., Perham, R. N., Del Pozzo, G. and Guardiola, J. (2000) Phage display of peptide epitopes from HIV-1 elicits strong cytolytic responses. Nat Biotechnol, 18, 873-876.
Decatur, A. L., and D. A. Portnoy. 2000. A PEST-like sequence in listeriolysin O essential for Listeria monocytogenes pathogenicity. Science 290:992-5.
Degano, P., J. Schneider, C. M. Hannan, S. C. Gilbert, and A. V. Hill. 1999. Gene gun intradermal DNA immunization followed by boosting with modified vaccinia virus Ankara: enhanced CD8+ T cell immunogenicity and protective efficacy in the influenza and malaria models. Vaccine 18:623-32.
Derossi, D., A. H. Joliot, G. Chassaing, and A. Prochiantz. 1994. The third helix of the Antennapedia homeodomain translocates through biological membranes. J Biol Chem 269:10444-50.
Di Giovine, M., B. Salone, Y. Martina, V. Amati, G. Zambruno, E. Cundari, C. M. Failla, and I. Saggio. 2001. Binding properties, cell delivery, and gene transfer of adenoviral penton base displaying bacteriophage. Virology 282:102-12.
Dubos, R., J. Straus, and C. Pierce. 1943. The multiplication of bacteriophage in vivo and its protective effect against an experimental infection with Shigella dysenteriae. Journal of Experimental Medicine 78:161-168.
Duerr, D. M., S. J. White, and H. J. Schluesener. 2004. Identification of peptide sequences that induce the transport of phage across the gastrointestinal mucosal barrier. J Virol Methods 116:177-80.
Dutta, S., L. A. Ware, A. Barbosa, C. F. Ockenhouse, and D. E. Lanar. 2001. Purification, characterization, and immunogenicity of a disulfide cross-linked Plasmodium vivax vaccine candidate antigen, merozoite surface polypeptide 1, expressed in Escherichia coli. Infect Immun 69:5464-70.
Eguchi, A., Furusawa, H., Yamamoto, A., Akuta, T., Hasegawa, M., Okahata, Y. and Nakanishi, M. (2005) Optimization of nuclear localization signal for nuclear transport of DNA-encapsulating particles. J Control Release, 104, 507-519.
Eguchi, A., T. Akuta, H. Okuyama, T. Senda, H. Yokoi, H. Inokuchi, S. Fujita, T. Hayakawa, K. Takeda, M. Hasegawa, and M. Nakanishi. 2001. Protein transduction domain of HIV-1 Tat protein promotes efficient delivery of DNA into mammalian cells. J Biol Chem 276:26204-10.
Empig, C. J., and Goldsmith, M. A. 2002. Association of the caveola vesicular system with cellular entry by filoviruses. J Virol 76:5266-5270.
Fanutti, C., Del Pozzo, G., De Berardinis, P., Guardiola, J., Deng, L. W. and Perham, R. N. (1998) Phage-display of antigenic peptides applied to vaccine design. Biochem Soc Trans, 26, S8.
Folgori, A., Tafi, R., Meola, A., Felici, F., Galfre, G., Cortese, R., Monaci, P. and Nicosia, A. (1994) A general strategy to identify mimotopes of pathological antigens using only random peptide libraries and human sera. Embo J, 13, 2236-2243.
Forrer, P. and Jaussi, R. (1998) High-level expression of soluble heterologous proteins in the cytoplasm of Escherichia coli by fusion to the bacteriophage lambda head protein D. Gene, 224, 45-52.
Freund C, Ross A, Guth B, Pluckthun A, Holak T A. 1993. Characterization of the linker peptide of the single-chain Fv fragment of an antibody by NMR spectroscopy. FEBS Lett 320:97-100.
Fuglsang, A. 2004. Codon optimizer: a freeware tool for codon optimization. Protein Expr Purif 311; 247-249.
Galfre, G., Monaci, P., Nicosia, A., Luzzago, A., Felici, F. and Cortese, R. (1996) Immunization with phage-displayed mimotopes. Methods Enzymol, 267, 109-115.
Gao W, Rzewski A, Sun H, Robbins P D, Gambotto A. 2004. UpGene: Application of a web-based DNA codon optimization algorithm. Biotechnol Prog 20:443-448.
Geijtenbeek T B, Kwon D S, Torensma R, van Vliet S J, van Duijnhoven G C, Middel J, Cornelissen I L, Nottet H S, KewalRamani V N, Littman D R, Figdor C G, van Kooyk Y. 2000. DC-SIGN, a dendritic cell-specific HIV-1-binding protein that enhances trans-infection of T cells. Cell 100:587-597.
Geijtenbeek T B, Torensma R, van Vliet S J, van Duijnhoven G C, Adema G J, van Kooyk Y, Figdor C G. 2000. Identification of DC-SIGN, a novel dendritic cell-specific ICAM-3 receptor that supports primary immune responses. Cell 100:575-585.
Gough P. J. and Raines, E. W. Gene therapy of apolipoprotein E-deficient mice using a novel macrophage-specific retroviral vector. 2003. Blood 101:485-491.
Gratton, J. P., J. Yu, J. W. Griffith, R. W. Babbitt, R. S. Scotland, R. Hickey, F. J. Giordano, and W. C. Sessa. 2003. Cell-permeable peptides improve cellular uptake and therapeutic gene delivery of replication-deficient viruses in cells and in vivo. Nat Med 9:357-62.
Grifman M, Trepel M, Speece P, Gilbert L B, Arap W, Pasqualini R, Weitzman M D. 2001. Incorporation of tumor-targeting peptides into recombinant adeno-associated virus capsids. Mol Ther 3:964-975.
Grosjean, H. and Fiers, W. 1982. Preferential codon usage in prokaryotic genes: the optimal codon-anticodon interaction energy and the selective codon usage in efficiently expressed genes. Gene 18:199-209.
Gupta, A., M. Onda, I. Pastan, S. Adhya, and V. K. Chaudhary. 2003. High-density functional display of proteins on bacteriophage lambda. J Mol Biol 334:241-54.
Gupta, A., Onda, M., Pastan, I., Adhya, S. and Chaudhary, V. K. (2003) High-density functional display of proteins on bacteriophage lambda. J Mol Biol, 334, 241-254.
Halstead, S. B. 2003. Neutralization and antibody-dependent enhancement of dengue viruses. Advances in Virus Research 60:421-67.
Han, X., J. H. Bushweller, D. S. Cafiso, and L. K. Tamm. 2001. Membrane structure and fusion-triggering conformational change of the fusion domain from influenza hemagglutinin. Nat Struct Biol 8:715-20.
Hellwig, S. M., H. F. van Oirschot, W. L. Hazenbos, A. B. van Spriel, F. R. Mooi, and J. G. van De Winkel. 2001. Targeting to Fcgamma receptors, but not CR3 (CD11b/CD18), increases clearance of Bordetella pertussis. J Infect Dis 183:871-9. Epub 2001 Feb. 13.
Henning, P., M. K. Magnusson, E. Gunneriusson, S. S. Hong, P. Boulanger, P. A. Nygren, and L. Lindholm. 2002. Genetic modification of adenovirus 5 tropism by a novel class of ligands based on a three-helix bundle scaffold derived from staphylococcal protein A. Hum Gene Ther 13:1427-39.
Hildebrand, G. J., and H. Wolochow. 1962. Translocation of bacteriophage across the intestinal wall of the rat. Proc Soc Exp Biol Med 109:183-5.
Hoess, R. H. (2002) Bacteriophage lambda as a vehicle for peptide and protein display. Curr Pharm Biotechnol, 3, 23-28.
Hunger R E, Sieling P A, Ochoa M T, Sugaya M, Burdick A E, Rea T H, Brennan P J, Belisle J T, Blauvelt A, Porcelli S A, Modlin R L. 2004. Langerhans cells utilize CD1a and langerin to efficiently present nonpeptide antigens to T cells. J Clin Invest 113:701-708.
Jepson, C. D., and J. B. March. 2004. Bacteriophage lambda is a highly stable DNA vaccine delivery vehicle. Vaccine 22:2413-9.
Jiang, W. et al. 1995. The receptor DEC-205 expressed by dendritic cells and thymic epithelial cells is involved in antigen processing. Nature 375:151-155.
Jones, J. I., T. Prevette, A. Gockerman, and D. R. Clemmons. 1996. Ligand occupancy of the alpha-V-beta3 integrin is necessary for smooth muscle cells to migrate in response to insulin-like growth factor. Proc Natl Acad Sci USA 93:2482-7.
Juliano, D., Y. Wang, C. Marcinkiewicz, L. A. Rosenthal, G. J. Stewart, and S. Niewiarowski. 1996. Disintegrin interaction with alpha V beta 3 integrin on human umbilical vein endothelial cells: expression of ligand-induced binding site on beta 3 subunit. Exp Cell Res 225:132-42.
Katz, B. A. 1995. Binding to protein targets of peptidic leads discovered by phage display: crystal structures of streptavidin-bound linear and cyclic peptide ligands containing the HPQ sequence. Biochemistry 34:15421-9.
Katz, B. A., and R. T. Cass. 1997. In crystals of complexes of streptavidin with peptide ligands containing the HPQ sequence the pKa of the peptide histidine is less than 3.0. J Biol Chem 272:13220-8.
Kedzierski, L., J. Montgomery, D. Bullen, J. Curtis, E. Gardiner, A. Jimenez-Ruiz, and E. Handman. 2004. A leucine-rich repeat motif of Leishmania parasite surface antigen 2 binds to macrophages through the complement receptor 3. J Immunol 172:4902-6.
Koide, A., C. W. Bailey, X. Huang, and S. Koide. 1998. The fibronectin type III domain as a scaffold for novel binding proteins. J Mol Biol 284:1141-51.
Kim, J. S. and Raines, R. T. (1993) Ribonuclease S-peptide as a carrier in fusion proteins. Protein Sci, 2, 348-356.
Lamla, T., and V. A. Erdmann. 2003. Searching sequence space for high-affinity binding peptides using ribosome display. J Mol Biol 329:381-8.
Lang, V., J. Janzen, G. Z. Fischer, Y. Soneji, S. Beinke, A. Salmeron, H. Allen, R. T. Hay, Y. Ben-Neriah, and S. C. Ley. 2003. betaTrCP-mediated proteolysis of NF-kappaB1 p105 requires phosphorylation of p105 serines 927 and 932. Mol Cell Biol 23:402-13.
Larocca, D., A. Witte, W. Johnson, G. F. Pierce, and A. Baird. 1998. Targeting bacteriophage to mammalian cell surface receptors for gene delivery. Hum Gene Ther 9:2393-9.
Larocca, D., and A. Baird. 2001. Receptor-mediated gene transfer by phage-display vectors: applications in functional genomics and gene therapy. Drug Discov Today 6:793-801.
Larocca, D., K. Jensen-Pergakes, M. A. Burg, and A. Baird. 2001. Receptor-targeted gene delivery using multivalent phagemid particles. Mol Ther 3:476-84.
Larocca, D., K. Jensen-Pergakes, M. A. Burg, and A. Baird. 2002. Gene transfer using targeted filamentous bacteriophage. Methods Mol Biol 185:393-401.
Larocca, D., M. A. Burg, K. Jensen-Pergakes, E. P. Ravey, A. M. Gonzalez, and A. Baird. 2002. Evolving phage vectors for cell targeted gene delivery. Curr Pharm Biotechnol 3:45-57.
Larocca, D., P. D. Kassner, A. Witte, R. C. Ladner, G. F. Pierce, and A. Baird. 1999. Gene transfer to mammalian cells using genetically targeted filamentous bacteriophage. Faseb J 13:727-34.
Lauber, T., U. C. Marx, A. Schulz, P. Kreutzmann, P. Rosch, and S. Hoffmann. 2001. Accurate disulfide formation in Escherichia coli: overexpression and characterization of the first domain (HF6478) of the multiple Kazal-type inhibitor LEKTI. Protein Expr Purif 22:108-12.
Lawson, T. G., D. L. Gronros, P. E. Evans, M. C. Bastien, K. M. Michalewich, J. K. Clark, J. H. Edmonds, K. H. Graber, J. A. Werner, B. A. Lurvey, and J. M. Cate. 1999. Identification and characterization of a protein destruction signal in the encephalomyocarditis virus 3C protease. J Biol Chem 274:9871-80.
Le Gall F, Reusch U, Little M, Kipriyanov S M. 2004. Effect of linker sequences between the antibody variable domains on the formation, stability and biological activity of a bispecific tandem diabody. Protein Eng Des Sel 17:357-366.
Legler, D. F., C. Johnson-Leger, G. Wiedle, C. Bron, and B. A. Imhof. 2004. The alpha v beta 3 integrin as a tumor homing ligand for lymphocytes. Eur J Immunol 34:1608-16.
Lesley, S. A., and D. J. Groskreutz. 1997. Simple affinity purification of antibodies using in vivo biotinylation of a fusion protein. J Immunol Methods 207:147-55.
Lety, M. A., C. Frehel, I. Dubail, J. L. Beretti, S. Kayal, P. Berche, and A. Charbit. 2001. Identification of a PEST-like motif in listeriolysin O required for phagosomal escape and for virulence in Listeria monocytogenes. Mol Microbiol 39:1124-39.
Lety, M. A., C. Frehel, P. Berche, and A. Charbit. 2002. Critical role of the N-terminal residues of listeriolysin O in phagosomal escape and virulence of Listeria monocytogenes. Mol Microbiol 46:367-79.
Levin, B. R. and Bull, J. J. (2004) Population and evolutionary dynamics of phage therapy. Nat Rev Microbiol, 2, 166-173.
Leyton, L., P. Schneider, C. V. Labra, C. Ruegg, C. A. Hetz, A. F. Quest, and C. Bron. 2001. Thy-1 binds to integrin beta(3) on astrocytes and triggers formation of focal contact sites. Curr Biol 11: 1028-38.
Liu C., et al. 1996. F(c)gammaRI-targeted fusion proteins result in efficient presentation by human monocytes of antigenic and antagonist T cell epitopes. J. Clin Invest 98:2001-2007.
Lore, K., M. R. Betts, J. M. Brenchley, J. Kuruppu, S. Khojasteh, S. Perfetto, M. Roederer, R. A. Seder, and R. A. Koup. 2003. Toll-like receptor ligands modulate dendritic cells to augment cytomegalovirus- and HIV-1-specific T cell responses. J Immunol 171:4320-8.
Losick, V. P., P. E. Schlax, R. A. Emmons, and T. G. Lawson. 2003. Signals in hepatitis A virus P3 region proteins recognized by the ubiquitin-mediated proteolytic system. Virology 309:306-19.
Mady, B. J., D. V. Erbe, I. Kurane, M. W. Fanger, and F. A. Ennis. 1991. Antibody-dependent enhancement of dengue virus infection mediated by bispecific antibodies against cell surface molecules other than Fc gamma receptors. J Immunol 147:3139-44.
Malik, P. and Perham, R. N. (1996) New vectors for peptide display on the surface of filamentous bacteriophage. Gene, 171, 49-51.
Malik, P. and Perham, R. N. (1997) Simultaneous display of different peptides on the surface of filamentous bacteriophage. Nucleic Acids Res, 25, 915-916.
March, J. B., J. R. Clark, and C. D. Jepson. 2004. Genetic immunisation against hepatitis B using whole bacteriophage lambda particles. Vaccine 22:1666-71.
Marcinkiewicz, C., S. Vijay-Kumar, M. A. McLane, and S. Niewiarowski. 1997. Significance of RGD loop and C-terminal domain of echistatin for recognition of alphaIIb beta3 and alpha(v) beta3 integrins and expression of ligand-induced binding site. Blood 90:1565-75.
Maruyama, I. N., Maruyama, H. I. and Brenner, S. (1994) Lambda foo: a lambda phage vector for the expression of foreign proteins. Proc Natl Acad Sci USA, 91, 8273-8277.
Mathew, E., G. E. Hardee, C. F. Bennett, and K. D. Lee. 2003. Cytosolic delivery of antisense oligonucleotides by listeriolysin O-containing liposomes. Gene Ther 10:1105-15.
McCafferty, J., Griffiths, A. D., Winter, G. and Chiswell, D. J. (1990) Phage antibodies: filamentous phage displaying antibody variable domains. Nature, 348, 552-554.
Meola, A., Delmastro, P., Monaci, P., Luzzago, A., Nicosia, A., Felici, F., Cortese, R. and Galfre, G. (1995) Derivation of vaccines from mimotopes. Immunologic properties of human hepatitis B virus surface antigen mimotopes displayed on filamentous phage. J Immunol, 154, 3162-3172.
Merril, C. R., B. Biswas, R. Carlton, N. C. Jensen, G. J. Creed, S. Zullo, and S. Adhya. 1996. Long-circulating bacteriophage as antibacterial agents. Proc Natl Acad Sci USA 93:3188-92.
Mikawa, Y. G., I. N. Maruyama, and S. Brenner. 1996. Surface display of proteins on bacteriophage lambda heads. J Mol Biol 262:21-30.
Minenkova, O., Pucci, A., Pavoni, E., De Tomassi, A., Fortugno, P., Gargano, N., Cianfriglia, M., Barca, S., De Placido, S., Martignetti, A. et al. (2003) Identification of tumor-associated antigens by screening phage-displayed human cDNA libraries with sera from tumor patients. Int J Cancer, 106, 534-544.
Monaci, P., Urbanelli, L. and Fontana, L. (2001) Phage as gene delivery vectors. Curr Opin Mol Ther, 3, 159-169.
Moore, A. C., W. P. Kong, B. K. Chakrabarti, and G. J. Nabel. 2002. Effects of antigen and genetic adjuvants on immune responses to human immunodeficiency virus DNA vaccines in mice. J Virol 76:243-50.
Morris, M. C., J. Depollier, J. Mery, F. Heitz, and G. Divita. 2001. A peptide carrier for the delivery of biologically active proteins into mammalian cells. Nat Biotechnol 19:1173-6.
Murialdo, H. and Ray, P. N. (1975) Model for arrangement of minor structural proteins in head of bacteriophage lambda. Nature, 257, 815-817.
Nijkamp, H. J., de Lang, R., Stuitje, A. R., van den Elzen, P. J., Veltkamp, E. and van Putten, A. J. (1986) The complete nucleotide sequence of the bacteriocinogenic plasmid CloDF13. Plasmid, 16, 135-160.
Nilsson, B., T. Moks, B. Jansson, L. Abrahmsen, A. Elmblad, E. Holmgren, C. Henrichson, T. A. Jones, and M. Uhlen. 1987. A synthetic IgG-binding domain based on staphylococcal protein A. Protein Eng 1:107-13.
Nomura R, Kiyota A, Suzaki E, Kataoka K, Ohe Y, Miyamoto K, Senda T, Fujimoto T. 2004. Human coronavirus 229E binds to CD13 in rafts and enters the cell through caveolae. J Virol 78:8701-8708.
Ohno, K., K. Sawai, Y. Iijima, B. Levin, and D. Meruelo. 1997. Cell-specific targeting of Sindbis virus vectors displaying IgG-binding domains of protein A. Nat Biotechnol 15:763-7.
Olsson, A., M. Eliasson, B. Guss, B. Nilsson, U. Hellman, M. Lindberg, and M. Uhlen. 1987. Structure and evolution of the repetitive gene encoding streptococcal protein G. Eur J Biochem 168:319-24.
Park, J., J. Ryu, K. A. Kim, H. J. Lee, J. H. Bahn, K. Han, E. Y. Choi, K. S. Lee, H. Y. Kwon, and S. Y. Choi. 2002. Mutational analysis of a human immunodeficiency virus type 1 Tat protein transduction domain which is required for delivery of an exogenous protein into mammalian cells. J Gen Virol 83:1173-81.
Pasqualini R, Koivunen E, Kain R, Lahdenranta J, Sakamoto M, Stryhn A, Ashmun R A, Shapiro L H, Arap W, Ruoslahti E. 2000. Aminopeptidase N is a receptor for tumor-homing peptides and a target for inhibiting angiogenesis. Cancer Res 60:722-727.
Pasqualini, R. and Ruoslahti, E. (1996) Organ targeting in vivo using phage display peptide libraries. Nature, 380, 364-366.
Pavoni, E., Vaccaro, P., Pucci, A., Monteriu, G., Beghetto, E., Barca, S., Dupuis, M. L., De Pasquale Ceratti, A., Lugini, A., Cianfriglia, M. et al. (2004) Identification of a panel of tumor-associated antigens from breast carcinoma cell lines, solid tumors and testis cDNA libraries displayed on lambda phage. BMC Cancer, 4, 78.
Pelkmans L, Kartenbeck J, Helenius A. 2001. Caveolar endocytosis of simian virus 40 reveals a new two-step vesicular-transport pathway to the ER. Nat Cell Biol 3:473-483.
Piersanti, S., Cherubini, G., Martina, Y., Salone, B., Avitabile, D., Grosso, F., Cundari, E., Di Zenzo, G. and Saggio, I. (2004) Mammalian cell transduction and internalization properties of lambda phages displaying the full-length adenoviral penton base or its central domain. J Mol Med, 82, 467-476.
Plank, C., B. Oberhauser, K. Mechtler, C. Koch, and E. Wagner. 1994. The influence of endosome-disruptive peptides on gene transfer using synthetic virus-like gene transfer systems. J Biol Chem 269:12918-24.
Plebanski, M., S. C. Gilbert, J. Schneider, C. M. Hannan, G. Layton, T. Blanchard, M. Becker, G. Smith, G. Butcher, R. E. Sinden, and A. V. Hill. 1998. Protection from Plasmodium berghei infection by priming and boosting T cells to a single class I-restricted epitope with recombinant carriers suitable for human use. Eur J Immunol 28:4345-55.
Pohlmann, S. et al. 2003. Hepatitis C virus glycoproteins interact with DC-SIGN and DC-SIGNR. J Virol 77:4070-4080.
Poul, M. A. and Marks, J. D. (1999) Targeted gene delivery to mammalian cells by filamentous bacteriophage. J Mol Biol, 288, 203-211.
Poul, M. A., Becerril, B., Nielsen, U. B., Morisson, P. and Marks, J. D. (2000) Selection of tumor-specific internalizing human antibodies from phage libraries. J Mol Biol, 301, 1149-1161.
Prchla, E., C. Plank, E. Wagner, D. Blaas, and R. Fuchs. 1995. Virus-mediated release of endosomal content in vitro: different behavior of adenovirus and rhinovirus serotype 2. J Cell Biol 131:111-23.
Proba, K., L. Ge, and A. Pluckthun. 1995. Functional antibody single-chain fragments from the cytoplasm of Escherichia coli: influence of thioredoxin reductase (TrxB). Gene 159:203-7.
Reynaud, A., L. Cloastre, J. Bernard, H. Layeran, H. W. Ackermann, D. Licois, and B. Joly. 1992. Characteristics and diffusion in the rabbit of a phage for Escherichia coli 0103. Attempts to use this phage for therapy. Vet Microbiol 30:203-12.
Richards, J., M. Miller, J. Abend, A. Koide, S. Koide, and S. Dewhurst. 2003. Engineered fibronectin type III domain with a RGDWXE sequence binds with enhanced affinity and specificity to human alphavbeta3 integrin. J Mol Biol 326:1475-88.
Rodriguez, F., L. L. An, S. Harkins, J. Zhang, M. Yokoyama, G. Widera, J. T. Fuller, C. Kincaid, I. L. Campbell, and J. L. Whitton. 1998. DNA immunization with minigenes: low frequency of memory cytotoxic T lymphocytes and inefficient antiviral protection are rectified by ubiquitination. J Virol 72:5174-81.
Ros, C., and C. Kempf. 2004. The ubiquitin-proteasome machinery is essential for nuclear translocation of incoming minute virus of mice. Virology 324:350-60.
Rosenberger, C. M., and B. B. Finlay. 2003. Phagocyte sabotage: disruption of macrophage signalling by bacterial pathogens. Nat Rev Mol Cell Biol 4:385-96.
Salmeron, A., J. Janzen, Y. Soneji, N. Bump, J. Kamens, H. Allen, and S. C. Ley. 2001. Direct phosphorylation of NF-kappaB1 p105 by the IkappaB kinase complex on serine 927 is essential for signal-induced p105 proteolysis. J Biol Chem 276:22215-22.
Santala, V., and U. Lamminmaki. 2004. Production of a biotinylated single-chain antibody fragment in the cytoplasm of Escherichia coli. J Immunol Methods 284:165-75.
Santi, E., Capone, S., Mennuni, C., Lahm, A., Tramontano, A., Luzzago, A. and Nicosia, A. (2000) Bacteriophage lambda display of complex cDNA libraries: a new approach to functional genomics. J Mol Biol, 296, 497-508.
Santini, V., B. Scappini, Z. K. Indik, A. Gozzini, P. R. Ferrini, and A. D. Schreiber. 2003. The carboxy-terminal region of the granulocyte colony-stimulating factor receptor transduces a phagocytic signal. Blood 101:4615-22. Epub 2003 Feb. 13.
Sauder, D. N., M. H. Smith, T. Senta-McMillian, I. Soria, and T. C. Meng. 2003. Randomized, single-blind, placebo-controlled study of topical application of the immune response modulator resiquimod in healthy adults. Antimicrob Agents Chemother 47:3846-52.
Scarborough, R. M., J. W. Rose, M. A. Naughton, D. R. Phillips, L. Nannizzi, A. Arfsten, A. M. Campbell, and I. F. Charo. 1993. Characterization of the integrin specificities of disintegrins isolated from American pit viper venoms. J Biol Chem 268:1058-65.
Scheibler, L., D. F. Mierke, G. Bitan, M. Rosenblatt, and M. Chorev. 2001. Identification of a contact domain between echistatin and the integrin alpha(v)beta(3) by photoaffinity cross-linking. Biochemistry 40:15117-26.
Schneider, J., S. C. Gilbert, C. M. Hannan, P. Degano, E. Prieur, E. G. Sheu, M. Plebanski, and A. V. Hill. 1999. Induction of CD8+ T cells using heterologous prime-boost immunisation strategies. Immunol Rev 170:29-38.
Schoolnik, G. K., W. C. Summers, and J. D. Watson. 2004. Phage offer a real alternative. Nature Biotechnology 22:505-6.
Schwarze, S. R., A. Ho, A. Vocero-Akbani, and S. F. Dowdy. 1999. In vivo protein transduction: delivery of a biologically active protein into the mouse. Science 285:1569-72.
Skehel, J. J., K. Cross, D. Steinhauer, and D. C. Wiley. 2001. Influenza fusion peptides. Biochem Soc Trans 29:623-6.
Smith, G. P. (1985) Filamentous fusion phage: novel expression vectors that display cloned antigens on the virion surface. Science, 228, 1315-1317.
Smith, G. P. (1993) Surface display and peptide libraries. Gene, 128, 1-2.
Smith, G. P. and Scott, J. K. (1993) Libraries of peptides and proteins displayed on filamentous phage. Methods Enzymol, 217, 228-257.
Smith, M. P. and Feiss, M. (1993) Sites and gene products involved in lambdoid phage DNA packaging. J Bacteriol, 175, 2393-2399.
Sternberg, N. and Weisberg, R. (1977) Packaging of coliphage lambda DNA. II. The role of the gene D protein. J Mol Biol, 117, 733-759.
Sternberg, N., and R. H. Hoess. 1995. Display of peptides and proteins on the surface of bacteriophage lambda. Proc Natl Acad Sci USA 92:1609-13.
Sudowe S, Ludwig-Portugall I, Montermann E, Ross R, Reske-Kunz A B. 2003. Transcriptional targeting of dendritic cells in gene gun-mediated DNA immunization favors the induction of type 1 immune responses. Mol Ther 8:567-575.
Suhrbier, A., and M. La Linn. 2003. Suppression of antiviral responses by antibody-dependent enhancement of macrophage infection. Trends Immunol 24:165-8.
Takada, A., and Y. Kawaoka. 2003. Antibody-dependent enhancement of viral infection: molecular mechanisms and in vivo implications. Rev Med Virol 13:387-98.
Takkinen K, Laukkanen M L, Sizmann D, Alfthan K, Immonen T, Vanne L, Kaartinen M, Knowles J K, Teeri T T. 1991. An active single-chain antibody containing a cellulase linker domain is secreted by Escherichia coli. Prot Eng 4:837-841.
Thibault, G. 2000. Sodium dodecyl sulfate-stable complexes of echistatin and RGD-dependent integrins: a novel approach to study integrins. Mol Pharmacol 58:1137-45.
Tselepis, V. H., L. J. Green, and M. J. Humphries. 1997. An RGD to LDV motif conversion within the disintegrin kistrin generates an integrin antagonist that retains potency but exhibits altered receptor specificity. Evidence for a functional equivalence of acidic integrin-binding motifs. J Biol Chem 272:21341-8.
Urbanelli, L., C. Ronchini, L. Fontana, S. Menard, R. Orlandi, and P. Monaci. 2001. Targeted gene transduction of mammalian cells expressing the HER2/neu receptor by filamentous phage. J Mol Biol 313:965-76.
Utaisincharoen, P., W. Kespichayawattana, N. Anuntagool, P. Chaisuriya, S. Pichyangkul, A. M. Krieg, and S. Sirisinha. 2003. CpG ODN enhances uptake of bacteria by mouse macrophages. Clin Exp Immunol 132:70-5.
Valladeau, J. et al. 2000. Langerin, a novel C-type lectin specific to Langerhans cells, is an endocytic receptor that induces the formation of Birbeck granules. Immunity 12:71-81.
Venturi, M., C. Seifert, and C. Hunte. 2002. High level production of functional antibody Fab fragments in an oxidizing bacterial cytoplasm. J Mol Biol 315:1-8.
Volpers, C., C. Thirion, V. Biermann, S. Hussmann, H. Kewes, P. Dunant, H. von der Mark, A. Herrmann, S. Kochanek, and H. Lochmuller. 2003. Antibody-mediated targeting of an adenovirus vector modified to contain a synthetic immunoglobulin g-binding domain in the capsid. J Virol 77:2093-104.
Wadia, J. S., R. V. Stan, and S. F. Dowdy. 2004. Transducible TAT-HA fusogenic peptide enhances escape of TAT-fusion proteins after lipid raft macropinocytosis. Nat Med 10:310-5. Epub 2004 Feb. 8.
Weber-Dabrowska, B., M. Dabrowski, and S. Slopek. 1987. Studies on bacteriophage penetration in patients subjected to phage therapy. Arch Immunol Ther Exp (Warsz) 35:563-8.
Weiss, J. M., A. C. Renkl, C. S. Maier, M. Kimmig, L. Liaw, T. Ahrens, S. Kon, M. Maeda, H. Hotta, T. Uede, and J. C. Simon. 2001. Osteopontin is involved in the initiation of cutaneous contact hypersensitivity by inducing Langerhans and dendritic cell migration to lymph nodes. J Exp Med 194:1219-29.
West, M. A., R. P. Wallin, S. P. Matthews, H. G. Svensson, R. Zaru, H. G. Ljunggren, A. R. Prescott, and C. Watts. 2004. Enhanced dendritic cell antigen capture via toll-like receptor-induced actin remodeling. Science 305:1153-7.
Wiedle, G., C. Johnson-Leger, and B. A. Imhof. 1999. A chimeric cell adhesion molecule mediates homing of lymphocytes to vascularized tumors. Cancer Res 59:5255-63.
Wittamer, V., F. Gregoire, P. Robberecht, G. Vassart, D. Communi, and M. Parmentier. 2004. The C-terminal nonapeptide of mature chemerin activates the chemerin receptor with low nanomolar potency. J Biol Chem 279:9956-62.
Wittamer, V., J. D. Franssen, M. Vulcano, J. F. Miijolet, E. Le Poul, I. Migeotte, S. Brezillon, R. Tyldesley, C. Blanpain, M. Detheux, A. Mantovani, S. Sozzani, G. Vassart, M. Parmentier, and D. Communi. 2003. Specific recruitment of antigen-presenting cells by chemerin, a novel processed ligand from human inflammatory fluids. J Exp Med 198:977-85.
Woof, J. N. 2002. The human IgA-Fc alpha receptor interaction and its blockade by streptococcal IgA-binding proteins. Biochem Soc Trans 30:491-494.
Yang, F., P. Forrer, Z. Dauter, J. F. Conway, N. Cheng, M. E. Cerritelli, A. C. Steven, A. Pluckthun, and A. Wlodawer. 2000. Novel fold and capsid-binding properties of the lambda-phage display platform protein gpD. Nat Struct Biol 7:230-7.
Yen L, Svendsen J, Lee J S, Gray J T, Magnier M, Baba T, D'Amato R J, Mulligan R C. 2004. Exogenous control of mammalian gene expression through modulation of RNA self-cleavage. Nature 431:471-476.
Yu, J. and Smith, G. P. (1996) Affinity maturation of phage-displayed peptide ligands. Methods Enzymol, 267, 3-27.
Zhong, G., Smith, G. P., Berry, J. and Brunham, R. C. (1994) Conformational mimicry of a chlamydial neutralization epitope on filamentous phage. J Biol Chem, 269, 24183-24188.

1. An antigen delivery system comprising a modified lambda phage and a phage encoded antigen.

2. The antigen delivery system of claim 1, wherein the lambda phage comprises a surface polypeptide modified to target an antigen-presenting cell.

3. The antigen delivery system of claim 2, wherein the surface polypeptide is gpD.

4. The antigen delivery system of claim 3, wherein the surface polypeptide comprises the amino acid sequence of SEQ ID NO:39.

5. The antigen delivery system of claim 3, wherein the surface polypeptide is encoded by the nucleic acid sequence of SEQ ID NO:52.

6. The antigen delivery system of claim 2, wherein the surface polypeptide is gpv.

7. The antigen delivery system of claim 6, wherein the surface polypeptide comprises the amino acid sequence of SEQ ID NO:40.

8. The antigen delivery system of claim 6, wherein the surface polypeptide is encoded by the nucleic acid sequence of SEQ ID NO:53.

9. The antigen delivery system of claim 2, wherein, the surface polypeptide is D-protein homologue of a non-lambda phage.

10. The antigen delivery system of claim 2, wherein the surface polypeptide is a codon-optimized derivative of gpD.

11. The antigen delivery system of claim 10, wherein the surface polypeptide is encoded by the nucleic acid sequence of SEQ ID NO:1.

12. The antigen delivery system of claim 2, wherein the modified surface polypeptide comprises a polypeptide that binds a chemokine receptor.

13. The antigen delivery system of claim 12, wherein the chemokine receptor-binding polypeptide is chemerin or a chemerin-like peptide that binds the chemerin receptor.

14. The antigen delivery system of claim 2, wherein the modified surface polypeptide comprises a polypeptide that binds an integrin.

15. The antigen delivery system of claim 14, wherein the integrin binding polypeptide is FnfnlO-3JCL14.

16. The antigen delivery system of claim 14, wherein the integrin binding polypeptide is a integrin-binding snake venom polypeptide or an integrin-binding variant or fragment of the snake venom polypeptide.

17. The antigen delivery system of claim 16, wherein the snake venom polypeptide is a disintegrin or a derivative of the disintegrin.

18. The antigen delivery system of claim 17, wherein the polypeptide is derived from a snake-venom disintegrin selected from a group consisting of flavoridin, lachesin, echistatin, and kistrin.

19. The antigen delivery system of claim 2, wherein the modified surface further comprises a basic peptide transduction domain (PTD).

20. The antigen delivery system of claim 19, wherein the PTD is derived from HIV-I Tat.

21. The antigen delivery system of claim 2, wherein the surface polypeptide is modified to comprise a biotin moiety.

22. The antigen delivery system of claim 21, further comprising an avidin-linked polypeptide that targets an endocytosing receptor on the antigen-presenting cell.

23. The antigen delivery system of claim 22, wherein the avidin-linked polypeptide is transferrin, mannose, or a derivative of transferrin or mannose that blinds an endocytosing receptor.

24. The antigen delivery system of claim 22, wherein the avidin-linked polypeptide is an antibody or fragment thereof.

25. The antigen delivery system of claim 23, wherein the antibody or fragment binds DEC205, DC-SIGN, or CD64/FcγR1.

26. The antigen delivery system of claim 21, wherein the modified surface polypeptide further comprises a basic peptide transduction domain.

27. The antigen delivery system of claim 2, wherein the surface polypeptide comprises an IgG Fc-binding motif.

28. The antigen delivery system of claim 27, wherein the IgG Fc-binding motif comprises a viral IgG-binding polypeptide.

29. (canceled)

30. The antigen delivery system of claim 27, further comprising an IgG antibody or fragment thereof, wherein the IgG antibody or fragment binds DEC205, DC-SIGN, or CD64/FcγR1.

31. The antigen delivery system of claim 27, wherein the modified surface further comprises a basic peptide transduction domain.

32. The antigen delivery system of claim 2, wherein the surface polypeptide is modified to incorporate an IgA-binding motif.

33. The antigen delivery system of 32, wherein the IgA-binding motif comprises a bacterial IgA-binding polypeptide.

34. The antigen delivery system of 32, further comprising an IgA antibody or fragment thereof, wherein the IgA antibody or fragment binds DEC205, DC-SIGN, or CD64/Fc-γR1.

35. The antigen delivery system of 32, wherein the modified surface further comprises a basic peptide transduction domain.

36-38. (canceled)

39. The antigen delivery system of claim 1, wherein the lambda phage comprises more than one surface polypeptide and wherein at least one of the surface polypeptides is modified to target an antigen-presenting cell.

40. The antigen delivery system of claim 39, wherein at least one surface polypeptide is unmodified.

41. (canceled)

42. The antigen delivery system of claim 39, wherein the surface polypeptide is gpD.

43. The antigen delivery system of claim 39, wherein the surface polypeptide is gpv.

44. The antigen delivery system of claim 39, wherein the surface polypeptide is a D-protein homologue of a non-lambda phage.

45. The antigen delivery system of claim 39, wherein the surface polypeptide is a codon-optimized derivative of gpD.

46. The antigen delivery system of claim 39, wherein the modified surface polypeptide comprises a polypeptide that binds a chemokine receptor.

47. The antigen delivery system of claim 39, wherein the modified surface polypeptide comprises a polypeptide that binds an integrin.

48. The antigen delivery system of claim 39, wherein the modified surface polypeptide further comprises a basic peptide transduction domain (PTD).

49. The antigen delivery system of claim 39, wherein the surface polypeptide is modified to comprise a biotin moiety.

50. The antigen delivery system of claim 39, wherein the surface polypeptide comprises an IgG Fc-binding motif.

51. The antigen delivery system of claim 39, wherein the surface polypeptide is modified to incorporate an IgA-binding motif.

52. The antigen delivery system of claim 39, wherein the modified surface polypeptide comprises an antibody fusion proteins, wherein the antibody fusion protein comprises a single chain antibody to an endocytosing receptor.

53. A nucleic acid that encodes a surface polypeptide modified to target an antigen-presenting cell.

54. The nucleic acid of claim 53, wherein the surface polypeptide is gpD.

55. The nucleic acid of claim 53, wherein the surface polypeptide is gpV.

56. The nucleic acid of claim 53, wherein the surface polypeptide is a homologous D-protein.

57. The nucleic acid of claim 53, further comprising a nucleotide sequence that encodes a basic peptide transduction domain.

58. A vector comprising the nucleic acid of claim 53, operably linked to an expression control sequence.

59. A vector comprising the nucleic acid of claim 57, operably linked to an expression control sequence.

60. The antigens delivery system of claim 1, wherein the lambda phage comprises a mutation in a coat protein.

61. The antigen delivery system of claim 60, wherein the mutated coat protein is a lambda E capsid protein.

62. The antigen delivery system of claim 61, wherein the lambda E capsid protein comprises an F to K mutation at residue 158.

63. The antigen delivery system of claim 40, wherein the lambda phage further comprises a modified surface polypeptide and wherein the modified surface polypeptide comprises a basic peptide transduction domain.

64. The antigen delivery system of claim 1, wherein the lambda phage comprises an antibody-surface polypeptide conjugate.

65. The antigen delivery system of claim 1, wherein the lambda phage comprises a surface polypeptide modified to enhance endosomal escape.

66. The antigen delivery system of claim 65, wherein the modified surface polypeptide, comprises a HA2 polypeptide, a GALA-INF3 polypeptide, or a peptide derived from LLO (listeriolysin).

67. The antigen delivery system of claim 65, wherein the modified surface polypeptide further comprises a basic peptide transduction domain.

68. A modified lambda phage comprising a surface polypeptide fusion protein, wherein the fusion protein destabilizes a viral capsid.

69. The modified lambda phage of claim 68, wherein the polypeptide of the fusion protein comprises a PEST motif.

70. The modified lambda phage of claim 68, wherein the polypeptide of the fusion protein comprises a ubiquitination motif.

71. The modified lambda phage of claim 69, wherein the fusion protein further comprises a basic peptide transduction domain.

72. The modified lambda phage of claim 70, wherein the modified surface further comprises a basic peptide transduction domain.

73. A nucleic acid that encodes a surface polypeptide fusion protein, wherein the fusion protein destabilizes a viral capsid.

74. A vector comprising the nucleic acid of claim 73 operably linked to an expression control sequence.

75. The antigen delivery system of claim 1, wherein the surface modified to promote non-phagocytic/non-endosomal membrane transport.

76. The antigen delivery system of claim 75, wherein the modified surface polypeptide comprises a polypeptide selected from the group consisting of a peptide transduction domain, an E. coli pilus protein, or Leishmania PSA-2.

77. A method of promoting an antigenic response in a subject, comprising administering to the subject the antigen delivery system of claim 2.

78. The method of claim 77, further comprising administering to the subject a granulocyte colony stimulating factor.

79. The method of claim 77, further comprising administering to the subject a Toll-like receptor ligand.

80. The method of claim 77, further comprising administering to the subject a PTD-derived polypeptide.

81. The method of claim 77, further comprising administering to the subject a viral vector comprising the immunogen.

82. (canceled)

83. A method of making a lambda phage with a modified surface polypeptide, comprising the steps of:
a. Inserting into a surface polypeptide encoding nucleic acid more than one nucleotide sequence that encodes exogeneous polypeptide sequences; and
b. Expressing the surface polypeptide and exogenous polypeptide sequences in the lambda phage.

84. The method of claim 83, wherein the exogeneous polypeptide sequences are encoded at both the N and C terminus of the modified surface polypeptide.

85. The method of claim 83, wherein the exogeneous polypeptide sequences include flexible linker polypeptides.

86. A method of making a lambda phage with a modified surface polypeptide, comprising the steps of:
a. transforming a surface polypeptide-deficient lambda lysogen with a plurality of nucleic acids, wherein the nucleic acids encode a plurality of gpD proteins, wherein at least one nucleic acid encodes a modified gpD protein and wherein the surface polypeptides comprise exogenous polypeptides; and
b. expressing the surface polypeptide and exogenous polypeptide sequences in the lambda phage.

87. The method of claim 86, wherein the transformation step is performed using a plurality of plasmids encoding various surface polypeptides.

88. The method of claim 86, wherein the transformation step is performed using a plasmid encoding all of the surface polypeptides.

89. A method of eliciting an antigenic response in a subject, comprising administering to the subject a modified lambda phage.

90. A gene delivery system comprising a modified lambda phage and a phage encoded gene of interest.

91. The gene delivery system of claim 90, wherein the lambda phage comprises a surface polypeptide modified to target a selected cell.

92. The gene delivery system of claim 90, wherein the lambda phage comprises more than one surface polypeptide and wherein at least one of the surface polypeptides is modified to target a selected cell.


SEQUENCE LISTING (66 sequences)

SEQ ID NO: 1 - DNA, 333 bp, Artificial Sequence
  Description of Artificial Sequence; note = synethetic construct
atgaccagcaaggaaacatttacgcattaccagccgcaagggaactctgatcccgcgcacaccgccaccgccccgggcggactgtcagcgaaggccccggccatgactcctctgatgttagatacgagcagtcggaaacttgtggcgtgggacggtactaccgatggtgcagcggtcggcattttggcagttgcagctgaccagacatcgaccactctgacattctataaatccggcacctttcgttatgaggatgtgctctggccagaagccgcttctgacgaaacgaaaaaacgcacggcgttcgcgggcaccgcaatcagtattgtatag

SEQ ID NO: 2 - protein, 9 aa, Artificial Sequence
  Description of Artificial Sequence; note = synethetic construct
YFPGQFAFS

SEQ ID NO: 3 - protein, 10 aa, Artificial Sequence
  Description of Artificial Sequence; note = synethetic construct
YFLPGPFAFS

SEQ ID NO: 4 - protein, 96 aa, Artificial Sequence
  Description of Artificial Sequence; note = synethetic construct
MQVSDVPRDLEVVAATPTSLLISWDAPAVTVRYYRITYGETGGNSPVQEFTVPGSKSTATISGLKPGVDYTITVYAVTGRGDWNEGSKPISINYRT

SEQ ID NO: 5 - protein, 70 aa, Artificial Sequence
  Description of Artificial Sequence; note = synethetic construct
GEECDCGSPSNPCCDAATCKLRPGAQCADGLCCDQCRFKKKRTICRIARGDFPDDRCTGLSNDCPRWNDL

SEQ ID NO: 6 - DNA, 223 bp, Artificial Sequence
  Description of Artificial Sequence; note = synethetic construct
ggtaccggcgaagaatgcgattgcggcagcccgagcaacccgtgctgcgatgcggcgacctgcaaactgcgtccgggtgcgcagtgcgcggatggcctgtgctgcgatcagtgccgttttaagaaaaaacgtaccatttgccgtattgcgcgtggcgattttccggatgatcgttgcaccggcctgagcaacgattgcccgcgttggaacgatctgtaagctt

SEQ ID NO: 7 - protein, 73 aa, Artificial Sequence
  Description of Artificial Sequence; note = synethetic construct
EAGEECDCGAPANPCCDAATCKLRPGAQCAEGLCCDQCRFIKKGKICRRARGDNPDDRCTGQSADCPRNGYYG

SEQ ID NO: 8 - DNA, 232 bp, Artificial Sequence
  Description of Artificial Sequence; note = synethetic construct
ggtaccgaagcgggcgaagaatgcgattgcggcgcaccggcgaacccgtgctgcgatgcggcgacctgcaaactgcgtccgggtgcgcagtgcgcggaaggcctgtgctgcgatcagtgccgttttatcaaaaaaggcaaaatttgccgtcgtgcgcgtggcgataacccggatgatcgttgcaccggccagagcgcggattgcccgcgtaacggctattatggctaagctt

SEQ ID NO: 9 - protein, 13 aa, Artificial Sequence
  Description of Artificial Sequence; note = synethetic construct
CRIPRGDMPDDRC

SEQ ID NO: 10 - protein, 9 aa, Artificial Sequence
  Description of Artificial Sequence; note = synethetic construct
DVEAWLGAR

SEQ ID NO: 11 - protein, 14 aa, Artificial Sequence
  Description of Artificial Sequence; note = synethetic construct
AEFSHPQNTIEGRK

SEQ ID NO: 12 - protein, 15 aa, Artificial Sequence
  Description of Artificial Sequence; note = synethetic construct
GGLNDIFEAQKIEWH

SEQ ID NO: 13 - protein, 116 aa, Artificial Sequence
  Description of Artificial Sequence; note = synethetic construct
DNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKV

SEQ ID NO: 14 - DNA, 478 bp, Artificial Sequence
  Description of Artificial Sequence; note = synethetic construct
ccagtgaattcgagctcggtacccggggatcctgccgagctcgtcgacaagcttctatttaggtgcctgcgcatcgttcaacttttttgcctcggctaacagattggcagactgacttgggtcatctttcaggctctgaataaacgcattacgttgttcctcgttaaggttcgggagatgcaggatttcatagaaagcattttgctgttccttattaaatttgttatccactttcggcgcctgagcatcatttagctttttagcttctgctaaaaggttagcgctttggcttgggtcatcttttaaactttggatgaaggcgtttcgttgttcttcgtttaagttaggtaaatgtaagatctcatagaacgcgttttgttgttctttgttgaatttgttgtctacggtaccgaattcctcgaggatatcccatctagagtcgacctgcaggcatgcaagcttggcgtaatcatggtca

SEQ ID NO: 15 - protein, 55 aa, Artificial Sequence
  Description of Artificial Sequence; note = synethetic construct
TYKLVINGKTLKGETTTEAVDAATAEKVFKQYANDNGVDGEWTYDDATKTFTVTA

SEQ ID NO: 16 - protein, 19 aa, Artificial Sequence
  Description of Artificial Sequence; note = synethetic construct
KENSISSMAPPASPPASPK

SEQ ID NO: 17 - protein, 30 aa, Artificial Sequence
  Description of Artificial Sequence; note = synethetic construct
DASAFNKENSISSMAPPASPPASPKTPIEK

SEQ ID NO: 18 - protein, 20 aa, Artificial Sequence
  Description of Artificial Sequence; note = synethetic construct
RSEVTISPAETPESPPATPK

SEQ ID NO: 19 - protein, 18 aa, Artificial Sequence
  Description of Artificial Sequence; note = synethetic construct
LRDSDSVCDSGVETSFRK

SEQ ID NO: 20 - protein, 14 aa, Artificial Sequence
  Description of Artificial Sequence; note = synethetic construct
LLVRGRTLVVMMGS

SEQ ID NO: 21 - protein, 10 aa, Artificial Sequence
  Description of Artificial Sequence; note = synethetic construct
LGVKDDWLLV

SEQ ID NO: 22 - protein, 76 aa, Artificial Sequence
  Description of Artificial Sequence; note = synethetic construct
MQIFVKTLTGKTITLEVEPSDTIENVKAKIQDKEGIPPDQQRLIFAGKQLEDGRTLSDYNIQKESTLHLVLRLRGA

SEQ ID NO: 23 - protein, 20 aa, Artificial Sequence
  Description of Artificial Sequence; note = synethetic construct
GDIMGEWGNEIFGAIAGFLG

SEQ ID NO: 24 - protein, 33 aa, Artificial Sequence
  Description of Artificial Sequence; note = synethetic construct
GLFEAIEGFIENGWEGLAEALAEALEALAAGGS

SEQ ID NO: 25 - protein, 16 aa, Artificial Sequence
  Description of Artificial Sequence; note = synethetic construct
RQIKIWFQNRRMKWKK

SEQ ID NO: 26 - protein, 11 aa, Artificial Sequence
  Description of Artificial Sequence; note = synethetic construct
GRKKRRQRPPQ

SEQ ID NO: 27 - protein, 21 aa, Artificial Sequence
  Description of Artificial Sequence; note = synethetic construct
KETWWETWWTEWSQPKKKRKV

SEQ ID NO: 28 - protein, 300 aa, Artificial Sequence
  Description of Artificial Sequence; note = synethetic construct
MKRVITLFAVLLMGWSVNDWSFACKTANGTAIPIGGGSANVYVNLAPVVNVGQNLVVDLSTQIFCHNDYPETITDYVTLQRGSAYGGVLSNFSGTVKYSGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 29 - DNA, 232 bp, Artificial Sequence; identical to SEQ ID NO: 8
  Description of Artificial Sequence; note = synethetic construct

SEQ ID NO: 30 - protein, 479 aa, Artificial Sequence
  Description of Artificial Sequence; note = synethetic construct
VALLLCTSSAPVARAAGTGDFTDEQRTHTLAVLQAFGRAIPELGKNWTGDDFCSWDHVTCAPLSVAVGLDVSTYAGTLPEIPVGVDYRYVMIRDLGFWNMPLLVGTLPDSWSNLIRLSFLTLSGCGVSGTLPASWSSMAFLRELTVGNCGRLTGSLPPEWGSMPNLSRLVLRQLQLSGTLPAEWSRMRLVLTLEIAAAGSFTGTLPAEWSSIKSLANLYLDDLPIGGSLPPEWGSMANLRELKIWELPLTGPLPPQWGSIAGLRVLSLRGTSISGTLPPEWSSMKFLTNLDLGGANISGSLPPQWGSLEFLRGLDLQGTKVSGTLPPGWSEMKWMTFLYLELTQLSGTLPSEWSSMTRLRDLNLHGANISGSLPPEWTSMAALNFLDLGGTQVSGSLPPQWSTMLSAEILQLEHCDLSGSLPPEWSAMPKLRLVGLKGNHFCGCVPDSWAQNAGLVVRIEDEHKGKYCRLGNDCRTTEP

SEQ ID NO: 31 - DNA, 1453 bp, Artificial Sequence
  Description of Artificial Sequence; note = synethetic construct
ggtaccatggtggcgctgctgctgtgcaccagcagcgcaccggttgcgcgtgcggcaggcaccggcgatttcaccgatgaacagcgtacccataccctggcggtgctgcaggcgtttggccgtgcgattccggaactgggcaaaaactggaccggcgatgatttttgcagctgggatcatgtgacctgtgcgccgctgagcgtggcggtgggcctggatgtgagcacctatgcgggcaccctgccggaaattccggtgggcgtggattatcgttatgtgatgattcgtgatctgggcttttggaacatgccgctgctggttggcacgctgccggatagctggagcaacctgattcgtctgagctttctgaccctgagcggctgcggcgtgtctggcaccctgccggcgagctggagcagcatggcgtttctgcgtgaactgaccgtgggcaactgcggccgtctgaccggtagcctgccgccggaatggggcagcatgccgaacctgagccgtctggtgctgcgtcagctgcagctgagcggtacgctgccggccgaatggagccgtatgcgcctggtgctgaccctggaaattgcggcggcaggcagctttaccggcactctgccagcggaatggtctagcattaaaagcctggcgaacctgtatctggatgatctgccgattggcggctctctgccgccagagtggggctctatggcgaatctgcgtgaactgaaaatttgggaactgccgctgaccggtccgctgccgccgcagtggggcagcattgcgggcctgcgtgtgctgagcctgcgtggcaccagcattagcggcaccctgccgccggaatggtctagcatgaaatttctgaccaacctggatctgggcggtgcgaacatttctggtagcctgccgccacaatggggtagcctggaatttctgcgtggcctggatctgcagggcaccaaagtgagcggtacgctgccaccgggctggagcgaaatgaaatggatgacctttctgtatctggaactgacccagctgtctggcacgctgccgagcgaatggagcagcatgacccgtctgcgcgatctgaacctgcatggcgcgaacattagcggcagcctgccgccggaatggacctctatggcggcgctgaactttctggatctgggcggcacccaggtgagcggctctctgccgccgcagtggagcaccatgctgagcgcggaaattctgcagctggaacattgcgatctgtctggttctctgccaccagaatggagcgcgatgccgaaactgcgtctggtgggcctgaaaggcaaccatttttgcggctgcgtgccggatagctgggcgcagaacgcgggcctggtggtgcgtattgaagatgaacataaaggcaaatattgccgtctgggcaacgattgccgtaccaccgaaccgtaagctt

SEQ ID NO: 32 - DNA, 23 bp, Artificial Sequence
  Description of Artificial Sequence; note = synethetic construct
tcgtcgtttttcggcgcgcgccg

SEQ ID NO: 33 - DNA, 24 bp, Artificial Sequence
  Description of Artificial Sequence; note = synethetic construct
tcgtcgttttgtcgttttgtcgtt

SEQ ID NO: 34 - protein, 16 aa, Artificial Sequence
  Description of Artificial Sequence; note = synethetic construct
RQIKIWFQNRRMKWKK

SEQ ID NO: 35 - protein, 11 aa, Artificial Sequence
  Description of Artificial Sequence; note = synethetic construct
GRKKRRQRPPQ

SEQ ID NO: 36 - protein, 30 aa, Artificial Sequence
  Description of Artificial Sequence; note = synethetic construct
RRRQRRKKRGGDIMGEWGNEIFGAIAGFLG

SEQ ID NO: 37 - protein, 21 aa, Artificial Sequence
  Description of Artificial Sequence; note = synethetic construct
KETWWETWWTEWSQPKKKRKV

SEQ ID NO: 38 - protein, 9 aa, Artificial Sequence
  Description of Artificial Sequence; note = synethetic construct
RKKRRQRRR

SEQ ID NO: 39 - protein, 110 aa, Artificial Sequence
  Description of Artificial Sequence; note = synethetic construct
MTSKETFTHYQPQGNSDPAHTATAPGGLSAKAPAMTPLMLDTSSRKLVAWDGTTDGAAVGILAVAADQTSTTLTFYKSGTFRYEDVLWPEAASDETKKRTAFAGTAISIV

SEQ ID NO: 40 - protein, 246 aa, Artificial Sequence
  Description of Artificial Sequence; note = synethetic construct
MPVPNPTMPVKGAGTTLWVYKGSGDPYANPLSDVDWSRLAKVKDLTPGELTAESYDDSYLDDEDADWTATGQGQKSAGDTSFTLAWMPGEQGQQALLAWFNEGDTRAYKIRFPNGTVDVFRGWVSSIGKAVTAKEVITRTVKVTNVGRPSMAEDRSTVTAATGMTVTPASTSVVKGQSTTLTVAFQPEGVTDKSFRAVSADKTKATVSVSGMTITVNGVAAGKVNIPVVSGNGEFAAVAEITVTAS

SEQ ID NO: 41 - protein, 115 aa, Artificial Sequence
  Description of Artificial Sequence; note = synethetic construct
MVTKTITEQRAEVRIFAGNDPAHTATGSSGISSPTPALTPLMLDEATGKLVVWDGQKAGSAVGILVLPLEGTETALTYYKSGTFATEAIHWPESVDEHKKANAFAGSALSHAALP

SEQ ID NO: 42 - DNA, 8134 bp, Artificial Sequence
  Description of Artificial Sequence; note = synethetic construct
aatatttctgattttcatttgatgatgcctctgtgtgaaatgacggtaaacgacgcacttgtgccggcacataatagcaagcaccataatagatcagattcgattcttgctgtaagtgataattattctcgttttcgggtcctttccgtcgatccaacaggttacggggcggcgacctcgcggtttttcactatttatgaaaatttttcagggaaaatcgtgtcggtacttctcgaatataactttttgttttttttaatattgcatccgtaaaggtccgacatgaaagtgtccgaaaatgcctttttctggcgttttcatgtcgggccttgtatttgataatgggttgttttcatgaaggttaataaaaagaggcttgccgaaattttcaacgtggacccgcggacgattgaacgctggcagtctcagggactcccttgcgcctccaaaggtagtaagggcattgaatctgtatttgatactgccatggcaattcagtggtatgcgcagagggaaactgatatcgaaaacgaaaagctccgcaaagaactggacgatttgcgtgcggcagcggagtcagatttacaacccggcaccattgactatgaacgctaccggctcacaaaagcgcaggcagatgcgcaggaactgaaaaatgcccgtgaagacggagtagtgctggaaactgaactgtttaccttcattctgcaacgtgtggcacaggagatttcggggatacttgtgcgtgtgccgttgacattacagcgtaaatatccggacatttcaccatcacaccttgatgtggtgaaaactgaaatcgcgaaagcctccaatgttgcagctaaggccggtgaaaacgtgggcgggtggatcgatgatttcagacgcgcagaaggcagctaatgcagccggtgcgatagctacagggcttttatctctcattattcctgttccactgacgacagttcagtgggccaataaacattattaccttcctaaagagtcgtcttataccccggggcgatgggaaacactgccgtttcaggttggcatcatgaactgtatgggcaacgatctgattcgcacggttaacctgattaaatctgcccgtgttggttatacaaagatgttgctgggagtggaggcttattttattgagcataaatcacgcaacagccttctttttcagcccacggactcagctgctgaagattttatgaaatctcatgttgagccaacgataagggatgttcctgctatgctggagctggctccatggttcggaagaaaacaccgcgataatacgctcaccctgaagcgtttttcctccggtgtggggttctggtgtctgggtggtgcggcagcaaaaaactaccgtgaaaaatccgtggatgtggtctgttatgacgagctttcctcgttcgaaccggatgttgaaaaagagggttcgccaaccctgctgggggataaacgtattgagggctctgtatggccaaaatccattcgcggctcgacgccaaaaatcaaaggctcctgccagatcgaaaaagccgctaacgagtcggcacacttcatgcgtttttatgtgccctgtccgcactgtggggaggagcagtatctgaaatttggcgatgatgcctcgcctttcggtcttaagtgggagaagaataagccagaaagtgttttctacctttgcgagcatcatggctgtgtgatccatcagtctgagcttgaccagagtaacgggcggtggatctgtgaaaacacgggcatgtggacccgtgacggcctgatgtttttcagcgcccggggtgatgaaattccgccgccgcgctccatcactttccatatctggacggcgtacagtccgttcaccacctgggtacagattgtctatgactggctggatgcactgaaagatcccaacggcctgaaaacctttgtgaacaccacgctgggcgagacctgggaagaggctgtgggcgaaaaactcgatcaccaggtgctgatggataaggttgtgcgttacacggcggcggtgcctgcccgggtggtttatctgacggcgggcattgactcgcagcgaaaccgttttgagatgtatgtctggggatgggctccgggagaggaagcctttctggtggataaaatcatcattatggggcgtcccgatgaggaagagacgctgttacgtgtggatgcggcgatcaacaaaaaataccgccatgcagacggaaccgaaatgaccatttcccgtgtctgctgggacatcggggggatcgatggcgaaatcgtttatcagaggtcaaaaaaacacggtgttttccgggtgctgccggtaaaaggcgcatctgtctatggcaagccggtgatcaccatgccaaaaacccgcaatcagcggggcgtgtatctgtgtgaagtggggacggacaccgcaaaagaaattctctatgcccgtatgaaagccgatcccacgcctgtggatgaagccacgtcgtatgctatccgttttcctgatgatccggagattttttcgcagacagaggcgcagcaactggtcgcggaagagcttgtggagaagtgggaaaaaggaaagatgcgtctgctgtgggataacaaaaagcggcgtaacgaagcgctggactgcctggtgtatgcctacgcggcattacgtgtgtccgtgcaacgctggcagcttgatctggctgtactggcaaaatcccgggaagaagagacgacccggccaacccttaaagaactggcagcgaagctgtccggaggagtgaatggttacagtcgctgaactgcaggcgctgcgtcaggcgcgccttgatttattaaccggtaaacgggtggtgtctgtccagaaagatggtcgcagaattgaatatacggcggcttctctggatgagcttaaccgggcgatcaatgatgcggagtcggtactggggacaacccggtgtcgccgtcgtccgctgggagtgaggttatgaaacgaacgcctgtcctgattgatgtgaacggcgttccgcttcgtgagagtctcagctacaacgggggcggtgcaggatttggcgggcaaatggctgagtggttgccaccggcgcagagtgccgatgcggccctgctgcccgcgttgcgtctggggaatgcccgggcagatgatctggtgcgcaataacggaatagcggctaatgcggtggctctgcataaggatcacattgtcgggcatatgtttctgatcagctaccgtccgaactggcgctggctggggatgcgggagaccgcagcaaaaagctttgtcgatgaggtggaggcggcctggtcggaatacgccgaagggatgtttggcgagatcgacgtggaaggaaaacgcacgttcacggaatttatccgtgaaggtgtgggcgttcatgcgtttaacggcgaaatctttgtgcagccggtctgggatacggaaaccacgcagttattccgtacgcgttttaaagccgtgagtccgaaacgggtggacacgcctggacacggtatggggaaccgttttctgcgggccggtgtggaggtcgatcgatatggccgtgccgtcgcgtaccatatctgtgaggatgattttccgttctctggtagtggacgatgggaacggatcccgcgtgaacttcccaccgggcgtccggccatgctgcatattttcgagccggtggaggacgggcagacccgtggggctaatcagttttacagcgtcatggaacggctgaagatgctcgattccctgcaggcaacacagcttcagtcggccatagtgaaggcgatgtatgcagcgacgattgaaagtgaacttgataccgaaaaggcctttgaatatatcgccggcgcgccacaggagcagaaggataatccgcttattaatattctggagaagttttccagctggtatgacacgaataacgtgacactgggcggtgtcaaaattccgcaccttttccctggtgatgatctgaaactacagactgcgcaggattcagacaatggattttctgcgcttgaacaggcgctgctgcggtatatcgccgccggtcttggcgtttcctacgaacagttgtcccgtgattactcgaaggtcagttactcaagtgcccgcgcctccgccaatgagtcgtggcgctattttatggggcggcgaaaatttattgcggcccggctggccacgcagatgttttcctgctggctggaagaggcacttcttcgggggattattcgtccgccacgggcacgttttgatttttatcaggcgcgatcagcctggtcacgggcagagtggattggtgccggaagaatggccattgacgggctcaaggaagtccaggaatcagtgatgcgcattgaggccggactgagcacgtatgagaaagggctggcgctgatgggcgaggattatcaggacattttccgccagcaggtcagggaatctgctgagcggcaaaaagccggactctcacgtccggtgtggatagagcaggcgtatcagcagcagatagcggagagtcgtaggccggaagaggagacaacaccacgtgagacgtaatctttcacacattattgccgcagcattcaatgaaccgctgcttctggagcccgcctatgcgcgggttttcttttgcgcgctcgggcgcgagatgggggcagcaagtctttcggtaccacaacagcaggtacagtttgatgctcccggaatgctggctgaaacggacgagtacatggccggaggtaaacgaccggcccgtgtttaccgggtggtgaacggtattgctgtactgccggtgaccggcacgctggtgcaccggctgggtggtatgcggccattttccggaatgacaggctatgacggcattgtcgcctgtcttcagcaggcaatggcggatagccaggtgcggggcgtactgctggacattgacagtccgggcgggcaggccgccggcgcgtttgactgcgctgacatgatttaccgcctccgtcagcagaagccggtctgggcactgtgcaatgacacggcctgttctgcagccatgctgctggcgtcggcctgctcccgacggctggttacccagacatcccgtatcggctccattggcgtgatgatgagccatgtcagctatgccggtcatctggcgcaggccggtgtggatatcacgctgatttactcaggggcgcacaaggtggatggcaatcagtttgaagcgttgccggcagaggttcgccaggacatgcagcagcggattgatgcggcgcgccggatgtttgccgaaaaagtggcgatgtataccggtctgtctgttgatgccgtcacgggaacagaggctgccgtttttgaaggtcagtccggcattgaggccgggctggcggatgaattaatcaatgcgtcggatgccatcagtgtgatggccacggcgctgaacagtaatgtcagaggaggcactatgccgcaattaactgcaacggaagccgccgcgcaggagaaccagcgagtgatggggatcctgacatgccaggaagcgaaaggacgtgaacagcttgccacgatgctggcaggacaacagggcatgagcgttgaacaggcccgggcgattctggccgcggcggcaccgcagcagccggtggcatccacgcagagtgaagccgatcgcattatggtgtgtgaagaagcgaacggtcgtgaacaactggcggcaacgctggcggcgatgccggagatgacggtggaaaaagcccgcccgatcctggctgcttcaccgcaggcggatgccggaccatcactccgtgatcagatcatggcactggatgaggcaaaaggggctgaggcgcaggctgaacagctggctgcctgcccgggaatgacagtggagagcgcccgggctgtgctggctgcgggatcaggtaaggcagaaccggtctctgcatccacaaccgccatgtttgaacatttcatggcgaaccattcaccggcagcggtacagggtggcgtggcacagacgtcagcagacggtgatgcggacgtgaaaatgctcatggccatgccatgaagtcagtgctgaccatcaataggaggtttttacaatatggtgacgaaaaccatcactgaacagcgtgcggaagtacgtatttttgccggtaatgatccggctcataccgccacaggcagcagcgggatttcctcgccaacaccggcactgacgcccctgatgctggatgaagccaccgggaaactggtggtctgggacggacagaaagccggtagtgcggttggcatactggtactgccgcttgaaggcacagagacggcgctgacgtattacaagtcgggaacctttgcgacggaggcaatccactggcctgaaagtgtggatgaacacaaaaaggccaacgcctttgctggcagtgccctgagtcacgcggcgctgccgtaacacgttatcaggccaccgcggtggcctgactgatttctgaatgaaaggaactgatttatgggattgtttacgacccgccagttactcggttataccgaacaaaaagttaaatttcgtgcgctgtttctggagctgtttttccgccgtacggtgaatttccataccgaagaggtgatgctggacaaaattaccggaaaaacgccggtggcggcctatgtctccccggttgttgaaggaaaagtgctgcgtcatcgtggtggtgaaacccgcgtgttgcgtccgggctacgtcaagccgaaacacgaatttaattaccagcaggcggttgagcgccttcctggtgaagatccatctcaactgaatgatccggcttaccgccgtctgcgtatcattaccgataacctcaaacaggaagagcacgcgattgtccaggtggaagaaatgcaggcggtaaatgctgtgttgtatggcaaatacaccatggaaggagaccagttcgagaaaattgaggtcgattttggcaggtcgacgaagaataacatcactcagggtagtggtaaggagtggtcaaaacaggatcgtgacacgttcgatcctacacatgatattgacctctactgcgacctggccagcggtcttgtgaatattgccattatggacggtaccgtctggcgtcttctgaatggttttaagctgttccgcgaaaaactggatacccgtcgcggttcaaattctcaactcgaaacggcagtgaaagatctgggcgcagtggtgtccttcaaggggtattacggcgatctggccattgtggtggcgaaaacgtcttatatagcagaagacggtatcgaaaaacgttatcttccagatggcatgctggttctggggaatactgctgcagatgggatccgttgttacggtgccattcaggatgctcaggcgttgtccgaaggtgtggtggcctcttcccgttatccgaaacactggctgacggtaggggatcccgcccgtgaatttaccatgacgcagtccgcgccgctgatggtgttgccggacccggatgagtttgtggtggtacaggtgaaataatccgtgagcgggggcgaaatgcccccgtgtcttttttcacagggggctgatatggcaacgaaagagcaaaatctgaaacggcttgatgaactggccctgattctggggcgtgagccggatatatccgggagtgccgcagagatagcgcagcgggtggcagaatgggaagaggaaatgcagtcatccggcgatgatgtacaggttatgaatatggatatccgggagcgggaaaccgcggctcatgatgttcgtgaggaaacatccggcgcgttaacgcgcatcagagttctgacctgcctccatctctgtggcgttgatggtgaaacgggggaatccgttgagcttgcggatgttggtcgggtgattctgattatgtcctcagatgcaaaaacacacgttgatggtggaatggctgtttatgcgtgattttcagaatgcctttgatgccgcccttgccggggtggacagtacgattgttgaagtgatgggcatcagtgcgcagttcacctccggtgcacagcgtggcggcgaggttcatggcgtttttgacgatccggagtcgctgggttttgccagtagtgggatcc

SEQ ID NO: 43 - protein, 49 aa, Artificial Sequence
  Description of Artificial Sequence; note = synethetic construct
QCESGPCCRNCKFLKEGTICKRARGDDMDDYCNGKTCDCPRNPHKGPAT

SEQ ID NO: 44 - protein, 341 aa, Artificial Sequence
  Description of Artificial Sequence; note = synethetic construct
MSMYTTAQLLAANEQKFKFDPLFLRLFFRESYPFTTEKVYLSQIPGLVNMALYVSPIVSGEVIRSRGGSTSEFTPGYVKPKHEVNPQMTLRRLPDEDPQNLADPAYRRRRIIMQNMRDEELAIAQVEEMQAVSAVLKGKYTMTGEAFDPVEVDMGRSEENNITQSGGTEWSKRDKSTYDPTDDIEAYALNASGVVNIIVFDPKGWALFRSFKAVKEKLDTRRGSNSELETAVKDLGKAVSYKGMYGDVAIVVYSGQYVENGVKKNFLPDNTMVLGNTQARGLRTYGCIQDADAQREGINASARYPKNWVTTGDPAREFTMIQSAPLMLLADPDEFVSVQLA

SEQ ID NO: 45 - protein, 341 aa, Artificial Sequence
  Description of Artificial Sequence; note = synethetic construct
MSMYTTAQLLAANEQKFKFDPLFLRLFFRESYPFTTEKVYLSQIPGLVNMALYVSPIVSGEVIRSRGGSTSEFTPGYVKPKHEVNPQMTLRRLPDEDPQNLADPAYRRRRIIMQNMRDEELAIAQVEEMQAVSAVLKGKYTMTGEAFDPVEVDMGRSEENNITQSGGTKWSKRDKSTYDPTDDIEAYALNASGVVNIIVFDPKGWALFRSFKAVKEKLDTRRGSNSELETAVKDLGKAVSYKGMYGDVAIVVYSGQYVENGVKKNFLPDNTMVLGNTQARGLRTYGCIQDADAQREGINASARYPKNWVTTGDPAREFTMIQSAPLMLLADPDEFVSVQLA

SEQ ID NO: 46 - protein, 5 aa, Artificial Sequence
  Description of Artificial Sequence; note = synethetic construct
CNGRC

SEQ ID NO: 47 - protein, 13 aa, Artificial Sequence
  Description of Artificial Sequence; note = synethetic construct
CNGRCVSGCAGRC

SEQ ID NO: 48 - protein, 9 aa, Artificial Sequence
  Description of Artificial Sequence; note = synethetic construct
CVLNGRMEC

SEQ ID NO: 49 - protein, 6 aa, Artificial Sequence
  Description of Artificial Sequence; note = synethetic construct
NGRAHA

SEQ ID NO: 50 - protein, 6 aa, Artificial Sequence
  Description of Artificial Sequence; note = synethetic construct
SAKTTP

SEQ ID NO: 51 - protein, 6 aa, Artificial Sequence
  Description of Artificial Sequence; note = synethetic construct
RADAAP

SEQ ID NO: 52 - DNA, 333 bp, Artificial Sequence
  Description of Artificial Sequence; note = synethetic construct
atgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaa

SEQ ID NO: 53 - DNA, 741 bp, Artificial Sequence
  Description of Artificial Sequence; note = synethetic construct
atgcctgtaccaaatcctacaatgccggtgaaaggtgccgggaccaccctgtgggtttataaggggagcggtgacccttacgcgaatccgctttcagacgttgactggtcgcgtctggcaaaagttaaagacctgacgcccggcgaactgaccgctgagtcctatgacgacagctatctcgatgatgaagatgcagactggactgcgaccgggcaggggcagaaatctgccggagataccagcttcacgctggcgtggatgcccggagagcaggggcagcaggcgctgctggcgtggtttaatgaaggcgatacccgtgcctataaaatccgcttcccgaacggcacggtcgatgtgttccgtggctgggtcagcagtatcggtaaggcggtgacggcgaaggaagtgatcacccgcacggtgaaagtcaccaatgtgggacgtccgtcgatggcagaagatcgcagcacggtaacagcggcaaccggcatgaccgtgacgcctgccagcacctcggtggtgaaagggcagagcaccacgctgaccgtggccttccagccggagggcgtaaccgacaagagctttcgtgcggtgtctgcggataaaacaaaagccaccgtgtcggtcagtggtatgaccatcaccgtgaacggcgttgctgcaggcaaggtcaacattccggttgtatccggtaatggtgagtttgctgcggttgcagaaattaccgtcaccgccagttaa

SEQ ID NO: 54 - protein, 15 aa, Artificial Sequence
  Description of Artificial Sequence; note = synethetic construct
GGGGSGGGGSGGGGS

SEQ ID NO: 55 - protein, 7 aa, Artificial Sequence
  Description of Artificial Sequence; note = synethetic construct
GSGSGSX

SEQ ID NO: 56 - protein, 10 aa, Artificial Sequence
  Description of Artificial Sequence; note = synethetic construct
RGPGRAFVTI

SEQ ID NO: 57 - DNA, 13 bp, Artificial Sequence
  Description of Artificial Sequence; note = synethetic construct
gsgggsgggsggt

SEQ ID NO: 58 - DNA, 20 bp, Artificial Sequence
  Description of Artificial Sequence; note = synethetic construct
aagctttcatgaccagcaag

SEQ ID NO: 59 - DNA, 32 bp, Artificial Sequence
  Description of Artificial Sequence; note = synethetic construct
atctaagcttctatacaatactgattgcggtg

SEQ ID NO: 60 - DNA, 30 bp, Artificial Sequence
  Description of Artificial Sequence; note = synethetic construct
atcggtacccaggtttctgatgttccgcgt

SEQ ID NO: 61 - DNA, 30 bp, Artificial Sequence
  Description of Artificial Sequence; note = synethetic construct
ggccaagcttctaggtacggtagttaatcg

SEQ ID NO: 62 - DNA, 32 bp, Artificial Sequence
  Description of Artificial Sequence; note = synethetic construct
dagctccatgggaagcacacggtcacactgct

SEQ ID NO: 63 - DNA, 32 bp, Artificial Sequence
  Description of Artificial Sequence; note = synethetic construct
agctgcatgcaagttagctcactcattaggga

SEQ ID NO: 64 - DNA, 32 bp, Artificial Sequence
  Description of Artificial Sequence; note = synethetic construct
ggtgcccatatggcgagcaaagaaacctttac

SEQ ID NO: 65 - DNA, 33 bp, Artificial Sequence
  Description of Artificial Sequence; note = synethetic construct
ccgacgggatcctcattaaacgatgctgattgc

SEQ ID NO: 66 - protein, 10 aa, Artificial Sequence
  Description of Artificial Sequence; note = synethetic construct
RGPGRAFVTI